SEQUENCE LISTING (184 sequences)

SEQ ID NO: 1 - RNA, 27 nt, synthetic construct
  source 1..27: mol_type = other RNA
ggctttctggctccttaccggaaagcc

SEQ ID NO: 2 - RNA, 27 nt, synthetic construct
  source 1..27: mol_type = other RNA
ggctttctgggaggttaccggaaagcc

SEQ ID NO: 3 - RNA, 28 nt, synthetic construct
  source 1..28: mol_type = other RNA
ggctttctggcctccttaccggaaagcc

SEQ ID NO: 4 - RNA, 28 nt, synthetic construct
  source 1..28: mol_type = other RNA
ggctttctggggaggttaccggaaagcc

SEQ ID NO: 5 - RNA, 27 nt, synthetic construct
  source 1..27: mol_type = other RNA
ggctttctggctggctaccggaaagcc

SEQ ID NO: 6 - RNA, 25 nt, synthetic construct
  source 1..25: mol_type = other RNA
ggctttctggcttccccggaaagcc

SEQ ID NO: 7 - RNA, 27 nt, synthetic construct
  source 1..27: mol_type = other RNA
ggctttctggcttcttcccggaaagcc

SEQ ID NO: 8 - RNA, 27 nt, synthetic construct
  source 1..27: mol_type = other RNA
ggctttctggcaacttaccggaaagcc

SEQ ID NO: 9 - RNA, 27 nt, synthetic construct
  source 1..27: mol_type = other RNA
ggctttctggttcggtaccggaaagcc

SEQ ID NO: 10 - RNA, 28 nt, synthetic construct
  source 1..28: mol_type = other RNA
ggctttctggaagccttaccggaaagcc

SEQ ID NO: 11 - RNA, 27 nt, synthetic construct
  source 1..27: mol_type = other RNA
ggctttctggcttcttaccggaaagcc

SEQ ID NO: 12 - RNA, 29 nt, synthetic construct
  source 1..29: mol_type = other RNA
ggctttctggcctccgccggaaagcccct

SEQ ID NO: 13 - RNA, 30 nt, synthetic construct
  source 1..30: mol_type = other RNA
ggctttctggctccttaccggaaagcccct

SEQ ID NO: 14 - RNA, 29 nt, synthetic construct
  source 1..29: mol_type = other RNA
ggttttctgacctccgtcggaaaacccct

SEQ ID NO: 15 - RNA, 33 nt, synthetic construct
  source 1..33: mol_type = other RNA
ggttttctgacctccttcggtcggaaaacccct

SEQ ID NO: 16 - RNA, 26 nt, synthetic construct
  source 1..26: mol_type = other RNA
ggttttctgacctccgtcggaaaacc

SEQ ID NO: 17 - RNA, 30 nt, synthetic construct
  source 1..30: mol_type = other RNA
ggttttctgacactccgtcggaaaacccct

SEQ ID NO: 18 - RNA, 29 nt, synthetic construct
  source 1..29: mol_type = other RNA
ggttttctgatctccatcggaaaacccct

SEQ ID NO: 19 - RNA, 29 nt, synthetic construct
  source 1..29: mol_type = other RNA
ggttttccgacctccgtcggaaaacccct

SEQ ID NO: 20 - RNA, 30 nt, synthetic construct
  source 1..30: mol_type = other RNA
ggctttctggcactccaccggaaagcccct

SEQ ID NO: 21 - RNA, 30 nt, synthetic construct
  source 1..30: mol_type = other RNA
ggctttctggcactccgccggaaagcccct

SEQ ID NO: 22 - RNA, 29 nt, synthetic construct
  source 1..29: mol_type = other RNA
ggctttctggcctccaccggaaagcccct

SEQ ID NO: 23 - RNA, 29 nt, synthetic construct
  source 1..29: mol_type = other RNA
ggttttccggtctccaccggaaagccccc

SEQ ID NO: 24 - RNA, 29 nt, synthetic construct
  source 1..29: mol_type = other RNA
ggctttccggtctccaccggaaagcccct

SEQ ID NO: 25 - RNA, 29 nt, synthetic construct
  source 1..29: mol_type = other RNA
ggctttccggcctccgccggaaagcccct

SEQ ID NO: 26 - RNA, 29 nt, synthetic construct
  source 1..29: mol_type = other RNA
ggttttccggtctccaccggaaaaccctt

SEQ ID NO: 27 - RNA, 29 nt, synthetic construct
  source 1..29: mol_type = other RNA
ggttttccgtgctcccacggaaaaccctt

SEQ ID NO: 28 - RNA, 29 nt, synthetic construct
  source 1..29: mol_type = other RNA
ggttttccggcctccgccggaaaaccctt

SEQ ID NO: 29 - RNA, 30 nt, synthetic construct
  source 1..30: mol_type = other RNA
ggttttccgtgactcccacggaaaaccctt

SEQ ID NO: 30 - RNA, 30 nt, synthetic construct
  source 1..30: mol_type = other RNA
ggttttccggcactccgccggaaaaccctt

SEQ ID NO: 31 - RNA, 29 nt, synthetic construct
  source 1..29: mol_type = other RNA
ggtcttccggtctcctccggaaggccccc

SEQ ID NO: 32 - RNA, 27 nt, synthetic construct
  source 1..27: mol_type = other RNA
ggtcttccggctccccggaaggccccc

SEQ ID NO: 33 - RNA, 29 nt, synthetic construct
  source 1..29: mol_type = other RNA
ggctttccgtgctcccacggaaagcccct

SEQ ID NO: 34 - RNA, 30 nt, synthetic construct
  source 1..30: mol_type = other RNA
ggctttccgtgactcccacggaaagcccct

SEQ ID NO: 35 - RNA, 30 nt, synthetic construct
  source 1..30: mol_type = other RNA
ggctttccggcactccgccggaaagcccct

SEQ ID NO: 36 - RNA, 29 nt, synthetic construct
  source 1..29: mol_type = other RNA
ggttttctgacttcggtcggaaaacccct

SEQ ID NO: 37 - RNA, 26 nt, synthetic construct
  source 1..26: mol_type = other RNA
ggttttctgacttcggtcggaaaacc

SEQ ID NO: 38 - RNA, 27 nt, synthetic construct
  source 1..27: mol_type = other RNA
ggctttctggctttttaccggaaagcc

SEQ ID NO: 39 - RNA, 30 nt, synthetic construct
  source 1..30: mol_type = other RNA
ggctttctggctttttaccggaaagcccct

SEQ ID NO: 40 - RNA, 29 nt, synthetic construct
  source 1..29: mol_type = other RNA
ggctttccggcctccgccggaaagcccct

SEQ ID NO: 41 - RNA, 26 nt, synthetic construct
  source 1..26: mol_type = other RNA
ggctttccggcctccgccggaaagcc

SEQ ID NO: 42 - DNA, 30 bp, synthetic construct
  source 1..30: mol_type = other DNA
caaactacagagccaagtgctatccacaga

SEQ ID NO: 43 - DNA, 30 bp, synthetic construct
  source 1..30: mol_type = other DNA
gagctttctgggttgccatctcaagcagac

SEQ ID NO: 44 - DNA, 30 bp, synthetic construct
  source 1..30: mol_type = other DNA
tacaaggccatcagctcatactcacaattg

SEQ ID NO: 45 - DNA, 100 bp, synthetic construct
  source 1..100: mol_type = other DNA
caaactacagagccaagtgctatccacagagagctttctgggttgccatctcaagcagactacaaggccatcagctcatactcacaattgactttgagag

SEQ ID NO: 46 - DNA, 100 bp, synthetic construct
  source 1..100: mol_type = other DNA
ttgaccacatacgtgctctttcaaagttctgtgtttgaagttatgttagtaacaactgatgcccatcctgcaatgacaaatccaattctcagtgcagctc

SEQ ID NO: 47 - DNA, 500 bp, synthetic construct
  source 1..500: mol_type = other DNA
caaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgt

SEQ ID NO: 48 - DNA, 500 bp, synthetic construct
  source 1..500: mol_type = other DNA
ttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaac

SEQ ID NO: 49 - RNA, 13 nt, synthetic construct
  source 1..13: mol_type = other RNA
aatttttggagca

SEQ ID NO: 50-57 - skipped (8 entries)

SEQ ID NO: 58 - DNA, 288 bp, Homo sapiens
  source 1..288: mol_type = genomic DNA
tactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 59 - DNA, 392 bp, Homo sapiens
  source 1..392: mol_type = genomic DNA
aaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttc

SEQ ID NO: 60 - DNA, 247 bp, Homo sapiens
  source 1..247: mol_type = genomic DNA
ccacgccctctgtgaaagggcggggcatgcaaattcgaaatgaaagcccgggaacgccggaagaagcacgggtgtaagatttcccttttcaaaggcagagaataagaaatcagcccgagagtgtaagggcgtcaatagcgctgtggacgagacagagggaatggggcaaggagcgaggctggggctctcaccgcgacttgaatgtggatgagagtgggacggtgacggcgggcgcgaaggcgagcgc

SEQ ID NO: 61 - DNA, 250 bp, Homo sapiens
  source 1..250: mol_type = genomic DNA
ttcgcagtctctgaattaagtctattagcatgttcctcccatagtgctttgcttcatatcaacaaaaacctagctaagtgaaatcagcaacgatatgcagaaaccacctacgcaggtcacaaacatctttctatgattgtataattttcaagcaagcaataagtgaagatttttccataggccctaaactcacctttgcgaaataggaagctggtttattgggagtgatgagcagggggcgtaacaaatt

SEQ ID NO: 62 - DNA, 300 bp, Homo sapiens
  source 1..300: mol_type = genomic DNA
gcagcaaggcctccacttcaccccctaaaggttgccccaagagcaccgtgtgactgctaaggtatttccggagtctaaagacgattattcaggtctcatttgcatacccataatacactgcaaacagtatttttttcggaaaaacatttatatattgcttgacatttttaagtatgagaattttgcatgcagaatttttttgtataaactttctcaggtagtaacccttgggattagtagacaccatcagtgtactaggaattgcagttacccgaaaattgagttacagaagtaactggt

SEQ ID NO: 63 - DNA, 296 bp, Homo sapiens
  source 1..296: mol_type = genomic DNA
gtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 64 - DNA, 243 bp, Homo sapiens
  source 1..243: mol_type = genomic DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 65 - DNA, 106 bp, Homo sapiens
  source 1..106: mol_type = genomic DNA
caggactagtcttttaggtcaaaaagaagaagctttgtaaccgttggtttccgtagtgtagtggttatcacgttcgcctaacacgcgaaaggtccccggttcgaag

SEQ ID NO: 66 - DNA, 439 bp, Mus musculus
  source 1..439: mol_type = genomic DNA
ttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactc

SEQ ID NO: 67 - DNA, 258 bp, Mus musculus
  source 1..258: mol_type = genomic DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaa

SEQ ID NO: 68 - DNA, 440 bp, Mus musculus
  source 1..440: mol_type = genomic DNA
tgccaacagctttgaaatcctctgtgctcttgtgccaatcccaccctcagaacagggagtctgtcggttatgttttcgtcctttacagttcttcgccgtctaatccattggaaacctcctttatttaaaggaaaccgtgtccaaattcagtgtacagaggtgcaaacctccgccccgctccttgtaggaagcccacgggaactgggccaatcggacggcgcttactcggctcatttacatacccataacacaccgcggctaatgcaaatattttcgtgaaaagatcttccattactcgaagttttaaaactaaaaacaaaatattatttcaaaagatcatgaagaaatttctgtcggtaacagcagtttcaattgataaatcaccatcacttatctaggaggttctgttactctagaagtgaattaagcaggacagctgt

SEQ ID NO: 69 - DNA, 231 bp, Mus musculus
  source 1..231: mol_type = genomic DNA
gggcggggcatgcaaataactgctctgtggaactctgggagcaaaaacaaaaaactgcaaccaaaacttctcggcctccttgaatcttacaggcttttcgtggcgtaaaggtggtgtactcaatgaagaggagagtctgtgttggctgcatgtttgagtcggttggttggtgactgtgaattaaaggtgtggtcggtgttgagtgtatggggcgtgtgggcgtagttcggt

SEQ ID NO: 70 - DNA, 311 bp, Mus musculus
  source 1..311: mol_type = genomic DNA
cgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttt

SEQ ID NO: 71 - DNA, 97 bp, Homo sapiens
  source 1..97: mol_type = genomic DNA
catgcaaattacgcgctgtgctttgtgggaaatcaccctaaacgtaaaatttattcctctttcgagccttatagtggcggccggtctacaccctaaa

SEQ ID NO: 72 - DNA, 94 bp, Homo sapiens
  source 1..94: mol_type = genomic DNA
cctcttatgatgtttgttgccaatgatagattgttttcactgtgcaaaaattatgggtagttttggtggtcttgatgcagttgtaagcttggag

SEQ ID NO: 73 - DNA, 50 bp, Homo sapiens
  source 1..50: mol_type = genomic DNA
actttctggagtttcaaaaacagactgtacgccaagggtcatatcttttt

SEQ ID NO: 74 - DNA, 50 bp, Homo sapiens
  source 1..50: mol_type = genomic DNA
ccggggatacaacgtgtttcctaaaagtagagggaggtaagagacggtag

SEQ ID NO: 75 - DNA, 50 bp, Homo sapiens
  source 1..50: mol_type = genomic DNA
ctgaattttcttgcagttgaacaacagaggctttttttgtgtgtgtgggg

SEQ ID NO: 76 - DNA, 50 bp, Homo sapiens
  source 1..50: mol_type = genomic DNA
atatgtggtaatccaacaatagaaattatttttaagtttgtgtgttcctt

SEQ ID NO: 77-79 - skipped (3 entries)

SEQ ID NO: 80 - DNA, 206 bp, Mus musculus
  source 1..206: mol_type = genomic DNA
gtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagca

SEQ ID NO: 81 - DNA, 107 bp, Mus musculus
  source 1..107: mol_type = genomic DNA
cccaatttcactggtctacaatgaaagcaaaacagttctcttccccgctccccggtgtgtgagaggggctttgatccttctctggtttcctaggaaacgcgtatgtg

SEQ ID NO: 82 - DNA, 110 bp, Mus musculus
  source 1..110: mol_type = genomic DNA
cccaatttcactggtctacaatgaaagcaaaacagttctcttccccgctccccggtgtgtgagaggggctttgatccttctctggtttcctaggaaacgcgtatgtgtac

SEQ ID NO: 83 - DNA, 91 bp, Homo sapiens
  source 1..91: mol_type = genomic DNA
cttatgatgtttgttgccaatgatagattgttttcactgtgcaaaaattatgggtagttttggtggtcttgatgcagttgtaagcttggag

SEQ ID NO: 84 - DNA, 200 bp, Mus musculus
  source 1..200: mol_type = genomic DNA
ccacattttgtgtttaaaaatagaaatatttaagtgagatcagtttaaatctgctttatctagggtgtctaactgcttgcatctttttaacttttccttatttgtgagaaggtctgtaaaacttattatatgttagtacactgtagctgtcttcagacactccagaagagggattcagatcaccttacagatggttttga

SEQ ID NO: 85 - DNA, 78 bp, Mus musculus
  source 1..78: mol_type = genomic DNA
ccctctggggagtaaagttggttttaaagtcagagcatggtgattgtagggcagtccaacttttttaaatatgctgtg

SEQ ID NO: 86-87 - skipped (2 entries)

SEQ ID NO: 88 - RNA, 55 nt, synthetic construct
  source 1..55: mol_type = other RNA
ggtgtcgagaagaggagaacaatatgctaaatgttgttctcgtctcctcgacacc

SEQ ID NO: 89 - DNA, 110 bp, synthetic construct
  source 1..110: mol_type = other DNA
ctgcgcgctcgctcgctcactgaggccgcccgggcaaagcccgggcgtcgggcgacctttggtcgcccggcctcagtgagcgagcgagcgcgcagagagggagtggggtt

SEQ ID NO: 90 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 91 - DNA, 256 bp, Mus musculus
  source 1..256: mol_type = genomic DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 92 - RNA, 13 nt, synthetic construct
  source 1..13: mol_type = other RNA
aatttttggagta

SEQ ID NO: 93 - DNA, 20 bp, synthetic construct
  source 1..20: mol_type = other DNA
cgcctacaacgtcaacatca

SEQ ID NO: 94 - DNA, 20 bp, synthetic construct
  source 1..20: mol_type = other DNA
tggtgcagatgaacttcagg

SEQ ID NO: 95 - DNA, 584 bp, synthetic construct
  source 1..584: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 96 - DNA, 97 bp, synthetic construct
  source 1..97: mol_type = other DNA
ctctggctaactagagaacccactgcttactggcttatcgaaattaatacgactcactatagggagacccaagctggctagcgtttaaacgggccct

SEQ ID NO: 97 - DNA, 708 bp, synthetic construct
  source 1..708: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 98 - DNA, 81 bp, synthetic construct
  source 1..81: mol_type = other DNA
ctgcagggcggaggaggcagcggcggaggaggcagcggcggaggaggcagcgcctgctcgcgatgctagagggctctgcca

SEQ ID NO: 99 - DNA, 717 bp, synthetic construct
  source 1..717: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 100 - DNA, 72 bp, synthetic construct
  source 1..72: mol_type = other DNA
aattccaccacactggactagtggatccgagctcggtaccaagcttaagtttaaaccgctgatcagcctcga

SEQ ID NO: 101 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 102 - RNA, 165 nt, synthetic construct
  source 1..165: mol_type = other RNA
ggtgtcgagaagaggagaacaatatgctaaatgttgttctcgtctcctcgacacctgaacagctcctcgcccttgctcactggcagagccctccagcatcgcgagcaggcgctgcctcctccgccaatttttggagtaggctttctggctccttaccggaaagcc

SEQ ID NO: 103 - RNA, 251 nt, synthetic construct
  source 1..251: mol_type = other RNA
ggagtggtgtcgagaagaggagaacaatatgctaaatgttgttctcgtctcctcgacacctcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtacaaatttttggagtaggctttctggctccttaccggaaagcc

SEQ ID NO: 104 - RNA, 170 nt, synthetic construct
  source 1..170: mol_type = other RNA
ggagtggtgtcgagaagaggagaacaatatgctaaatgttgttctcgtctcctcgacaccaaaccgagggatcataggggactgaatccaccattcttctcccaatccctgcaactccttcttcccctgcaatttttggagtaggctttctggctccttaccggaaagcc

SEQ ID NO: 105 - RNA, 251 nt, synthetic construct
  source 1..251: mol_type = other RNA
ggagtggtgtcgagaagaggagaacaatatgctaaatgttgttctcgtctcctcgacaccactacagttgctccgatatttaggctacgtcaataggcactaacttattggcgctggtgaacggacttcctctcgagtaccagaagatgactacaaaactcctttccattgcgagtatcggagtctggctcagtttggccagggaggcactaatttttggagtaggctttctggctccttaccggaaagcc

SEQ ID NO: 106 - RNA, 165 nt, synthetic construct; identical to SEQ ID NO: 102
  source 1..165: mol_type = other RNA

SEQ ID NO: 107 - RNA, 246 nt, synthetic construct
  source 1..246: mol_type = other RNA
ggtgtcgagaagaggagaacaatatgctaaatgttgttctcgtctcctcgacacctcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtacaaatttttggagtaggctttctggctccttaccggaaagcc

SEQ ID NO: 108 - RNA, 165 nt, synthetic construct
  source 1..165: mol_type = other RNA
ggtgtcgagaagaggagaacaatatgctaaatgttgttctcgtctcctcgacaccaaaccgagggatcataggggactgaatccaccattcttctcccaatccctgcaactccttcttcccctgcaatttttggagtaggctttctggctccttaccggaaagcc

SEQ ID NO: 109 - RNA, 246 nt, synthetic construct
  source 1..246: mol_type = other RNA
ggtgtcgagaagaggagaacaatatgctaaatgttgttctcgtctcctcgacaccactacagttgctccgatatttaggctacgtcaataggcactaacttattggcgctggtgaacggacttcctctcgagtaccagaagatgactacaaaactcctttccattgcgagtatcggagtctggctcagtttggccagggaggcactaatttttggagtaggctttctggctccttaccggaaagcc

SEQ ID NO: 110 - RNA, 115 nt, synthetic construct
  source 1..115: mol_type = other RNA
ggagttgaacagctcctcgcccttgctcactggcagagccctccagcatcgcgagcaggcgctgcctcctccgccaatttttggagtaggctttctggctccttaccggaaagcc

SEQ ID NO: 111 - RNA, 196 nt, synthetic construct
  source 1..196: mol_type = other RNA
ggagttcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtacaaatttttggagtaggctttctggctccttaccggaaagcc

SEQ ID NO: 112 - RNA, 115 nt, synthetic construct
  source 1..115: mol_type = other RNA
ggagtaaaccgagggatcataggggactgaatccaccattcttctcccaatccctgcaactccttcttcccctgcaatttttggagtaggctttctggctccttaccggaaagcc

SEQ ID NO: 113 - RNA, 196 nt, synthetic construct
  source 1..196: mol_type = other RNA
ggagtactacagttgctccgatatttaggctacgtcaataggcactaacttattggcgctggtgaacggacttcctctcgagtaccagaagatgactacaaaactcctttccattgcgagtatcggagtctggctcagtttggccagggaggcactaatttttggagtaggctttctggctccttaccggaaagcc

SEQ ID NO: 114 - RNA, 170 nt, synthetic construct
  source 1..170: mol_type = other RNA
ggagtggtgtcgagaagaggagaacaatatgctaaatgttgttctcgtctcctcgacacctgaacagctcctcgcccttgctcactggcagagccctctagcatcgcgagcaggcgctgcctcctccgccaatttttggagtaggctttctggctccttaccggaaagcc

SEQ ID NO: 115 - RNA, 251 nt, synthetic construct
  source 1..251: mol_type = other RNA
ggagtggtgtcgagaagaggagaacaatatgctaaatgttgttctcgtctcctcgacacctcgccgtccagctcgaccaggatgggcaccaccccggtgaacagctcctcgcccttgctcactggcagagccctctagcatcgcgagcaggcgctgcctcctccgccgctgcctcctccgccgctgcctcctccgccctgcagcttgtacaaatttttggagtaggctttctggctccttaccggaaagcc

SEQ ID NO: 116 - RNA, 165 nt, synthetic construct
  source 1..165: mol_type = other RNA
ggtgtcgagaagaggagaacaatatgctaaatgttgttctcgtctcctcgacacctgaacagctcctcgcccttgctcactggcagagccctctagcatcgcgagcaggcgctgcctcctccgccaatttttggagtaggctttctggctccttaccggaaagcc

SEQ ID NO: 117 - RNA, 246 nt, synthetic construct
  source 1..246: mol_type = other RNA
ggtgtcgagaagaggagaacaatatgctaaatgttgttctcgtctcctcgacacctcgccgtccagctcgaccaggatgggcaccaccccggtgaacagctcctcgcccttgctcactggcagagccctctagcatcgcgagcaggcgctgcctcctccgccgctgcctcctccgccgctgcctcctccgccctgcagcttgtacaaatttttggagtaggctttctggctccttaccggaaagcc

SEQ ID NO: 118 - RNA, 115 nt, synthetic construct
  source 1..115: mol_type = other RNA
ggagttgaacagctcctcgcccttgctcactggcagagccctctagcatcgcgagcaggcgctgcctcctccgccaatttttggagtaggctttctggctccttaccggaaagcc

SEQ ID NO: 119 - RNA, 196 nt, synthetic construct
  source 1..196: mol_type = other RNA
ggagttcgccgtccagctcgaccaggatgggcaccaccccggtgaacagctcctcgcccttgctcactggcagagccctctagcatcgcgagcaggcgctgcctcctccgccgctgcctcctccgccgctgcctcctccgccctgcagcttgtacaaatttttggagtaggctttctggctccttaccggaaagcc

SEQ ID NO: 120 - RNA, 70 nt, synthetic construct
  source 1..70: mol_type = other RNA
tgaacagctcctcgcccttgctcactggcagagccctccagcatcgcgagcaggcgctgcctcctccgcc

SEQ ID NO: 121 - RNA, 151 nt, synthetic construct
  source 1..151: mol_type = other RNA
tcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtaca

SEQ ID NO: 122 - RNA, 70 nt, synthetic construct
  source 1..70: mol_type = other RNA
tgaacagctcctcgcccttgctcactggcagagccctctagcatcgcgagcaggcgctgcctcctccgcc

SEQ ID NO: 123 - RNA, 151 nt, synthetic construct
  source 1..151: mol_type = other RNA
tcgccgtccagctcgaccaggatgggcaccaccccggtgaacagctcctcgcccttgctcactggcagagccctctagcatcgcgagcaggcgctgcctcctccgccgctgcctcctccgccgctgcctcctccgccctgcagcttgtaca

SEQ ID NO: 124 - RNA, 70 nt, synthetic construct
  source 1..70: mol_type = other RNA
ccgtcgcccttcagcacgcacacggccttcgtcgccataactcgctaggccacgccgaggtcctggttcc

SEQ ID NO: 125 - RNA, 70 nt, synthetic construct
  source 1..70: mol_type = other RNA
ccgtcgcccttcagcacgcacacggccttcgtcgccacaactcgctaggccacgccgaggtcctggttcc

SEQ ID NO: 126 - RNA, 111 nt, synthetic construct
  source 1..111: mol_type = other RNA
atgatgccctgcactgggccgtcgcccttcagcacgcacacggccttcgtcgccataactcgctaggccacgccgaggtcctggttccgaggactgcaacggaaaccccag

SEQ ID NO: 127 - RNA, 111 nt, synthetic construct
  source 1..111: mol_type = other RNA
atgatgccctgcactgggccgtcgcccttcagcacgcacacggccttcgtcgccacaactcgctaggccacgccgaggtcctggttccgaggactgcaacggaaaccccag

SEQ ID NO: 128 - RNA, 151 nt, synthetic construct
  source 1..151: mol_type = other RNA
tccttctgctcgaaattgatgatgccctgcactgggccgtcgcccttcagcacgcacacggccttcgtcgccataactcgctaggccacgccgaggtcctggttccgaggactgcaacggaaaccccagacgctgcaggagactacgacgc

SEQ ID NO: 129 - RNA, 74 nt, synthetic construct
  source 1..74: mol_type = other RNA
gtcgccataactcgctaggcaaacgccgaggtcctggtaaagcaacggaaaccccaaaagcaggagactacgac

SEQ ID NO: 130 - RNA, 74 nt, synthetic construct
  source 1..74: mol_type = other RNA
gtcgccacaactcgctaggcaaacgccgaggtcctggtaaagcaacggaaaccccaaaagcaggagactacgac

SEQ ID NO: 131 - RNA, 74 nt, synthetic construct
  source 1..74: mol_type = other RNA
gccctgcactgggccaaaagcacgcacacggccaaagtcgccataactcgctaggcaaagcaacggaaacccca

SEQ ID NO: 132 - RNA, 74 nt, synthetic construct
  source 1..74: mol_type = other RNA
gccctgcactgggccaaaagcacgcacacggccaaagtcgccacaactcgctaggcaaagcaacggaaacccca

SEQ ID NO: 133 - RNA, 71 nt, synthetic construct
  source 1..71: mol_type = other RNA
gtcgcccttcagcacgcacacggccttcgtcgccataactcgctaggccacgccgaggtcctggttccgag

SEQ ID NO: 134 - RNA, 71 nt, synthetic construct
  source 1..71: mol_type = other RNA
gtcgcccttcagcacgcacacggccttcgtcgccacaactcgctaggccacgccgaggtcctggttccgag

SEQ ID NO: 135 - RNA, 94 nt, synthetic construct
  source 1..94: mol_type = other RNA
gccctgcactgggccaaaagcacgcacacggccaaagtcgccacaactcgctaggccacgccgaggtcctggttccaaagcaacggaaacccca

SEQ ID NO: 136 - RNA, 47 nt, synthetic construct
  source 1..47: mol_type = other RNA
cgtcgccacaactcgctaggccacgccgaggtcctggttccgaggac

SEQ ID NO: 137 - RNA, 71 nt, synthetic construct
  source 1..71: mol_type = other RNA
gcactgggccgtcgcccttcagcacgcacacggccttcgtcgccacaactcgctaggccacgccgaggtcc

SEQ ID NO: 138 - RNA, 71 nt, synthetic construct
  source 1..71: mol_type = other RNA
atgatgccctgcactgggccgtcgcccttcagcacgcacacggccttcgtcgccacaactcgctaggccac

SEQ ID NO: 139 - RNA, 71 nt, synthetic construct
  source 1..71: mol_type = other RNA
ctcgaaattgatgatgccctgcactgggccgtcgcccttcagcacgcacacggccttcgtcgccacaactc

SEQ ID NO: 140 - RNA, 111 nt, synthetic construct
  source 1..111: mol_type = other RNA
tttccttctgctcgaaattgatgatgccctgcactgggccgtcgcccttcagcacgcacacggccttcgtcgccacaactcgctaggccacgccgaggtcctggttccgag

SEQ ID NO: 141 - RNA, 111 nt, synthetic construct
  source 1..111: mol_type = other RNA
ggtccattactttccttctgctcgaaattgatgatgccctgcactgggccgtcgcccttcagcacgcacacggccttcgtcgccacaactcgctaggccacgccgaggtcc

SEQ ID NO: 142 - RNA, 111 nt, synthetic construct
  source 1..111: mol_type = other RNA
caccttcactggtccattactttccttctgctcgaaattgatgatgccctgcactgggccgtcgcccttcagcacgcacacggccttcgtcgccacaactcgctaggccac

SEQ ID NO: 143 - RNA, 151 nt, synthetic construct
  source 1..151: mol_type = other RNA
tccattactttccttctgctcgaaattgatgatgccctgcactgggccgtcgcccttcagcacgcacacggccttcgtcgccacaactcgctaggccacgccgaggtcctggttccgaggactgcaacggaaaccccagacgctgcaggag

SEQ ID NO: 144 - RNA, 151 nt, synthetic construct
  source 1..151: mol_type = other RNA
ccttcactggtccattactttccttctgctcgaaattgatgatgccctgcactgggccgtcgcccttcagcacgcacacggccttcgtcgccacaactcgctaggccacgccgaggtcctggttccgaggactgcaacggaaaccccagac

SEQ ID NO: 145 - RNA, 151 nt, synthetic construct
  source 1..151: mol_type = other RNA
cttccccacaccttcactggtccattactttccttctgctcgaaattgatgatgccctgcactgggccgtcgcccttcagcacgcacacggccttcgtcgccacaactcgctaggccacgccgaggtcctggttccgaggactgcaacgga

SEQ ID NO: 146 - RNA, 151 nt, synthetic construct
  source 1..151: mol_type = other RNA
tcctcggaaccaggacctcggcgtggcctagcgagttgtggcgacgaaggccgtgtgcgtgctgaagggcgacggcccagtgcagggcatcatcaatttcgagcagaaggaaagtaatggaccagtgaaggtgtggggaagcattaaagga

SEQ ID NO: 147 - RNA, 151 nt, synthetic construct
  source 1..151: mol_type = other RNA
cttcagtcagtcctttaatgcttccccacaccttcactggtccattactttccttctgctcgaaattgatgatgccctgcactgggccgtcgcccttcagcacgcacacggccttcgtcgccacaactcgctaggccacgccgaggtcctg

SEQ ID NO: 148 - RNA, 81 nt, synthetic construct
  source 1..81: mol_type = other RNA
gccctgcactgggccgaaacagcacgcacacggccaaagtcgccacaactcgctaggcaaagcaacggaaaccccagacgc

SEQ ID NO: 149 - RNA, 101 nt, synthetic construct
  source 1..101: mol_type = other RNA
gccctgcactgggccgaaacagcacgcacacggccaaagtcgccacaactcgctaggccacgccgaggtcctggttccaaagcaacggaaaccccagacgc

SEQ ID NO: 150 - RNA, 107 nt, synthetic construct
  source 1..107: mol_type = other RNA
gccctgcactgggccgaaacagcacgcacacggccaaagtcgccacaactcgctaggccacgccgaggtcctggttccgaggacaaagcaacggaaaccccagacgc

SEQ ID NO: 151 - RNA, 70 nt, synthetic construct; identical to SEQ ID NO: 120
  source 1..70: mol_type = other RNA

SEQ ID NO: 152 - RNA, 151 nt, synthetic construct; identical to SEQ ID NO: 121
  source 1..151: mol_type = other RNA

SEQ ID NO: 153 - RNA, 105 nt, synthetic construct
  source 1..105: mol_type = other RNA
gcccaccgtgtggttgctgtccaggacggtcccggcctgcgacacttcggcccagagctgctcctcatccagcagcgccagcagccccatggccgtgagcaccgg

SEQ ID NO: 154 - RNA, 79 nt, synthetic construct
  source 1..79: mol_type = other RNA
ttcggcccagagctgctcctaaaggccgtgagcaccggaaacgtgcggcgggcgggaaagcgcgaaggggtgcgggtgg

SEQ ID NO: 155 - RNA, 105 nt, synthetic construct
  source 1..105: mol_type = other RNA
gctacgtcaataggcactaacttattggcgctggtgaacggacttcctctcgagtaccagaagatgactacaaaactcctttccattgcgagtatcggagtctgg

SEQ ID NO: 156 - RNA, 71 nt, synthetic construct
  source 1..71: mol_type = other RNA
tggcaattttaaatacaaggtgcacctttgttatttgcaaaccttgaaagaaataaacttatttttaaaaa

SEQ ID NO: 157 - RNA, 71 nt, synthetic construct
  source 1..71: mol_type = other RNA
tggcaattttaaatacaaggtgcacctttgttattcgtaaaccttgaaagaaataaacttatttttaaaaa

SEQ ID NO: 158 - RNA, 71 nt, synthetic construct
  source 1..71: mol_type = other RNA
tggcaattttaaatacaaggtgcacctttgttattcgcaaaccttgaaagaaataaacttatttttaaaaa

SEQ ID NO: 159 - RNA, 151 nt, synthetic construct
  source 1..151: mol_type = other RNA
ttaaacaaaaatgactgtgcatgcacgctctcatctataatggcaattttaaatacaaggtgcacctttgttatttgcaaaccttgaaagaaataaacttatttttaaaaaattatatcttggtctacagacgtaccaatacataatagaa

SEQ ID NO: 160 - RNA, 151 nt, synthetic construct
  source 1..151: mol_type = other RNA
ttaaacaaaaatgactgtgcatgcacgctctcatctataatggcaattttaaatacaaggtgcacctttgttattcgtaaaccttgaaagaaataaacttatttttaaaaaattatatcttggtctacagacgtaccaatacataatagaa

SEQ ID NO: 161 - RNA, 151 nt, synthetic construct
  source 1..151: mol_type = other RNA
ttaaacaaaaatgactgtgcatgcacgctctcatctataatggcaattttaaatacaaggtgcacctttgttattcgcaaaccttgaaagaaataaacttatttttaaaaaattatatcttggtctacagacgtaccaatacataatagaa

SEQ ID NO: 162 - RNA, 71 nt, synthetic construct
  source 1..71: mol_type = other RNA
tggatacactaattcttttaaacaagtgcccattaccacattttgcatttttgagtgcttctgagttgaaa

SEQ ID NO: 163 - RNA, 71 nt, synthetic construct
  source 1..71: mol_type = other RNA
tggatacactaattcttttaaacaagtgcccattatcacactttgcatttttgagtgcttctgagttgaaa

SEQ ID NO: 164 - RNA, 71 nt, synthetic construct
  source 1..71: mol_type = other RNA
tggatacactaattcttttaaacaagtgcccattatcacatcttgcatttttgagtgcttctgagttgaaa

SEQ ID NO: 165 - RNA, 71 nt, synthetic construct
  source 1..71: mol_type = other RNA
tggatacactaattcttttaaacaagtgcccattaccacaccttgcatttttgagtgcttctgagttgaaa

SEQ ID NO: 166 - RNA, 111 nt, synthetic construct
  source 1..111: mol_type = other RNA
taaccagctggagtgaaggctggatacactaattcttttaaacaagtgcccattaccacattttgcatttttgagtgcttctgagttgaaaattaaaaccatgaaatttga

SEQ ID NO: 167 - RNA, 111 nt, synthetic construct
  source 1..111: mol_type = other RNA
taaccagctggagtgaaggctggatacactaattcttttaaacaagtgcccattatcacactttgcatttttgagtgcttctgagttgaaaattaaaaccatgaaatttga

SEQ ID NO: 168 - RNA, 111 nt, synthetic construct
  source 1..111: mol_type = other RNA
taaccagctggagtgaaggctggatacactaattcttttaaacaagtgcccattatcacatcttgcatttttgagtgcttctgagttgaaaattaaaaccatgaaatttga

SEQ ID NO: 169 - RNA, 111 nt, synthetic construct
  source 1..111: mol_type = other RNA
taaccagctggagtgaaggctggatacactaattcttttaaacaagtgcccattaccacaccttgcatttttgagtgcttctgagttgaaaattaaaaccatgaaatttga

SEQ ID NO: 170 - RNA, 151 nt, synthetic construct
  source 1..151: mol_type = other RNA
gctgataagtgcaacatttttaaccagctggagtgaaggctggatacactaattcttttaaacaagtgcccattaccacattttgcatttttgagtgcttctgagttgaaaattaaaaccatgaaatttgaactgcgttttatttttcacc

SEQ ID NO: 171 - RNA, 151 nt, synthetic construct
  source 1..151: mol_type = other RNA
gctgataagtgcaacatttttaaccagctggagtgaaggctggatacactaattcttttaaacaagtgcccattatcacactttgcatttttgagtgcttctgagttgaaaattaaaaccatgaaatttgaactgcgttttatttttcacc

SEQ ID NO: 172 - RNA, 151 nt, synthetic construct
  source 1..151: mol_type = other RNA
gctgataagtgcaacatttttaaccagctggagtgaaggctggatacactaattcttttaaacaagtgcccattatcacatcttgcatttttgagtgcttctgagttgaaaattaaaaccatgaaatttgaactgcgttttatttttcacc

SEQ ID NO: 173 - RNA, 151 nt, synthetic construct
  source 1..151: mol_type = other RNA
gctgataagtgcaacatttttaaccagctggagtgaaggctggatacactaattcttttaaacaagtgcccattaccacaccttgcatttttgagtgcttctgagttgaaaattaaaaccatgaaatttgaactgcgttttatttttcacc

SEQ ID NO: 174 - RNA, 70 nt, synthetic construct
  source 1..70: mol_type = other RNA
aaaccgagggatcataggggactgaatccaccattcttctcccaatccctgcaactccttcttcccctgc

SEQ ID NO: 175 - RNA, 151 nt, synthetic construct; identical to SEQ ID NO: 123
  source 1..151: mol_type = other RNA

SEQ ID NO: 176 - RNA, 151 nt, synthetic construct; identical to SEQ ID NO: 123
  source 1..151: mol_type = other RNA

SEQ ID NO: 177 - DNA, 1630 bp, synthetic construct
  source 1..1630: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtgcccaccgtgtggttgctgtccaggacggtcccggcctgcgacacttcggcccagagctgctcctcatccagcagcgccagcagccccatggccgtgagcaccggaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtgcccaccgtgtggttgctgtccaggacggtcccggcctgcgacacttcggcccagagctgctcctcatccagcagcgccagcagccccatggccgtgagcaccggaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 178 - DNA, 1740 bp, synthetic construct
  source 1..1740: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtggtgtcgagaagaggagaacaatatgctaaatgttgttctcgtctcctcgacaccgcccaccgtgtggttgctgtccaggacggtcccggcctgcgacacttcggcccagagctgctcctcatccagcagcgccagcagccccatggccgtgagcaccggaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtggtgtcgagaagaggagaacaatatgctaaatgttgttctcgtctcctcgacaccgcccaccgtgtggttgctgtccaggacggtcccggcctgcgacacttcggcccagagctgctcctcatccagcagcgccagcagccccatggccgtgagcaccggaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 179 - DNA, 1688 bp, synthetic construct
  source 1..1688: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtggtgtcgagaagaggagaacaatatgctaaatgttgttctcgtctcctcgacaccttcggcccagagctgctcctaaaggccgtgagcaccggaaacgtgcggcgggcgggaaagcgcgaaggggtgcgggtggaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtggtgtcgagaagaggagaacaatatgctaaatgttgttctcgtctcctcgacaccttcggcccagagctgctcctaaaggccgtgagcaccggaaacgtgcggcgggcgggaaagcgcgaaggggtgcgggtggaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 180 - DNA, 1740 bp, synthetic construct
  source 1..1740: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtggtgtcgagaagaggagaacaatatgctaaatgttgttctcgtctcctcgacaccgctacgtcaataggcactaacttattggcgctggtgaacggacttcctctcgagtaccagaagatgactacaaaactcctttccattgcgagtatcggagtctggaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtggtgtcgagaagaggagaacaatatgctaaatgttgttctcgtctcctcgacaccgctacgtcaataggcactaacttattggcgctggtgaacggacttcctctcgagtaccagaagatgactacaaaactcctttccattgcgagtatcggagtctggaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 181 - DNA, 1124 bp, synthetic construct
  source 1..1124: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggtacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtgcccaccgtgtggttgctgtccaggacggtcccggcctgcgacacttcggcccagagctgctcctcatccagcagcgccagcagccccatggccgtgagcaccggaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 182 - DNA, 1179 bp, synthetic construct
  source 1..1179: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggtacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtggtgtcgagaagaggagaacaatatgctaaatgttgttctcgtctcctcgacaccgcccaccgtgtggttgctgtccaggacggtcccggcctgcgacacttcggcccagagctgctcctcatccagcagcgccagcagccccatggccgtgagcaccggaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 183 - DNA, 1152 bp, synthetic construct
  source 1..1152: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggtcgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtggtgtcgagaagaggagaacaatatgctaaatgttgttctcgtctcctcgacaccttcggcccagagctgctcctaaaggccgtgagcaccggaaacgtgcggcgggcgggaaagcgcgaaggggtgcgggtggaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 184 - DNA, 1179 bp, synthetic construct
  source 1..1179: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggtacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtggtgtcgagaagaggagaacaatatgctaaatgttgttctcgtctcctcgacaccgctacgtcaataggcactaacttattggcgctggtgaacggacttcctctcgagtaccagaagatgactacaaaactcctttccattgcgagtatcggagtctggaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

(19) United States
(12) Patent Application Publication
(10) Pub. No.: US 2026/0144893 A1
(43) Pub. Date: May 28, 2026

(54) COMPOSITIONS AND METHODS COMPRISING PROGRAMMABLE SNRNAS FOR RNA EDITING

(71) Applicant: Regeneron Pharmaceuticals, Inc., Tarrytown, NY (US)

(72) Inventors: Rea LARDELLI MARKMILLER, San Diego, CA (US); Ranjan BATRA, San Diego, CA (US); Rachel A. ADAMS, San Diego, CA (US); Alberto CARRENO, San Diego, CA (US)

(21) Appl. No.: 19/121,970

(22) PCT Filed: Oct. 18, 2023
(86) PCT No.: PCT/US2023/077197

§ 371 Date: Apr. 17, 2025

Related U.S. Application Data
(60) Provisional application No. 63/379,981, filed on Oct. 18, 2022.

(51) Int. Cl. A61K 48/00; A61K 38/47; A61P 3/00; A61P 21/00; C12N 9/78; C12N 15/11; C12N 15/113; C12N 15/86

(52) U.S. Cl. CPC A61K 48/005; A61K 38/47; A61P 3/00; A61P 21/00; C12N 9/78; C12N 15/111; C12N 15/113; C12N 15/86; C12Y 302/01076; C12N 2310/11; C12N 2310/531; C12N 2320/33; C12N 2750/14143; C12Y 305/04004

(57) ABSTRACT

SnRNA systems comprising RNA binding sequences for RNA editing are disclosed herein.

### RELATED APPLICATIONS

This application claims priority to, and the benefit of U.S. Provisional Application No. 63/379,981 filed Oct. 18, 2022. The contents of which are hereby incorporated by reference in its entirety.

### FIELD OF THE DISCLOSURE

The disclosure is directed to molecular biology, gene therapy, and compositions and methods for modifying expression and activity of RNA molecules.

### REFERENCE TO AN ELECTRONIC SEQUENCE LISTING

The contents of the electronic sequence listing (LOCN_022_001WO_SeqList_ST26.xml; Size: 182,228 bytes; and Date of Creation: Oct. 18, 2023) are herein incorporated by reference in its entirety.

### BACKGROUND

There are long-felt but unmet needs in the art for providing effective therapies for correcting dysfunctional messenger RNA.

Small nuclear RNA (snRNA) is one of the smallest types of RNA with an average size of about 150 nucleotides. snRNAs are functional non-coding RNAs. Eucaryotic genomes code for a variety of non-coding RNA such as snRNA, a class of highly abundant RNA, localized in the nucleus with important functions in intron splicing and RNA processing. snRNA, in the pre-mRNA splicing process, are capable of forming ribonucleoprotein particles (snRNPs) along with other proteins. These snRNPs and additional proteins form a large particulate complex (spliceosome) bound to the unspliced pre-mRNA transcripts. In addition to splicing. snRNAs function in nuclear maturation of nascent transcripts, gene expression regulation, as a splice donor in non-canonical systems, and in 3′ end processing of replication-dependent histone mRNAs. U7 snRNA can be programmed to bind and modulate mRNA, including editing of one or more bases, without exogenous protein expression but there still exists a need to develop a highly specific mRNA-targeting therapeutics that minimizes immunogenic risk. Furthermore, the small size of these programmed snRNAs creates an opportunity to develop single vector, highly specific (allele-specific), single target and multi-targeting gene therapy approaches.

Adenosine Deaminase That Acts on RNAs (ADAR) are a class of RNA-binding enzymes that convert adenosine (A) to inosine (I). The resultant inosine pairs with cytosine and is recognized as guanine by translational machinery. As a result, the use of ADARs to introduce A to I mutations in RNA molecules such as pre-mRNA or mRNA offers a means to edit these RNA molecules in a sequence-specific fashion. Such editing of pre-mRNA or mRNA molecules can be used for editing of premature termination codons to restore reading frame to an mRNA, editing splice regulatory sequences (bpA, 3′ss, ESEs) to produce knockdown of an mRNA of interest, and editing of translation start codons (ATG) to repress translation, amongst other things.

Accordingly, the disclosure provides compositions and methods comprising a new therapeutic RNA-targeting platform comprised of snRNAs comprising a base-pairing mismatch binding sequence that recruit ADARs to RNA sequences of interest to induce A to I editing events.

### SUMMARY

The disclosure provides an RNA-targeting nucleic acid molecule comprising a small nuclear RNA (snRNA) molecule, wherein the snRNA molecule comprises at least one targeting sequence, wherein the targeting sequence has at least one base-pairing mismatch, an Sm binding domain (SmBD), and an snRNA stem loop.

In some embodiments, the snRNA further comprises an Adenosine Deaminase That Acts on RNA (ADAR) recruiting domain. In some embodiments, the ADAR recruiting domain comprises a nucleotide sequence set forth in SEQ ID NO: 88.

In some embodiments, the snRNA further a 5′ interaction stability domain (5′ISD). In some embodiments, the snRNA stem loop is a native stem loop or an engineered stem loop (eSL).

In some embodiments, the snRNA is a U1 snRNA, U2 snRNA. U3 snRNA, U4 snRNA, U5 snRNA, U6 snRNA, or U7 snRNA. In some embodiments, the snRNA is a U7 snRNA.

In some embodiments, the targeting sequence binds an mRNA or pre-mRNA sequence.

In some embodiments, the targeting sequence binds a start codon, a stop codon, or a splicing regulatory sequence. In some embodiments, the splicing regulatory sequence is a branch point adenosine (bpA) sequence, 3′ acceptor splice site (3′ ss) sequence, or exonic splicing enhancer (ESE) sequence.

In some embodiments, the base-pairing mismatch is an adenosine (A)-cytosine (C) mismatch. In some embodiments, the adenosine is comprised within the mRNA or pre-mRNA sequence. In some embodiments, the cytosine is comprised within the snRNA targeting sequence.

In some embodiments, the targeting sequence is at least 50 nucleotides in length.

In some embodiments, the snRNA further comprises one or more additional targeting sequences. In some embodiments, the one or more additional targeting sequences have at least one base-pairing mismatch.

In some embodiments, the Sm binding domain (SmBD) is selected from the group consisting of a U1, U2, U4, and U5 SmBD.

In some embodiments, the interaction stability domain (5′ISD) comprises ggagt, cctct, ggaggt, cctcct, agccag, ggaag, gaagaag, gttg, ccgaa, taaggag, gaag, or ggctt.

In some embodiments, the snRNA stem loop comprises a nucleotide sequence set forth in any one of SEQ ID NOs: 1-41.

In some embodiments, the targeting sequence binds a SOD1 RNA sequence. In some embodiments, the SOD1 RNA targeting sequence comprises a nucleotide sequence set forth in any one of SEQ ID NOs: 124-150.

In some embodiments, the targeting sequence binds an IDUA RNA sequence. In some embodiments, the IDUA RNA targeting sequence comprises a nucleotide sequence set forth in any one of SEQ ID NOs: 153 or 154.

In some embodiments, the targeting sequence binds an MBNL1 RNA sequence. In some embodiments, the MBNL1 RNA targeting sequence comprises a nucleotide sequence set forth in any one of SEQ ID NOs: 156-173.

In some embodiments, the snRNA is operably linked to a U7 promoter or a U1 promoter. In some embodiments, the snRNA is operably linked to a U7 promoter and a U1 promoter.

In some embodiments, the snRNA is operably linked to an snRNA downstream terminator (DT).

The disclosure provides a vector comprising a snRNA of any embodiment of the disclosure.

In some embodiments, the vector is a viral vector or a non-viral vector. In some embodiments, the viral vector is an AAV vector.

In some embodiments, the vector comprises multiple copies of the snRNA according to any embodiment of the disclosure. In some embodiments, the vector comprises 2, 3 or 4 copies of the snRNA.

In some embodiments, each copy of the multiple copies of the snRNA is separated by a buffer sequence, wherein the buffer sequence is selected from the group consisting of SEQ ID NOS: 42-48.

The disclosure provides a method of targeting one or more target RNAs of interest and blocking, knocking down, editing, or splicing the one or more target RNAs, comprising contacting the snRNA according to any embodiment of the disclosure with a cell comprising the one or more target RNAs.

The disclosure provides a method of editing adenosine (A) to inosine (I) in an RNA molecule of interest comprising contacting the RNA molecule of interest with an snRNA according to any embodiment of the disclosure.

In some embodiments, the A to I edit is performed by an ADAR molecule recruited by the snRNA.

The disclosure provides a method of treating a disease or disorder in a subject in need thereof comprising administering to the subject an RNA-targeting nucleic acid molecule according to any embodiment of the disclosure or an AAV vector according to any embodiment of the disclosure.

In some embodiments, the disease or disorder is Myotonic Dystrophy Type 1, Amyotrophic lateral sclerosis (ALS), or Hurler syndrome.

The disclosure provides an RNA-targeting nucleic acid molecule comprising an snRNA system (snRNA), wherein the snRNA system comprises at least one targeting sequence, an Sm binding domain (SmBD), and an snRNA stem loop, wherein the binding sequence has extensive complementarity to a target RNA.

### BRIEF DESCRIPTION OF THE DRAWINGS

FIG. 1A depicts a reporter assay construct for evaluating snRNA molecules of the disclosure. The nucleic acid comprises mCherry and GFP driven by a CMV promoter where the mCherry and GFP sequences are separated by a linker with a UAG stop codon. As such, when the stop codon remains intact mCherry will be expressed but GFP will not.

FIG. 1B depicts sequence specific editing of the reporter construct via snRNAs of the disclosure. snRNA molecules comprise a target binding sequence (also referred to herein as a targeting sequence, spacer sequence, or RNA-binding sequence) with a single base pair mismatch that is centered on the stop codon. snRNA molecules induce an A to I editing event facilitated by ADAR proteins, thereby removing the stop codon allowing for expression of both mCherry and GFP proteins.

FIG. 2 is a series of fluorescent images depicting treatment of cells comprising the reporter construct with an snRNA molecule of the disclosure that targets the stop codon in FIGS. 1A and 1B. Images depict mcherry, GFP, and a merge of the mCherry and GFP images. Columns of images depict a stop-codon targeted snRNA, a non-targeting snRNA, no snRNA, and untransfected cells.

FIG. 3 is a graph depicting the ratio of GFP fluorescent signal to mCherry fluorescent signal for cells transfected with snRNA molecules of the disclosure.

FIG. 4A is a schematic of an snRNA of the disclosure comprising from 5′ to 3′: an ADAR recruiting domain, a target binding sequence with a single base pair mismatch, an SM binding domain, and an snRNA stem loop.

FIG. 4B is a schematic of an snRNA of the disclosure comprising from 5′ to 3′: a 5′ interaction stabilization domain (5′ ISD), a target binding sequence with a single base pair mismatch, an SM binding domain, and an snRNA stem loop.

FIG. 4C is a schematic of an snRNA of the disclosure bound to a target mRNA with the single base pair mismatch depicted.

FIG. 5A is a schematic of an snRNA of the disclosure comprising from 5′ to 3′: an ADAR recruiting domain, a target binding sequence, a linker sequence, a target binding sequence, a linker sequence, a target binding sequence, a linker, a target binding sequence with a single base pair mismatch, an SM binding domain, and an snRNA stem loop.

FIG. 5B is a schematic of an snRNA of the disclosure comprising from 5′ to 3′: a 5′ ISD, a target binding sequence, a linker sequence, a target binding sequence, a linker sequence, a target binding sequence, a linker, a target binding sequence with a single base pair mismatch, an SM binding domain, and an snRNA stem loop.

FIG. 5C is a schematic of an snRNA depicted in FIG. 5A bound to a target mRNA with the multiple target binding sequences bound to the mRNA with the single base pair mismatch of one of the target binding sequences depicted. The schematic depicts unbound target mRNA sequences between the snRNA-bound sequences.

FIG. 6A shows schematics of snRNA constructs designed to target the SOD1-AUG start codon. Constructs were designed with and without an ADAR-recruiting sequence. Targeting spacers vary in size from 20 to 151 nucleotides. 20 nucleotide spacer constructs contained additional unique recruitment binding spacers targeting the SOD1 gene. All constructs contain a U7 snRNA stem loop.

FIG. 6B-6C show data supporting that the snRNA SOD1 constructs promote A to I editing of the start codon in 293T cells. Editing was enhanced by snRNAs comprising a cytosine mismatch (in place of a uracil) at the target adenosine nucleotide. FIG. 6B provides a representative Sanger sequencing chromatogram of SOD1 start codon analyzed by EditR and accompanying quantification of nucleotide distribution on the bottom. The top row illustrates the target sequence. The bottom four rows demonstrate percent distribution of labelled nucleotide for each column. Sequence shown as anti-sense due to sequencing primer used (reverse). FIG. 6C provides a bar graph illustrating editing efficiency of SOD1-targeting snRNA constructs with or without a mismatch for the target adenosine. Non-targeting and untreated cells were used as controls. Constructs were transfected into 293T cells and harvested 48 hours later by Trizol (Invitrogen). Editing efficiency was quantified using EditR.

FIG. 7A-7B shows that the snRNA SOD1 constructs targeting the SOD1-AUG start codon reduce SOD1 mRNA levels in 293T cells. Reduction in SOD1 expression observed regardless of mismatch. FIG. 7A provides a bar graph of SOD1 mRNA expression as shown by qRT-PCR. 293T cells were transfected with the SOD1-targeting snRNA constructs with or without a mismatch for the target adenosine. Non-targeting and untreated cells were used as controls. Cells were transfected with constructs for 72 hours and harvested by Trizol (Invitrogen). Sample expression was normalized to endogenous GAPDH.

FIG. 7B shows schematics of the snRNA SOD1 constructs.

FIG. 8A-C shows that the snRNA constructs targeting SOD1 AUG-start codon reduces SOD1 protein levels in 293T cells. Reduction in SOD1 expression observed regardless of mismatch. FIG. 8A provides a Western blot image of SOD1 in 293T cells transfected with the SOD1-targeting snRNA constructs for 72 hours and harvested by RIPA buffer. FIG. 8B. provides quantification of FIG. 8A of SOD1 protein levels in 293T cells transfected with the snRNA constructs with or without target adenosine mismatches. Non-targeting and untreated conditions were used as controls. SOD1 bands were normalized to total protein. Sample intensity shown is relative to untreated conditions.

FIG. 8C shows schematics of the snRNA SOD1 constructs.

FIG. 9A-C shows a Hurler Syndrome (HS) model using patient iPSCs and fibroblasts. FIG. 9A shows a schematic of the Hurler Syndrome premature stop codon. FIG. 9B is a Western blot image showing a loss of IDUA enzyme levels in fibroblasts of HS patients compared to Healthy patients. FIG. 9C is a bar graph of IDUA levels in iPSC myotubes from healthy and Hurler Syndrome patients (ELISA). Most MPS (Mucopolysaccharidoses) I-H patients have a premature stop codon mutation in one or both alleles. These patients are unable to synthesize a full-length polypeptide causing a loss of enzyme activity. The two α-L-iduronidase gene (IDUA) premature stop codon mutations, Q70X and W402X, are the most common (70%) mutations in MPS I patients.

FIG. 10 shows a schematic of snRNA-based mechanisms of action targeting IDUA W402X/Exon 9.

FIGS. 11A-11B shows dose-dependent scAAV snRNA-mediated editing of IDUA mRNA in Hurler Syndrome patient derived myotubes (i.e., A05318 treatment of W402X Hurler Syndrome myotubes). FIG. 11A is a Sanger sequencing chromatograph of IDUA exon 9 depicting A>I (A>G) editing in the A05318 treated myotube at 1E5 and 1E6 vg/cell after 7 days. Untreated cells and AAV empty capsids at MOI 1E5 and 1E6 vg/cell serve as negative controls for the assay. FIG. 11B shows a bar graph depicting editing efficiencies as determined by RT-PCR of IDUA followed by Sanger sequencing chromatograms analyzed by EditR. Untreated and AAV empty capsid treated cells serve as negative controls to show lack of edited RNA in this cell line.

FIG. 12A-12C shows snRNA treatment restores IDUA enzyme in the Hurler myotubes (by ELISA). FIG. 12A shows a recombinant IDUA standard curve as read by IDUA ELISA (Thermo Fisher EH247RB). FIG. 12B shows increasing level of IDUA protein level in the supernatant of co-cultures of healthy and untreated Hurler Syndrome patient derived myotubes at 100:0, 35:65, 50:50, 58:42, and 0:100 ratios (by ELISA). FIG. 12C shows a bar graph depicting IDUA protein levels in the supernatant of A05318 treated myotubes at 1E6 vg/cell after 10 days. Untreated cells and AAV empty capsids at 1E6 vg/cell serve as negative controls for the assay. Percent healthy was calculated keeping healthy myotubes as 100%. Untreated and AAV empty capsids treated cells serve as negative controls to show lack of edited RNA in this cell line.

FIG. 13A-13C shows data supporting that scAAV snRNA (A05318) treated Hurler myotubes exhibit reduced GAG buildups. FIG. 13A provides immunofluorescence images showing reduced Perlecan staining 10 days after treatment of myotubes with A05318 at 1E6 vg/cell with Perlecan antibody (Abcam, red). Untreated cells and AAV empty capsids treated cells at 1E6 vg/cell serve as negative controls to show high baseline staining in this cell line. DAPI stain (blue) was used to detect nuclei. FIG. 13B shows a bar graph depicting mean Perlecan-positive puncta in A05318 treated and untreated cells and cells treated with AAV empty capsids. FIG. 13C provides a bar graph showing Perlecan puncta (uM2) frequency distribution in A05318, AAV empty capsid treated myotubes. Untreated and AAV empty capsid treated cells serve as negative controls and show a higher abundance of larger-sized Perlecan puncta.

FIG. 14 illustrates a targeting strategy for editing MBNL1.

FIG. 15 depicts Adenosine to Inosine editing of the MBNL 3′UTR at the miR30 seed binding site. snRNA 1 and snRNA 2 containing antisense sequences to MBNL1 3′UTR at miR30 binding site and with a mismatch to the A of the seed sequence were transfected into HEK cells for 72 hours. RNA was extracted, reverse transcription was performed and cDNA was amplified with PCR. The resulting DNA amplicon was subjected to sanger sequencing. Chromatographs of amplicon were analyzed by EditR (v10 Moriarity Lab)

### DETAILED DESCRIPTION

The disclosure provides gene therapy compositions comprising a new therapeutic RNA-targeting platform comprised of snRNA (snRNA) comprising an RNA binding sequence (also referred to herein as a targeting sequence or spacer sequence) having at least one base-pairing mismatch. In some embodiments, the snRNA further comprises an Adenosine Deaminase That Acts on RNA (ADAR) recruiting domain. As such, herein we show recruitment of endogenous ADAR1 enzymes to promote targeted A to I editing of therapeutically relevant targets. In some embodiments, A to I editing is used to edit AUG start codons to reduce protein expression. In other embodiment, A to I editing is used to edit miRNA binding sites to reduce miRNA binding with the intention of upregulating protein expression. By screening cells expressing disease-relevant targets (e.g., SOD1 (ALS), MBNL1 (DM1), IDUA (Hurler Syndrome), we've identified snRNA targeting and/or recruitment spacers capable of effecting A to I editing via our snRNA platform. snRNA molecules of the disclosure can be non-natural, modified and/or engineered snRNA (esnRNA). esnRNA molecules comprising the disclosed editing and/or recruitment spacers have a mutated snRNA stem loop. In some embodiments, the snRNA molecules comprising the disclosed editing and/or recruitment spacers have a native stem loop.

Disclosed herein are compositions comprising nucleic acid molecules, and vectors comprising the snRNA (snRNA) editing and/or recruitment systems.

Small Nuclear Ribonucleic Acids (snRNAs)

Small nuclear ribonucleic acids (snRNAs) are essential components of small nuclear ribonucleoprotein complexes (snRNPs) which, when assembled with additional proteins, form the large ribonucleoprotein complex known as the spliceosome, the cell machinery appointed to mediate the entire mRNA maturation process. The spliceosome is responsible for precursor mRNA splicing; the process that removes introns from RNA transcripts before protein production. An individual snRNA is generally about 250 nucleotides or less in size. For example, U1 snRNA is 164 nucleotides in length and is encoded by genes that occur in several copies within the human genome. U1 snRNA represents the ribonucleic component of the nuclear particle U1 snRNP. The Ut snRNA has a stem and loop tridimensional structure and within the 5′ region there is a single-stranded sequence, generally about 9 nucleotides in length, capable of binding by complementary base pairing to the splicing donor site on the pre-mRNA molecule. (Horowitz et al., 1994, Trends Genet., 10(3):100-6.) The various spliceosomal snRNAs have been designated as U1, U2, U4, U5, U6, U4ATAC, U6ATAC, U7, UT 1 and U12, due to the generous amount of uridylic acid they contain. (Mattaj et al., 1993, FASEB J, 15, 7:47-53)

The snRNA systems of the disclosure can be used for treating G to A point mutations and/or other toxic mutations. For example, antisense oligonucleotides that interfere with splice sites and regulatory elements within an exon containing toxic mutations, induce skipping of specific exons at the pre-mRNA level. Such antisense sequences can be delivered using viral vectors carrying a gene from which the antisense sequence comprised within an snRNA molecule of the disclosure can be transcribed. U7 snRNA is endogenously involved in histone pre-mRNA 3′-end processing, but can be converted into a versatile tool for splicing modulation by a small change in the binding site for Sm/Lsm proteins.

Most U-rich snRNPs are complexes that mediate the splicing of pre-mRNAs. U7 snRNP is an exception. U7 is not involved in splicing but rather is a key factor in the unique 3′-end processing of replication-dependent histone mRNAs. By modifying the U7 snRNA histone binding sequence and the Sm motif, U7 can no longer be involved in processing the histone pre-mRNA and instead targets pre-mRNAs or mRNA for editing of one or more bases in an RNA sequence. In this manner. U7 snRNA can be used as an effective gene therapy platform. A U7 snRNA platform also has the additional advantages of being a compact size, having the capability to accumulate in the nucleus without causing cellular toxicity, and possesses little to no immunoreactivity. (Gadgil et al., 2021, J Gene Med, 23(4): e3321.)

### Adenosine Deaminase That Acts on RNAs (ADAR)

Adenosine Deaminase That Acts on RNAs (ADAR) are a class of RNA-binding enzymes that convert adenosine (A) to inosine (I) via deamination. The resultant inosine pairs with cytosine and is recognized as guanine by translational machinery. In mammals, there are three types of ADAR enzymes. ADAR1. ADARB1 (ADAR2) and ADARB2 (ADAR3). ADAR1 and ADARB1 are found in many tissues in the body while ADARB2 is only found in the brain. ADAR1 and ADARB1 are known to be catalytically active while evidence suggests ADARB2 is inactive. ADAR1 has two known isoforms, ADAR1p150 and ADAR1p110. ADAR1p110 is typically found in the nucleus while ADAR1p150 shuffles between the nucleus and the cytoplasm, mostly present in the cytoplasm. ADAR1 and ADARB1 share functional domains and have similar expression patterns, structure of proteins, and require substrate double stranded RNA structures. However, they differ in their editing activity.

### ADAR Recruiting Domains

ADARs from all characterized species have a modular domain organization consisting of one-to-three double-stranded RNA binding domains (dsRBM) followed by a conserved C-terminal catalytic adenosine deaminase domain. ADARs recognize and bind to short nucleic acids sequences, ADAR recruiting domains. The snRNAs of the disclosure can comprise ADAR recruiting domains. The ADAR recruiting domain binds to the dsRBD1 and dsRBD2 domains of ADAR2, and then the deaminase domain of ADAR2 binds to the A-C mismatch. In some embodiments, the ADAR recruiting domain comprises, consists essentially of, or consists of a nucleic acid sequence at least 65%, 70%, 75%, 80%, 85%, 90%, 95%, 96%, 97%, 98%, 99% or 100% (or any percentage in between) identical to

(SEQ ID NO: 88)
GGTGTCGAGAAGAGGAGAACAATATGCTAAATGTTGTTC
TCGTCTCCTCGACACC.

Without wishing to be bound by theory, ADAR recruiting domains of snRNAs of the disclosure are bound by ADAR proteins bringing them into proximity and the proper position such that the catalytic adenosine deaminase domain can act on the adenosine to be edited. See Wettengel et al., Nucleic Acids Res. 45, 2797-2808 (2017). See also Stefl et al., Cell. 2010; 143:225-237.

snRNAs

Disclosed herein is an snRNA platform comprising an RNA binding sequence (also referred to herein as a targeting sequence or spacer sequence) that has extensive complementarity and/or at least one base-pairing mismatch. snRNAs of the disclosure can further comprise an Sm binding domain and snRNA stem loop. Extensive complementarity would be no less than 30 consecutive nucleotides of complementarity or between about 30 and about 200 consecutive nucleotides of complementarity. In some embodiments, extensive complementarity would be at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 nucleotides of consecutive complementarity. In some embodiments, the extensive complementarity includes at least one base-pairing mismatch. In some embodiments, the extensive complementarity includes exactly one base-pairing mismatch. In some embodiments, the base-pairing mismatch occurs at the adenosine to be edited on the target RNA sequence.

In some embodiments, the snRNA stem loop is an engineered stem loop. In some embodiments, the snRNA stem loop is a native snRNA stem loop. In some embodiments, snRNAs of the disclosure further comprise an Adenosine Deaminase That Acts on RNA (ADAR) recruiting domain.

### Engineered Stem Loops

In some embodiments, snRNA molecules disclosed herein can comprise a native snRNA stem loop. In some embodiments, snRNA molecules disclosed herein can comprise an engineered stem loop (eSL) which includes compensatory modifications to a native snRNA stem loop. Compensatory modifications made to the native stem loop sequence create an engineered stem loop (eSL) which more effectively communicates (folds and anneals) with the snRNA interaction stabilization domain (ISD) which in turn creates a snRNA platform with increased stability. U7 snRNAs have been previously shown to be programmable to modulate mRNAs. Disclosed herein are programmed snRNA improvements which are capable of being used as a gene therapy tool. In one embodiment, these snRNA are human snRNAs. In one embodiment the U7 snRNA is a human U7 snRNA. In another embodiment disclosed herein, snRNA comprises varying types of snRNAs (U1-U12, etc.) by combining domains of endogenous snRNAs to fine tune stabilization of the platform and/or to reduce off-target effects. For example, in one embodiment, the engineered snRNA system comprises a combination of human U7 and human U1 snRNA components.

Additional elements that can tune the processing and abundance of the RNA can be further engineered into the snRNAs comprising eSLs. In one embodiment, additional elements that can tune the processing, stability, and abundance of the snRNA can be further engineered into the snRNAs at the 5′ or 3′ ends. In another embodiment, such elements may include but are not limited to stem loops, hairpins, G-C clamps, kissing loops, triplexes, quadruplexes, and protein binding sites. Engineered stem loops are described in WO2023168458, the contents of which are incorporated herein by reference in its entirety for examples of eSL sequences that may be used in the constructs described herein.

The snRNA (snRNA) system disclosed herein can comprise an engineered stem loop (eSL) which includes compensatory modifications to a native snRNA stem loop. These modifications result in increased stability of the snRNA compared to snRNA comprising an unmodified stem loop. An eSL disclosed herein can be derived from any snRNP U1-U12. In one embodiment, the eSL is a U7 eSL. In another embodiment, a human eSL comprises, consists essentially of, or consists of a nucleic acid sequence at least 65%, 70%, 75%, 80%, 85%, 90%, 95%, 96%, 97%, 98%, 99% or 100% (or any percentage in between) identical to the following nucleotide sequences:

(SEQ ID NO: 1)
ggctttctggctccttaccggaaagcc,
(SEQ ID NO: 2)
ggctttctgggaggttaccggaaagcc,
(SEQ ID NO: 3)
ggctttctggcctccttaccggaaagcc,
(SEQ ID NO: 4)
ggctttctggggaggttaccggaaagcc,
(SEQ ID NO: 5)
ggctttctggctggctaccggaaagcc,
(SEQ ID NO: 6)
ggctttctggcttccccggaaagcc,
(SEQ ID NO: 7)
ggctttctggcttcttcccggaaagcc,
(SEQ ID NO: 8)
ggctttctggcaacttaccggaaagcc,
(SEQ ID NO: 9)
ggctttctggttcggtaccggaaagcc,
(SEQ ID NO: 10)
ggctttctggaagccttaccggaaagcc
(SEQ ID NO: 11)
ggctttctggcttcttaccggaaagcc,
or
(SEQ ID NO: 12)
GGCTTTCTGGCCTCCGCCGGAAAGCCCCT.

In some embodiments, a human eSL comprises the sequence set forth in SEQ ID NO: 1. In some embodiments, a human eSL comprises the sequence set forth in SEQ ID NO: 2. In some embodiments, a human eSL comprises the sequence set forth in SEQ ID NO: 3. In some embodiments, a human eSL comprises the sequence set forth in SEQ ID NO: 4. In some embodiments, a human eSL comprises the sequence set forth in SEQ ID NO: 5. In some embodiments, a human eSL comprises the sequence set forth in SEQ ID NO: 6. In some embodiments, a human eSL comprises the sequence set forth in SEQ ID NO: 7. In some embodiments, a human eSL comprises the sequence set forth in SEQ ID NO: 8. In some embodiments, a human eSL comprises the sequence set forth in SEQ ID NO: 9. In some embodiments, a human eSL comprises the sequence set forth in SEQ ID NO: 10. In some embodiments, a human eSL comprises the sequence set forth in SEQ ID NO: 11. In some embodiments, a human eSL comprises the sequence set forth in SEQ ID NO: 12.

In some embodiments, a murine eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 13)
ggctttctggctccttaccggaaagcccct
(SEQ ID NO: 14)
Ggttttctgacctccgtcggaaaacccct,
(SEQ ID NO: 15)
ggttttctgacctccttcggtcggaaaacccct,
(SEQ ID NO: 16)
Ggttttctgacctccgtcggaaaacc,
(SEQ ID NO: 17)
GGTTTTCTGACACTCCGTCGGAAAACCCCT,
(SEQ ID NO: 18)
GGTTTTCTGATCTCCATCGGAAAACCCCT,
or
(SEQ ID NO: 19)
GGTTTTCCGACCTCCGTCGGAAAACCCCT.

In some embodiments, a murine eSL comprises the sequence set forth in SEQ ID NO: 13. In some embodiments, a murine eSL comprises the sequence set forth in SEQ ID NO: 14. In some embodiments, a murine eSL comprises the sequence set forth in SEQ ID NO: 15. In some embodiments, a murine eSL comprises the sequence set forth in SEQ ID NO: 16. In some embodiments, a murine eSL comprises the sequence set forth in SEQ ID NO: 17. In some embodiments, a murine eSL comprises the sequence set forth in SEQ ID NO: 18. In some embodiments, a murine eSL comprises the sequence set forth in SEQ ID NO: 19.

In some embodiments, a human or murine eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 20)
GGCTTTCTGGCACTCCACCGGAAAGCCCCT,
(SEQ ID NO: 21)
GGCTTTCTGGCACTCCGCCGGAAAGCCCCT,
or
(SEQ ID NO: 22)
GGCTTTCTGGCCTCCACCGGAAAGCCCCT.

In some embodiments, a human or murine eSL comprises the sequence set forth in SEQ IID NO: 20. In some embodiments, a human or murine eSL comprises the sequence set forth in SEQ IID NO: 21. In some embodiments, a human or murine eSL comprises the sequence set forth in SEQ IID NO: 22.

In some embodiments, a dog or cat eSL comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleotide sequence

(SEQ ID NO: 23)
GGTTTTCCGGTCTCCACCGGAAAGCCCCC

In some embodiments, a cow, sheep, or goat eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 24)
GGCTTTCCGGTCTCCACCGGAAAGCCCCT,
or
(SEQ ID NO: 25)
GGCTTTCCGGCCTCCGCCGGAAAGCCCCT.

In some embodiments, a cow, sheep, or goat eSL comprises the sequence set forth in SEQ ID NO: 24. In some embodiments, a cow, sheep, or goat eSL comprises the sequence set forth in SEQ ID NO: 25.

In some embodiments, a pig eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 26)
GGTTTTCCGGTCTCCACCGGAAAACCCTT,
(SEQ ID NO: 27)
GGTTTTCCGTGCTCCCACGGAAAACCCTT,
(SEQ ID NO: 28)
GGTTTTCCGGCCTCCGCCGGAAAACCCTT,
(SEQ ID NO: 29)
GGTTTTCCGTGACTCCCACGGAAAACCCTT,
or
(SEQ ID NO: 30)
GGTTTTCCGGCACTCCGCCGGAAAACCCTT

In some embodiments, a pig eSL comprises the sequence set forth in SEQ ID NO: 26. In some embodiments, a pig eSL comprises the sequence set forth in SEQ ID NO: 27. In some embodiments, a pig eSL comprises the sequence set forth in SEQ ID NO: 28. In some embodiments, a pig eSL comprises the sequence set forth in SEQ ID NO: 29. In some embodiments, a pig eSL comprises the sequence set forth in SEQ ID NO: 30, 10471 In some embodiments, a horse eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 31)
GGTCTTCCGGTCTCCTCCGGAAGGCCCCC,
or
(SEQ ID NO: 32)
GGTCTTCCGGCTCCCCGGAAGGCCCCC.

In some embodiments, a horse eSL comprises the sequence set forth in SEQ ID NO: 31. In some embodiments, a horse eSL comprises the sequence set forth in SEQ ID NO: 32.

In some embodiments, a sheep eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 33)
GGCTTTCCGTGCTCCCACGGAAAGCCCCT,
(SEQ ID NO: 34)
GGCTTTCCGTGACTCCCACGGAAAGCCCCT,
or
(SEQ ID NO: 35)
GGCTTTCCGGCACTCCGCCGGAAAGCCCCT.

In some embodiments, a sheep eSL comprises the sequence set forth in SEQ ID NO: 33. In some embodiments, a sheep eSL comprises the sequence set forth in SEQ ID NO: 34. In some embodiments, a sheep eSL comprises the sequence set forth in SEQ ID NO: 35.

In some embodiments, snRNA molecules of the disclosure comprise a native snRNA stem loop comprises the sequence set forth in SEQ ID NO: 36. In some embodiments is a native snRNA stem loop comprises the sequence set forth in SEQ ID NO: 37. In some embodiments is a native snRNA stem loop comprises the sequence set forth in SEQ ID NO: 38. In some embodiments is a native snRNA stem loop comprises the sequence set forth in SEQ ID NO: 39.

In some embodiments, engineered stem loops provide for enhanced stability of an snRNA relative to an snRNA comprising a native stem loop. In some embodiments is a native snRNA stem loop comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 36)
Ggttttctgacttcggtcggaaaacccct,
(SEQ ID NO: 37)
ggttttctgacttcggtcggaaaacc,
(SEQ ID NO: 38)
Ggctttctggctttttaccggaaagcc,
(SEQ ID NO: 39)
ggctttctggctttttaccggaaagccCCT,
(SEQ ID NO: 40)
GGCTTTCCGGCCTCCGCCGGAAAGCCCCT,
or
(SEQ ID NO: 41)
GGCTTTCCGGCCTCCGCCGGAAAGCC.

### 5′ Interaction Stability Domains

The eSL disclosed herein possesses more effective folding and annealing properties with a 5′ interaction stability domain (5′ISD) and this in turn results in increased stability of the esnRNA compared to a non-engineered snRNA. The 5′ ISD has nucleotides that are complementary to the nucleotides within the engineered SL, and without wishing to be bound by theory, an interaction between the 5′ISD and eSL is predicted to form secondary structure that protects the 5′ end of an snRNA. In some embodiments the 5′ ISD anneals and/or hybridizes to an eSL of the disclosure. In some embodiments the 5′ISD is a sequence having complementarity and/or reverse complementarity to a sequence present in an eSL of the disclosure. In some embodiments a 5′ISD disclosed herein can comprise or consist of one of the following nucleotide sequences:

ggagt,
cctct,
ggaggt
cctcct,
agccag,
ggaag,
gaagaag,
gttg,
ccgaa,
taaggag,
gaag,
and
ggctt.

### RNA Targeting Sequences

The snRNA systems can be programmed to comprise one or more RNA targeting sequence (TS) (also termed “spacer sequences”) that target one or more RNAs of interest (also termed spacers). In one example, U7 snRNA can be programmed by replacing the histone mRNA binding sequence with a sequence complementary to a target of interest.

In some embodiments, the targeting sequence binds an mRNA or pre-mRNA sequence. In some embodiments, at least one targeting sequence of the disclosure comprises at least one base-pairing mismatch. In some embodiments, the base-pairing mismatch is an adenosine (A)-cytosine (C) mismatch. In some embodiments, the adenosine is on the mRNA or pre-mRNA sequence target sequence and the cytosine is located on the binding sequence of the snRNA.

Targeting sequences of the disclosure can bind (target) any sequence or region of an mRNA or pre-mRNA sequence. In some embodiments, targeting sequences of the disclosure bind a start codon, a stop codon, or a splicing regulatory sequence. In some embodiments, the splicing regulatory sequence is a branch point adenosine (bpA) sequence, 3′ acceptor splice site (3′ ss) sequence, 5′ ss (beyond GU), or exonic splicing enhancer (ESE) sequence. In some embodiments, targeting sequences of the disclosure bind intronic or exonic sequences of pre-mRNA. Targeting sequences of the disclosure comprise either extensive complementarity and intra or inter molecular base-pairing and/or base-pair mismatch between an adenosine on the target RNA sequence and a cytosine on the snRNA targeting sequence. This mismatch enables adenosine deamination by an ADAR thereby converting adenosine to inosine. Inosine is recognized by translational machinery as a guanine. In the aspect of extensive complementarity of greater than about 30 nucleotides, the RNA binding sequence of the disclosure comprises a targeting sequence of about greater than 30 nucleotides with or without a base-pair mismatch between an adenosine on the target RNA sequence and a cytosine on the snRNA binding sequence (also referred to as a targeting sequence). As such, when an A to I editing event is initiated by snRNA molecules of the disclosure, the resulting target RNA harboring the inosine will be recognized as harboring a guanine in place of the adenosine. In some embodiments, guanine to adenosine point mutations can be corrected using the snRNA molecules of the disclosure. In some embodiments, an A to I editing event can result in a single amino acid mutation in an expressed protein of interest. An A to I editing event can remove a stop codon in an mRNA sequence. In some embodiments, the stop codon is a premature stop codon. Thus, removal of said premature stop codon can lead to rescued translation of a protein of interest.

In some embodiments, targeting sequences of the disclosure comprise at least one base-pair mismatch and are at least about 1, at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 nucleotides in length. In some embodiments, targeting sequences of the disclosure are at least about 10, 20, 30, 40, 50, 60, or about 70 nucleotides in length.

In some embodiments, targeting sequences of the disclosure comprise extensive complementarity without a base-pair mismatch and are at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 nucleotides in length.

In some embodiments, the editing efficiency is correlated with targeting sequence length.

In some embodiments, targeting sequences of the disclosure comprise full sequence complementarity to the target RNA sequence with the exception being the A-C mismatch at the site of the adenosine to be edited.

Targeting sequences of the disclosure can comprise, consist essentially of, or consist of a nucleic acid sequence at least 65%, 70%, 75%, 80%, 85%, 90%, 95%, 96%, 97%, 98%, 99% or 100% (or any percentage in between) identical to the following nucleic acid sequences:

(SEQ ID NO: 120)
tgaacagctcctcgcccttgctcactggcagagccctcCagcatc
gcgagcaggcgctgcctcctccgcc;
(SEQ ID NO: 121)
tcgccgtccagctcgaccaggatgggcaccaccccggtgaacagc
tcctcgcccttgctcactggcagagccctcCagcatcgcgagcag
gcgctgcctcctccgccgctgcctcctccgccgctgcctcctccg
ccctgcagcttgtaca;
(SEQ ID NO: 122)
tgaacagctcctcgcccttgctcactggcagagccctcTagcatc
gcgagcaggcgctgcctcctccgcc;
(SEQ ID NO: 123)
tcgccgtccagctcgaccaggatgggcaccaccccggtgaacagc
tcctcgcccttgctcactggcagagccctcTagcatcgcgagcag
gcgctgcctcctccgccgctgcctcctccgccgctgcctcctccg
ccctgcagcttgtaca

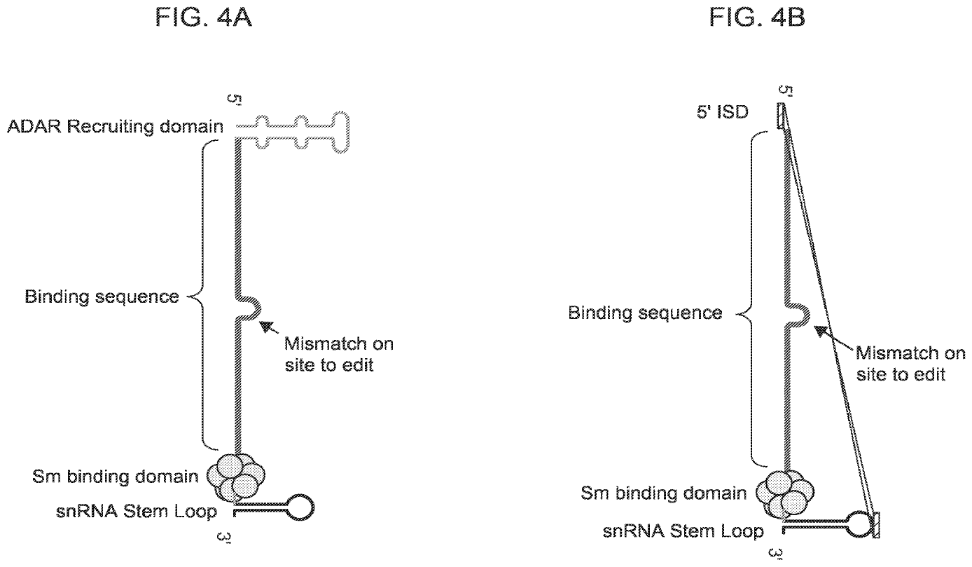

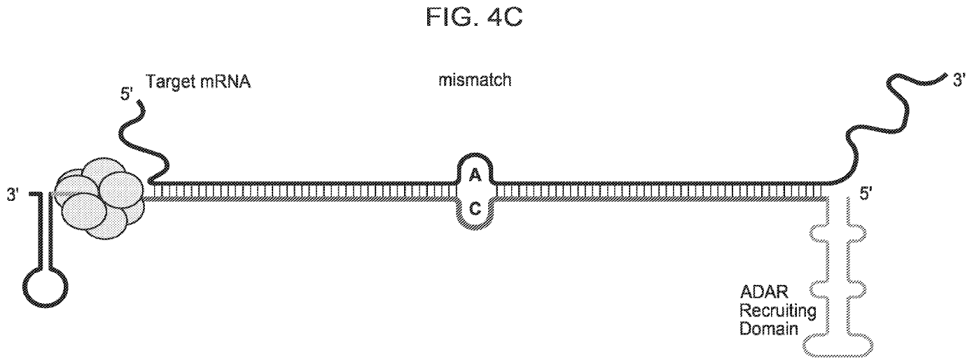

Exemplary snRNA molecules comprising base-pair mismatch targeting sequences are depicted in FIGS. 4A and 4B. FIG. 4C depicts exemplary snRNA bound to a target mRNA sequence. The binding site comprises an A-C mismatch at the site of adenosine editing.

The snRNA molecules of the disclosure can comprise one or more additional targeting sequences that bind to target RNA sequences of the disclosure. In some embodiments, the one or more additional targeting sequences comprise full sequence complementarity to the target RNA sequence. In some embodiments, the one or more additional targeting sequences can comprise a base-pair mismatch.

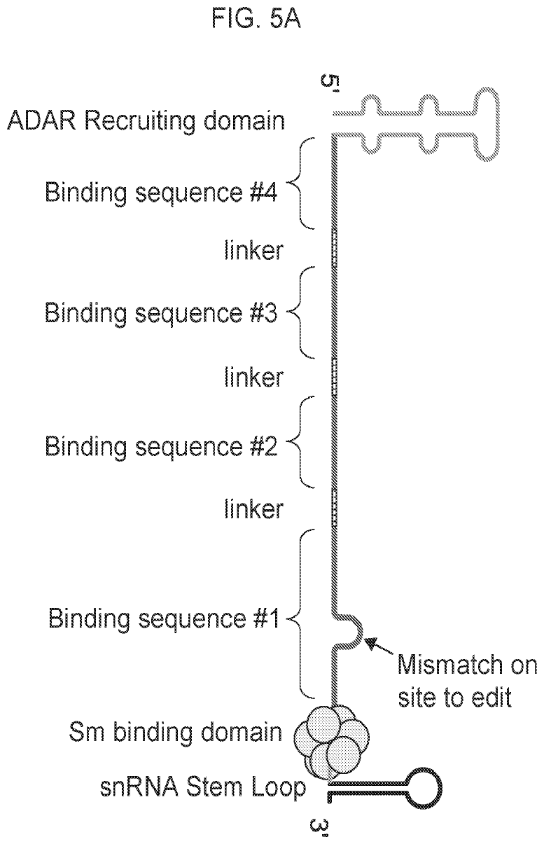

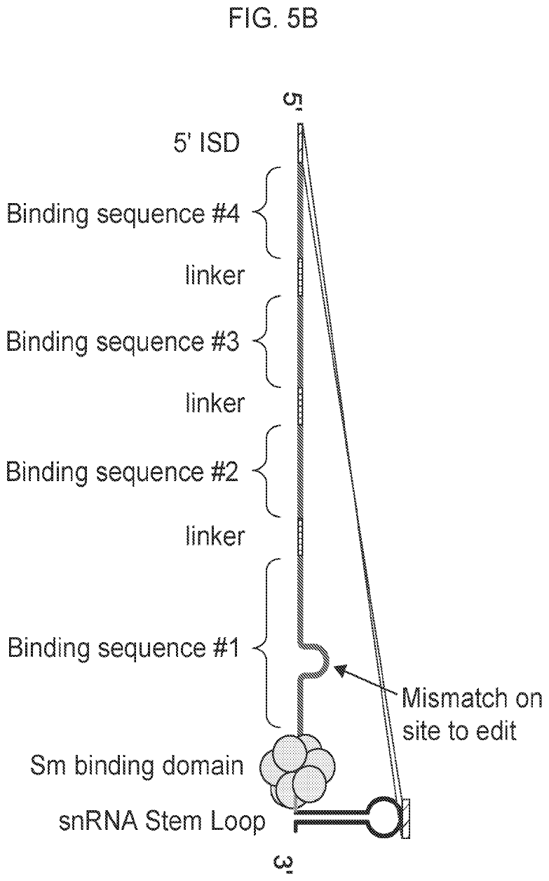

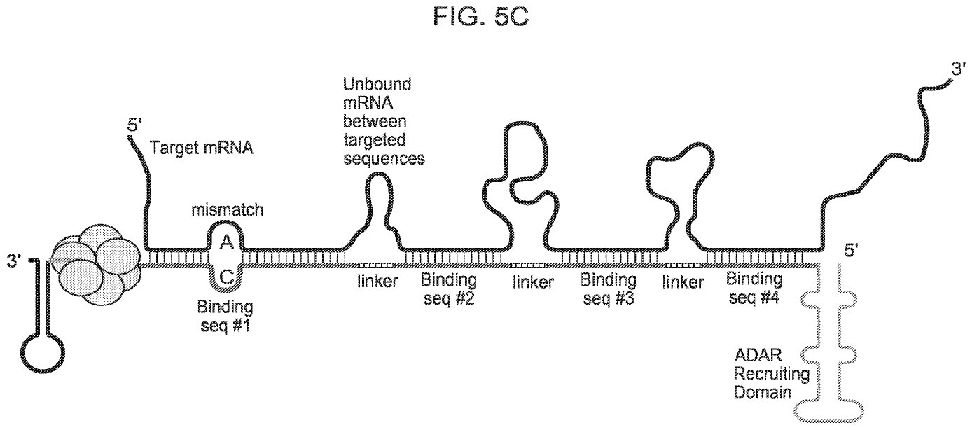

Additional targeting sequences of the disclosure can be utilized to confer enhanced binding affinity or binding specificity of the snRNA to the target RNA. In some embodiments. snRNA of the disclosure comprise at least one, at least two, at least three, at least 4, at least five, at least six, at least seven, or at least eight additional targeting sequences. In some embodiments, nucleic acid linker sequences are used to separate binding sites. Exemplary snRNA molecules comprising additional targeting sequences are depicted in FIGS. 5A and 5B. FIG. 5C depicts multi-binding site snRNA bound to a target mRNA sequence. Binding site 1 comprises an A-C mismatch at the site of adenosine editing. Binding sites 2-4 do not comprise a base-pair mismatch.

The disclosure provides snRNA molecules capable of binding RNA sequences encoding SOD1. Accordingly, the disclosure provides SOD1 targeting sequences. Targeting sequence that binds SOD1 can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in the Table which follows:

SOD1 targeting sequence

CCGTCGCCCTTCAGCACGCACACGGCCTTCGTCGCCATAACTCGCTAGG
CCACGCCGAGGTCCTGGTTCC
(SEQ ID NO: 124)
CCGTCGCCCTTCAGCACGCACACGGCCTTCGTCGCCAcAACTCGCTAGG
CCACGCCGAGGTCCTGGTTCC
(SEQ ID NO: 125)
ATGATGCCCTGCACTGGGCCGTCGCCCTTCAGCACGCACACGGCCTTCG
TCGCCATAACTCGCTAGGCCACGCCGAGGTCCTGGTTCCGAGGACTGCA
ACGGAAACCCCAG
(SEQ ID NO: 126)
ATGATGCCCTGCACTGGGCCGTCGCCCTTCAGCACGCACACGGCCTTCG
TCGCCAcAACTCGCTAGGCCACGCCGAGGTCCTGGTTCCGAGGACTGCA
ACGGAAACCCCAG
(SEQ ID NO: 127)
TCCTTCTGCTCGAAATTGATGATGCCCTGCACTGGGCCGTCGCCCTTCA
GCACGCACACGGCCTTCGTCGCCATAACTCGCTAGGCCACGCCGAGGTC
CTGGTTCCGAGGACTGCAACGGAAACCCCAGACGCTGCAGGAGACTACG
ACGC(SEQ ID NO: 128)
GTCGCCATAACTCGCTAGGCaaaCGCCGAGGTCCTGGTaaaGCAACGGA
AACCCCAaaaGCAGGAGACTACGAC
(SEQ ID NO: 129)
GTCGCCAcAACTCGCTAGGCaaaCGCCGAGGTCCTGGTaaaGCAACGGA
AACCCCAaaaGCAGGAGACTACGAC
(SEQ ID NO: 130)
GCCCTGCACTGGGCCaaaAGCACGCACACGGCCaaaGTCGCCATAACTC
GCTAGGCaaaGCAACGGAAACCCCA
(SEQ ID NO: 131)
GCCCTGCACTGGGCCaaaAGCACGCACACGGCCaaaGTCGCCAcAACTC
GCTAGGCaaaGCAACGGAAACCCCA
(SEQ ID NO: 132)
GTCGCCCTTCAGCACGCACACGGCCTTCGTCGCCATAACTCGCTAGGCC
ACGCCGAGGTCCTGGTTCCGAG
(SEQ ID NO: 133)
GTCGCCCTTCAGCACGCACACGGCCTTCGTCGCCAcAACTCGCTAGGCC
ACGCCGAGGTCCTGGTTCCGAG
(SEQ ID NO: 134)
GCCCTGCACTGGGCCaaaAGCACGCACACGGCCaaaGTCGCCAcAACTC
GCTAGGCCACGCCGAGGTCCTGGTTCCaaaGCAACGGAAACCCCA
(SEQ ID NO: 135)
CGTCGCCACAACTCGCTAGGCCACGCCGAGGTCCTGGTTCCGAGGAC
(SEQ ID NO: 136)
GCACTGGGCCGTCGCCCTTCAGCACGCACACGGCCTTCGTCGCCAcAAC
TCGCTAGGCCACGCCGAGGTCC
(SEQ ID NO: 137)
ATGATGCCCTGCACTGGGCCGTCGCCCTTCAGCACGCACACGGCCTTCG
TCGCCAcAACTCGCTAGGCCAC
(SEQ ID NO: 138)
CTCGAAATTGATGATGCCCTGCACTGGGCCGTCGCCCTTCAGCACGCAC
ACGGCCTTCGTCGCCAcAACTC
(SEQ ID NO: 139)
TTTCCTTCTGCTCGAAATTGATGATGCCCTGCACTGGGCCGTCGCCCTT
CAGCACGCACACGGCCTTCGTCGCCAcAACTCGCTAGGCCACGCCGAGG
TCCTGGTTCCGAG
(SEQ ID NO: 140)
GGTCCATTACTTTCCTTCTGCTCGAAATTGATGATGCCCTGCACTGGGC
CGTCGCCCTTCAGCACGCACACGGCCTTCGTCGCCAcAACTCGCTAGGC
CACGCCGAGGTCC
(SEQ ID NO: 141)
CACCTTCACTGGTCCATTACTTTCCTTCTGCTCGAAATTGATGATGCCC
TGCACTGGGCCGTCGCCCTTCAGCACGCACACGGCCTTCGTCGCCAcAA
CTCGCTAGGCCAC
(SEQ ID NO: 142)
TCCATTACTTTCCTTCTGCTCGAAATTGATGATGCCCTGCACTGGGCCG
TCGCCCTTCAGCACGCACACGGCCTTCGTCGCCAcAACTCGCTAGGCCA
CGCCGAGGTCCTGGTTCCGAGGACTGCAACGGAAACCCCAGACGCTGCA
GGAG
(SEQ ID NO: 143)
CCTTCACTGGTCCATTACTTTCCTTCTGCTCGAAATTGATGATGCCCTG
CACTGGGCCGTCGCCCTTCAGCACGCACACGGCCTTCGTCGCCAcAACT
CGCTAGGCCACGCCGAGGTCCTGGTTCCGAGGACTGCAACGGAAACCCC
AGAC
(SEQ ID NO: 144)
CTTCCCCACACCTTCACTGGTCCATTACTTTCCTTCTGCTCGAAATTGA
TGATGCCCTGCACTGGGCCGTCGCCCTTCAGCACGCACACGGCCTTCGT
CGCCAcAACTCGCTAGGCCACGCCGAGGTCCTGGTTCCGAGGACTGCAA
CGGA
(SEQ ID NO: 145)
TCCTCGGAACCAGGACCTCGGCGTGGCCTAGCGAGTTgTGGCGACGAAG
GCCGTGTGCGTGCTGAAGGGCGACGGCCCAGTGCAGGGCATCATCAATT
TCGAGCAGAAGGAAAGTAATGGACCAGTGAAGGTGTGGGGAAGCATTAA
AGGA
(SEQ ID NO: 146)
CTTCAGTCAGTCCTTTAATGCTTCCCCACACCTTCACTGGTCCATTACT
TTCCTTCTGCTCGAAATTGATGATGCCCTGCACTGGGCCGTCGCCCTTC
AGCACGCACACGGCCTTCGTCGCCAcAACTCGCTAGGCCACGCCGAGGT
CCTG
(SEQ ID NO: 147)
GCCCTGCACTGGGCCGaaaCAGCACGCACACGGCCaaaGTCGCCAcAAC
TCGCTAGGCaaaGCAACGGAAACCCCAGACGC
(SEQ ID NO: 148)
GCCCTGCACTGGGCCGaaaCAGCACGCACACGGCCaaaGTCGCCAcAAC
TCGCTAGGCCACGCCGAGGTCCTGGTTCCaaaGCAACGGAAACCCCAGA
CGC
(SEQ ID NO: 149)
GCCCTGCACTGGGCCGaaaCAGCACGCACACGGCCaaaGTCGCCAcAAC
TCGCTAGGCCACGCCGAGGTCCTGGTTCCGAGGACaaaGCAACGGAAAC
CCCAGACGC
(SEQ ID NO: 150)

The disclosure provides snRNA molecules capable of binding RNA sequences encoding IDUA. Accordingly, the disclosure provides IDUA targeting sequences. Targeting sequence that binds IDUA can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in the Table 1, which follows:

IDUA Targeting Sequence

GCCCACCGTGTGGTTGCTGTCCAGGACGGTCCCGGCCTGCGACAC
TTCGGCCcAGAGCTGCTCCTCATCCAGCAGCGCCAGCAGCCCCAT
GGCCGTGAGCACCGG
TTCGGCCcAGAGCTGCTCCTAAAGGCCGTGAGCACCGGAAACGTG
CGGCGGGCGGGAAAGCGCGAAGGGGTGCGGGTGG

The disclosure provides snRNA molecules capable of binding RNA sequences encoding MBNL1. Accordingly, the disclosure provides MBNL1 targeting sequences. Targeting sequence that binds MBNL1 can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in the Table, which follows:

MBNL1 Targeting Sequence

tggcaattttaaatacaaggtgcacctttgttatttgCaaaccttgaaa
gaaataaacttatttttaaaaa (SEQ ID NO: 156)
tggcaattttaaatacaaggtgcacctttgttattCgtaaaccttgaaa
gaaataaacttatttttaaaaa (SEQ ID NO: 157)
tggcaattttaaatacaaggtgcacctttgttattCgCaaaccttgaaa
gaaataaacttatttttaaaaa (SEQ ID NO: 158)
ttaaacaaaaatgactgtgcatgcacgctctcatctataatggcaattt
taaatacaaggtgcacctttgttatttgCaaaccttgaaagaaataaac
ttatttttaaaaaattatatcttggtctacagacgtaccaatacataat
agaa
(SEQ ID NO: 159)
ttaaacaaaaatgactgtgcatgcacgctctcatctataatggcaattt
taaatacaaggtgcacctttgttattCgtaaaccttgaaag
aaataaacttatttttaaaaaattatatcttggtctacagacgtaccaa
tacataatagaa (SEQ ID NO: 160)
ttaaacaaaaatgactgtgcatgcacgctctcatctataatggcaattt
taaatacaaggtgcacctttgttattCgCaaaccttgaaa
gaaataaacttatttttaaaaaattatatcttggtctacagacgtacca
atacataatagaa (SEQ ID NO: 161)
tggatacactaattcttttaaacaagtgcccattaCcacattttgcatt
tttgagtgcttctgagttgaaa (SEQ ID NO: 162)
tggatacactaattcttttaaacaagtgcccattatcacaCtttgcatt
tttgagtgcttctgagttgaaa (SEQ ID NO: 163)
tggatacactaattcttttaaacaagtgcccattatcacatCttgcatt
tttgagtgcttctgagttgaaa (SEQ ID NO: 164)
tggatacactaattcttttaaacaagtgcccattaCcacaCCttgcatt
tttgagtgcttctgagttgaaa (SEQ ID NO: 165)
taaccagctggagtgaaggctggatacactaattcttttaaacaagtgc
ccattaCcacattttgcatttttgagtgcttctgagttgaaaattaaaa
ccatgaaatttga
(SEQ ID NO: 166)
taaccagctggagtgaaggctggatacactaattcttttaaacaagtgc
ccattatcacaCtttgcatttttgagtgcttctgagttgaaaattaaaa
ccatgaaatttga
(SEQ ID NO: 167)
taaccagctggagtgaaggctggatacactaattcttttaaacaagtgc
ccattatcacatCttgcatttttgagtgcttctgagttgaa
aattaaaaccatgaaatttga (SEQ ID NO: 168)
taaccagctggagtgaaggctggatacactaattcttttaaacaagtgc
ccattaCcacaCCttgcatttttgagtgcttctgagttgaaaattaaaa
ccatgaaatttga
(SEQ ID NO: 169)
gctgataagtgcaacatttttaaccagctggagtgaaggctggatacac
taattcttttaaacaagtgcccattaCcacattttgcatttttgagtgc
ttctgagttgaaaattaaaaccatgaaatttgaactgcgttttattttt
cacc
(SEQ ID NO: 170)
gctgataagtgcaacatttttaaccagctggagtgaaggctggatacac
taattcttttaaacaagtgcccattatcacaCtttgcatttttgagtgc
ttctgagttgaaaattaaaaccatgaaatttgaactgcgttttattttt
cacc
(SEQ ID NO: 171)
gctgataagtgcaacatttttaaccagctggagtgaaggctggatacac
taattcttttaaacaagtgcccattatcacatCttgcatttttgagtgc
ttctgagttgaaaattaaaaccatgaaatttgaactgcgttttattttt
cacc
(SEQ ID NO: 172)
gctgataagtgcaacatttttaaccagctggagtgaaggctggatacac
taattcttttaaacaagtgcccattaCcacaCCttgcatttttgagtgc
ttctgagttgaaaattaaaaccatgaaatttgaactgcgttttattttt
cacc
(SEQ ID NO: 173)

### Sm Binding Domains

The snRNA systems disclosed herein utilize an Sm binding domain (SmBD). The Sm protein ring that assembles around the Sm binding (SmBD) domain to form an snRNP includes SmB/B′, SmD1, SmD2, SmD3, SmE, SmF, SmG. The U7 Sm binding site recruits endogenous RNA binding factors and can be replaced with a non-U7 snRNA to make the snRNA more stable. In one embodiment, the SmBD is selected from the group consisting of U1, U2, U4, and U5 snRNAs. In another embodiment, the SmBD is derived from a pseudo snRNA. In another embodiment, the SmBD is a nucleotide sequence comprising SEQ ID NO: 49 (aATTTTTGGAGca). In another embodiment, the SmBD is a nucleotide sequence comprising SEQ ID NO: 50 (ATTTTT). In another embodiment, the SmBD comprises a nucleotide sequence selected from the group consisting of SEQ ID NO: 51 (AATTTTTGG), SEQ ID NO: 52 (AATTTGTGG), SEQ ID NO: 53 (AATTTGTGG), SEQ ID NO: 54 (AATTTCTGG), SEQ ID NO: 55 (GATTTTTGG), SEQ ID NO: 56 (AATTTTTGA), and SEQ ID NO: 57 (AATTTTTTG). In another embodiment, the SmBD is a nucleotide sequence comprising SEQ ID NO: 92 (AATTTTTGGAGTA).

snRNA Promoters

Gene therapy and RNA-targeting snRNA gene therapy compositions of the disclosure comprise promoter sequences derived from an snRNA. A “promoter” is a regulatory sequence that is a region of a polynucleotide sequence at which initiation and rate of transcription are controlled. It may contain genetic elements at which regulatory proteins and molecules may bind such as RNA polymerase and other transcription factors.

The snRNA systems disclosed herein are operably linked to an snRNA promoter from any of U1-U12. In one embodiment, the snRNA promoter is a U7 promoter. In another embodiment, the U7 promoter is a human U7 promoter (hU7) or a mouse U7 promoter (mU7). In another embodiment, the U7 promoter is an endogenous human U7 promoter at least 65%, 70%, 75%, 80%, 85%, 90%, 95%, 96%, 97%, 98%, 99% or 100% (or any percentage in between) identical to SEQ ID NO: 58: TACTGCCGAATCCAGGTCTCCGGGCTTAACAACAACGAAGGGGCTGTGACTGGC TGCTTTCTCAACCAATCAGCACCGAACTCATTTGCATGGGCTGAGAACAAATGTT CGCGAACTCTAGAAATGAATGACTTAAGTAAGTTCCTTAGAATATTATTTTTCCT ACTGAAAGTTACCACATGCGTCGTTGTTTATACAGTAATAGGAACAAGAAAAAA GTCACCTAAGCTCACCCTCATCAATTGTGGAGTTCCTTTATATCCCATCTTCTCTC CAAACACATACGCA. In one embodiment, the snRNA promoter is a U1 promoter. In another embodiment, the U1 promoter is a human U1 promoter or a mouse U1 promoter.

In other embodiments, the snRNA promoter is a PolII promoter or a PolIII promoter. In other embodiments, the snRNA promoter comprises, consists essentially of, or consists of a nucleic acid sequence at least 65%, 70%, 75%, 80%, 85%, 90%, 95%, 96%, 97%, 98%, 99% or 100% (or any percentage in between) identical to a promoter and/or promoter sequence listed in the Exemplary Promoter Table which follows:

| Exemplary | |
|---|---|
| Promoter | Promoter Sequence |
| hU1 | AAGGACCAGCTTCTTTGGGAGAGAACAGACGCAGGGGGGG |
| | GAGGGAAAAAGGGAGAGGCAGACGTCACTTCCCCTTGGCG |
| | GCTCTGGCAGCAGATTGGTCGGTTGAGTGGCAGAAAGGCA |
| | GACGGGGACTGGGCAAGGCACTGTCGGTGACATCACGGAC |
| | AGGGCGACTTCTATGTAGATGAGGCAGCGCAGAGGCTGAC |
| | GTCTTCGCCACTTGCTGCTTCACCACGAAGGAGTTCCCGT |
| | GCCCTGGGAGCGGGTTCAGGACCGCTGATCGGAAGTGAGA |
| | ATCCCAGCTGTGTGTCAGGGCTGGAAAGGGCTCGGGAGTG |
| | CGCGGGGCAAGTGACCGTGTGTGTAAAGAGTGAGGCGTAT |
| | GAGGCTGTGTCGGGGCAGAGGCacaacgtttc |
| | (SEQ ID NO: 59) |
| hU2 | CCACGCCCTCTGTGAAAGGGCGGGGCATGCAAATTCGAAA |
| | TGAAAGCCCGGGAACGCCGGAAGAAGCACGGGTGTAAGAT |
| | TTCCCTTTTCAAAGGCAGAGAATAAGAAATCAGCCCGAGA |
| | GTGTAAGGGCGTCAATAGCGCTGTGGACGAGACAGAGGGA |
| | ATGGGGCAAGGAGCGAGGCTGGGGCTCTCACCGCGACTTG |
| | AATGTGGATGAGAGTGGGACGGTGACGGCGGGCGCGAAGG |
| | CGAGCGC |
| | (SEQ ID NO: 60) |
| hU4 | TTCGCAGTCTCTGAATTAAGTCTATTAGCATGTTCCTCCC |
| | ATAGTGCTTTGCTTCATATCAACAAAAACCTAGCTAAGTG |
| | AAATCAGCAACGATATGCAGAAACCACCTACGCAGGTCAC |
| | AAACATCTTTCTATGATTGTATAATTTTCAAGCAAGCAAT |
| | AAGTGAAGATTTTTCCATAGGCCCTAAACTCACCTTTGCG |
| | AAATAGGAAGCTGGTTTATTGGGAGTGATGAGCAGGGGGC |
| | GTAACAAATT |
| | (SEQ ID NO: 61) |
| hU5 | GCAGCAAGGCCTCCACTTCACCCCCTAAAGGTTGCCCCAA |
| | GAGCACCGTGTGACTGCTAAGGTATTTCCGGAGTCTAAAG |
| | ACGATTATTCAGGTCTCATTTGCATACCCATAATACACTG |
| | CAAACAGTATTTTTTTCGGAAAAACATTTATATATTGCTT |
| | GACATTTTTAAGTATGAGAATTTTGCATGCAGAATTTTTT |
| | TGTATAAACTTTCTCAGGTAGTAACCCTTGGGATTAGTAG |
| | ACACCATCAGTGTACTAGGAATTGCAGTTACCCGAAAATT |
| | GAGTTACAGAAGTAACTGGT |
| | (SEQ ID NO: 62) |
| hU6 | Gtttattacagggacagcagagatccagtttggttaatta |
| | aggtaccgagggcctatttcccatgattccttcatatttg |
| | catatacgatacaaggctgttagagagataattagaatta |
| | atttgactgtaaacacaaagatattagtacaaaatacgtg |
| | acgtagaaagtaataatttcttgggtagtttgcagtttta |
| | aaattatgttttaaaatggactatcatatgcttaccgtaa |
| | cttgaaagtatttcgatttcttggctttatatatcttgtg |
| | gaaaggacgaaacacc |
| | (SEQ ID NO: 63) |
| h7sk | CTGCAGTATTTAGCATGCCCCACCCATCTGCAAGGCATTC |
| | TGGATAGTGTCAAAACAGCCGGAAATCAAGTCCGTTTATC |
| | TCAAACTTTAGCATTTTGGGAATAAATGATATTTGCTATG |
| | CTGGTTAAATTAGATTTTAGTTAAATTTCCTGCTGAAGCT |
| | CTAGTACGATAAGTAACTTGACCTAAGTGTAAAGTTGAGA |
| | TTTCCTTCAGGTTTATATAGCTTGTGCGCCGCCTGGGTAC |
| | CTC |
| | (SEQ ID NO: 64) |
| tRNA(Val) | CAGGACTAGTCTTTTAGGTCAAAAAGAAGAAGCTTTGTAA |
| | CCGTTGGTTTCCGTAGTGTAGTGGTTATCACGTTCGCCTA |
| | ACACGCGAAAGGTCCCCGGTTCGAAG |
| | (SEQ ID NO: 65) |
| mU1 | TTGTTCCTCTTAGTGTTAATTCACACTAAAGACTGTGCAT |
| | CCGACTCCTACATTTATGAAAGTAAATGCCTGTTGTTAGA |
| | ACAAAAAAGGCTACAGAACAAAAAACAAAGCGAAATACCA |
| | TCTGCTTTAGGTTCAGTGTGGTATTTTCCCGCTGACAGGG |
| | AGGCGGGTTTTTGGGTACAGGAAACGAGTCACTATGGAGG |
| | CGGTACTATGTAGATGAGAATTCAGGTGCAAACTGGGAAA |
| | AGCAACTGCTTCCAAATATTTGTGATTTTTACAGTGTAGT |
| | TTTGGAAAAACTCTTAGCCTACCAATTCTTCTAAGTGTTT |
| | TAAAATGTGGGAGCCAGTACACATGAAGTTATAGAGTGTT |
| | TTAATGAGGCTTAAATATTTACCGTAACTATGAAATGCTA |
| | CGCATATCATGCTGTTCAGGCTCCGTGGCCACGCAACTC |
| | (SEQ ID NO: 66) |
| mU7 | Taacaacataggagctgtgattggctgttttcagccaatc |
| | agcactgActcatttgcatagcctttacaagcggtcacaa |
| | actcaagaaacgagcggttttaatagtcttttagaatatt |
| | gtttatcgaaccgaataaggaactgtgctttgtgattcac |
| | atatcagtggaggggtgtggaaatggcaccttgatctcac |
| | cctcatcgaaagtggagttgatgtcctTccctggctcgct |
| | acagacgcacttccgcaa |
| | (SEQ ID NO: 67) |
| mU7 | taacaacataggagctgtgattggctgttttcagccaatc |
| | agcactgActcatttgcatagcctttacaagcggtcacaa |
| | actcaagaaacgagcggttttaatagtcttttagaatatt |
| | gtttatcgaaccgaataaggaactgtgctttgtgattcac |
| | atatcagtggaggggtgtggaaatggcaccttgatctcac |
| | cctcatcgaaagtggagttgatgtcctTccctggctcgct |
| | acagacgcacttccgc |
| | (SEQ ID NO: 91) |
| hU7 | TACTGCCGAATCCAGGTCTCCGGGCTTAACAACAACGAAG |
| | GGGCTGTGACTGGCTGCTTTCTCAACCAATCAGCACCGAA |
| | CTCATTTGCATGGGCTGAGAACAAATGTTCGCGAACTCTA |
| | GAAATGAATGACTTAAGTAAGTTCCTTAGAATATTATTTT |
| | TCCTACTGAAAGTTACCACATGCGTCGTTGTTTATACAGT |
| | AATAGGAACAAGAAAAAAGTCACCTAAGCTCACCCTCATC |
| | AATTGTGGAGTTCCTTTATATCCCATCTTCTCTCCAAACA |
| | CATACGCA |
| | (SEQ ID NO: 58) |
| mU5 | TGCCAACAGCTTTGAAATCCTCTGTGCTCTTGTGCCAATC |
| | CCACCCTCAGAACAGGGAGTCTGTCGGTTATGTTTTCGTC |
| | CTTTACAGTTCTTCGCCGTCTAATCCATTGGAAACCTCCT |
| | TTATTTAAAGGAAACCGTGTCCAAATTCAGTGTACAGAGG |
| | TGCAAACCTCCGCCCCGCTCCTTGTAGGAAGCCCACGGGA |
| | ACTGGGCCAATCGGACGGCGCTTACTCGGCTCATTTACAT |
| | ACCCATAACACACCGCGGCTAATGCAAATATTTTCGTGAA |
| | AAGATCTTCCATTACTCGAAGTTTTAAAACTAAAAACAAA |
| | ATATTATTTCAAAAGATCATGAAGAAATTTCTGTCGGTAA |
| | CAGCAGTTTCAATTGATAAATCACCATCACTTATCTAGGA |
| | GGTTCTGTTACTCTAGAAGTGAATTAAGCAGGACAGCTGT |
| | (SEQ ID NO: 68) |
| mU2 | Gggcggggcatgcaaataactgctctgtggaactctggga |
| | gcaaaaacaaaaaactgcaaccaaaacttctcggcctcct |
| | tgaatcttacaggcttttcgtggcgtaaaggtggtgtact |
| | caatgaagaggagagtctgtgttggctgcatgtttgagtc |
| | ggttggttggtgactgtgaatTAAAGGTGTGGtcggtgtt |
| | gagtgtatggggcgtgtgggcglagttcggt |
| | (SEQ ID NO: 69) |
| mU6 | cgacgccgccatctctaggcccgcgccggccccctcgcac |
| | agacttgtgggagaagctcggctactcccctgccccggtt |
| | aatttgcatataatatttcctagtaactatagaggcttaa |
| | tgtgcgataaaagacagataatctgttctttttaatacta |
| | gctacattttacatgataggcttggatttctataagagat |
| | acaaatactaaattattattttaaaaaacagcacaaaagg |
| | aaactcaccctaactgtaaagtaattgtgtgttttgagac |
| | tataaatatcccttggagaaaagccttgttt |
| | (SEQ ID NO: 70) |
| mH1 | CATgCAAATTACGCGCTgTGCTTTGTGGGAAATCACCCTA |
| | AACGTAAAATTTATTCCTCTTTcGAGCCTTATAGTGGcGG |
| | CCGGTCTACACCCTAAA |
| | (SEQ ID NO: 71) |

### Terminator Sequences

The snRNA systems disclosed herein comprise an snRNA downstream terminator (DT). In one embodiment the snRNA DT is a U7 DT comprising, consisting essentially of, or consisting of a nucleic acid sequence at least 65%, 70%, 75%, 80%, 85%, 90%, 95%, 96%, 97%, 98%, 99% or 100% (or any percentage in between) identical to SEQ ID NO: 72: CCTCTTATGATGTTTGTTGCCAATGATAGATTGTTTTCACTGTGCAAAAATTATGG GTAGTTTTGGTGGTCTTGATGCAGTTGTAAGCTTGGAG.

In another embodiment, the snRNA systems disclosed herein comprise any promoter selected from the exemplary promoter table above and any DT selected from the exemplary DT table below. Such promoters and DTs do not need to correspond (e.g., U1 promoter, U1 DT) to each other (e.g., U7 promoter, U1 DT).

In another embodiment, the DT comprises, consists essentially of, or consists of a nucleic acid sequence at least 65%, 70%, 75%, 80%, 85%, 90%, 95%, 96%, 97%, 98%, 99% or 100% (or any percentage in between) identical to the exemplary DTs and/or DT sequences listed in the Exemplary DT Table below:

| Exemplary DT | DT Sequence |
|---|---|
| hU1 | ACTTTCTGGAGTTTCAAAAACAGACT |
| | GTACGCCAAGGGTCATATCTTTTT |
| | SEQ ID NO: 73 |
| hU2 | CCGGGGATACAACGTGTTTCCTAAAA |
| | GTAGAGGGAGGTAAGAGACGGTAG |
| | SEQ ID NO: 74 |
| hU4 | CTGAATTTTCTTGCAGTTGAACAACA |
| | GAGGCTTTTTTTGTGTGTGTGGGG |
| | SEQ ID NO: 75 |
| hU5 | ATATGTGGTAATCCAACAATAGAAAT |
| | TATTTTTAAGTTTGTGTGTTCCTT SEQ |
| | ID NO: 76 |
| hU6 | TTTTTT |
| | SEQ ID NO: 77 |
| h7sk | TTTTTT |
| | SEQ ID NO: 78 |
| tRNA(Val) | TTTTTT |
| | SEQ ID NO: 79 |
| mU1 | GTTTACTTGGTTTTAAAAATAGCTTG |
| | CACTAGCGATACGGAATATGGTTATT |
| | AGGTTTGTTAGGCATCATGTCGTGTC |
| | TTACTATAGAAAAATAACGTAGTGTT |
| | CATTTTAGCCTGCCTGTATGTGTTAA |
| | TTTGTCCTTATTGCGCATTGTTCTTGT |
| | TAAGTCTTCTGTAAGGAGTTGCGGGT |
| | TTCAAACTGTCAGTCTGAGAGCA |
| | SEQ ID NO: 80 |
| mU7 | Cccaatttcactggtctacaatgaaagca |
| | aaacagttctcttccccgctccccggtgt |
| | gtgagaggggctttgatccttctctggt |
| | ttcctaggaaacgcgtatgtg |
| | SEQ ID NO: 81 |
| hU7 | CCTCTTATGATGTTTGTTGCCAATGA |
| | TAGATTGTTTTCACTGTGCAAAAATT |
| | ATGGGTAGTTTTGGTGGTCTTGATGC |
| | AGTTGTAAGCTTGGAG |
| | SEQ ID NO: 72 |
| mU7+ | Cccaatttcactggtctacaatgaaagca |
| | aaacagttctcttccccgctccccggtgt |
| | gtgagaggggctttgatccttctctggt |
| | ttcctaggaaacgcgtatgtgtac |
| | SEQ ID NO: 82 |
| hU7 | CTTATGATGTTTGTTGCCAATGATAG |
| | ATTGTTTTCACTGTGCAAAAATTATG |
| | GGTAGTTTTGGTGGTCTTGATGCAGT |
| | TGTAAGCTTGGAG SEQ ID NO: 83 |
| mU5 | CCACATTTTGTGTTTAAAAATAGAAA |
| | TATTTAAGTGAGATCAGTTTAAATCT |
| | GCTTTATCTAGGGTGTCTAACTGCTT |
| | GCATCTTTTTAACTTTTCCTTATTTGT |
| | GAGAAGGTCTGTAAAACTTATTATAT |
| | GTTAGTACACTGTAGCTGTCTTCAGA |
| | CACTCCAGAAGAGGGATTCAGATCA |
| | CCTTACAGATGGTTTTGA |
| | SEQ ID NO: 84 |
| mU2 | CCCTCTGGGGAgtaaagttggttttaa |
| | agtcagagcatggtgattgtagggcag |
| | tccaacttttttaaatat |
| | gctgtg SEQ ID NO: 85 |
| mU6V | TTTTTT SEQ ID NO: 86 |
| mH1 | TTTTTT SEQ ID NO: 87 |

### AAV Vectors

In one embodiment, the snRNA is delivered in an AAV vector.

In some embodiments, the AAV vector comprises multiple copies of the snRNA. In some embodiments, the multiple copies of the snRNA are 2, 3, or 4 copies of the snRNA. In some embodiments, the multiple copies of the snRNA are 4 or more copies of the snRNA. In some embodiments, the AAV vector comprises multiple snRNA where each sRNA targets a different RNA sequence.

### Buffer Sequences

In some embodiments, each snRNA of the multiple copies of snRNA is separated by a nucleic acid buffer sequence derived from human non-coding genomic sequences downstream of an snRNA. In one embodiment, the buffer sequence is derived from human genomic sequences downstream of U7.

In one embodiment, the buffer sequence comprises, consists essentially of, or consists of a nucleic acid sequence at least 65%, 70%, 75%, 80%, 85%, 90%, 95%, 96%, 97%, 98%, 99% or 100% (or any percentage in between) identical to the group consisting of the following nucleic acid sequences:

buffer 1 (30 bp)
(SEQ ID NO: 42)
CAAACTACAGAGCCAAGTGCTATCCACAGA,
buffer 2 (30 bp)
(SEQ ID NO: 43)
GAGCTTTCTGGGTTGCCATCTCAAGCAGAC,
buffer 3 (30 bp)
(SEQ ID NO: 44)
TACAAGGCCATCAGCTCATACTCACAATTG,

and a combination thereof.

In another embodiment, the buffer sequence comprises, consists essentially of, or consists of a nucleic acid sequence at least 65%, 70%, 75%, 80%, 85%, 90%, 95%, 96%, 97%, 98%, 99% or 100% (or any percentage in between) identical to the group consisting of the following nucleic acid sequences:

buffer 1 (100 bp)
(SEQ ID NO: 45)
CAAACTACAGAGCCAAGTGCTATCCACAGAGAGCTTTCTGG
GTTGCCATCTCAAGCAGACTACAAGGCCATCAGCTCATACT
CACAATTGACTTTGAGAG,
buffer 2 (100 bp)
(SEQ ID NO: 46)
TTGACCACATACGTGCTCTTTCAAAGTTCTGTGTTTGAAGTT
ATGTTAGTAACAACTGATGCCCATCCTGCAATGACAAATCCA
ATTCTCAGTGCAGCTC,

and a combination thereof.

In another embodiment, the buffer sequence comprises, consists essentially of, or consists of a nucleic acid sequence at least 65%, 70%, 75%, 80%, 85%, 90%, 95%, 96%, 97%, 98%, 99% or 100% (or any percentage in between) identical to the group consisting of the following nucleic acid sequences:

buffer 1 (500 bp)
(SEQ ID NO: 47)
CAAACTACAGAGCCAAGTGCTATCCACAGAGAGCTTTCTGGGTTG
CCATCTCAAGCAGACTACAAGGCCATCAGCTCATACTCACAATTG
ACTTTGAGAGTCATTTTCCAATGCTCCTACACACCCCTTCTTCAC
AATCCCCAACAAATCTGAGGCTGGAACTTGGTACCATAACAATCA
TTACATTATTTCACCAGAAGTACACCTTGCCTGGAAGATTGGCAT
TATAGCATCTTCTAACATTGTGAAAGTTAGTGACCAATGAGGAGA
TCCAAGTCAGTTCCAGTTGGATTTCTCTATACTCTATAATAAATA
TATATGGTGTCTTCAACAATAGGACTTTGCCATCCAGTGATGCTA
AAAATCAATAACAATGGCAATAACCTGCCCTGTTTGGAAAGCCTC
TGGCTTCCATGACTAACAATTCAAGGCAGGTCTCCTATACCTAGT
ACTGAGATTTTTATTTGATAAACTATATCTTCTGGGAGGAGAAGC
ATTGT,
buffer 2 (500 bp)
(SEQ ID NO: 48)
TTGACCACATACGTGCTCTTTCAAAGTTCTGTGTTTGAAGTTATG
TTAGTAACAACTGATGCCCATCCTGCAATGACAAATCCAATTCTC
AGTGCAGCTCTCTGAAATAGTTTTGCTTTCTCTCTCTAGGTCTGT
TCTATACTCCTAACTCTCCAGGAGTTTACAAGGAATAAAATCTCT
TCCAAATGCTTTCTGTTGCAACAACTGGACCATACTGAAAGCTGA
GGCCCACAATTGCAATCTAGGTTAGCAGGTAATCATTGTTGGTGA
GGTCCTCCCTTTCCCCAGGCTCGTGTTTGTATTGGGGAGCAGGAA
ATTTTTGCTAGAGCAGCACTGCCATCTCTCTACACTCCACCTGAT
TGGTGGGATGGACCAGAGAAATGGACATTCCCAACACAGTCCCTC
CTTTCACATCTGCTCACCTGCCCACAGGATACTTTCCACCATGCA
TACTGGGCTCTGCACCAACCATTCAGCAGTGATGAAGAGGAAACT
TGAAC,

and/or a combination thereof.

The 100 bp and 500 bp buffer 1 sequences are derived from a sequence starting 100 bp downstream of the Mus musculus U7 pseudogene 8 (Location Chromosome 14: 4,409,359-4,409,421 reverse strand. GRCm39:CM001007.3). The 100 bp and 500 bp buffer 2s are derived from the sequence starting 130 bp downstream of human U7 pseudogene 5 (Chromosome X: 140,451, 148-140,451,208 forward strand. GRCh38:CM000685.2). Both 100 bp buffers are the first 100 bp of the corresponding 500 bp buffer. The 30 bp buffers 1, 2, and 3, are sequential 30 bp sequences within “100 bp buffer 1”, downstream of the Mus musculus U7 pseudogene 8. These downstream sequences were selected due to the lack of any known regulatory sites or genes within or nearby to the sequence (using Gencode/Ensembl), in addition to lack of repetitive sequence, 40-60% GC content for total buffer, 40-60% GC content in the 20 bp region at both ends of the buffer, and minimal sequence complexity.

Exemplary snRNA constructs are as follows:

SOD1 (Superoxide dismutase) contains a toxic mutation which leads to SOD1 proteins responsible for ALS (Amyotrophic lateral sclerosis). Using the disclosed editing snRNA provided herewith, the SOD1 start codon can be targeted and edited leading to a decrease in SOD1 expression.

Exemplary SOD1 editing constructs are as follows:

A05627: pcDNA3.1_mU1_SOD1_AUG_Edit_70 nt no mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | CCGTCGCCCTTCAGCACGCACACGGCCTTCGTCGCCATAACTCGCTAGGCC |
| seq. | ACGCCGAGGTCCTGGTTCC (SEQ ID NO: 124) |
| smOPT | aATTTTTGGAGca (SEQ ID NO: 49) |
| ms-short-eSL | Ggttttctgacctccgtoggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05628: pcDNA3.1_mU1_SOD1_AUG_Edit_70 nt + mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | CCGTCGCCCTTCAGCACGCACACGGCCTTCGTCGCCAcAACTCGCTAGGCC |
| seq. | ACGCCGAGGTCCTGGTTCC (SEQ ID NO: 125) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05629: pcDNA3.1_mU1_SOD1_AUG_Edit_111 nt no mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | ATGATGCCCTGCACTGGGCCGTCGCCCTTCAGCACGCACACGGCCTTCGTC |
| seq. | GCCATAACTCGCTAGGCCACGCCGAGGTCCTGGTTCCGAGGACTGCAACG |
| | GAAACCCCAG (SEQ ID NO: 126) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05630: pcDNA3.1_mU1_SOD1_AUG_Edit_111 nt + mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | ATGATGCCCTGCACTGGGCCGTCGCCCTTCAGCACGCACACGGCCTTCGTC |
| seq. | GCCAcAACTCGCTAGGCCACGCCGAGGTCCTGGTTCCGAGGACTGCAACG |
| | GAAACCCCAG (SEQ ID NO: 127) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05631: pcDNA3.1_mU1_SOD1_AUG_Edit_151 nt no mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | TCCTTCTGCTCGAAATTGATGATGCCCTGCACTGGGCCGTCGCCCTTCAGC |
| seq. | ACGCACACGGCCTTCGTCGCCATAACTCGCTAGGCCACGCCGAGGTCCTG |
| | GTTCCGAGGACTGCAACGGAAACCCCAGACGCTGCAGGAGACTACGACG |
| | C (SEQ ID NO: 128) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05632: pcDNA3.1_mU1_SOD1_AUG_Edit_151 nt + mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | TCCTTCTGCTCGAAATTGATGATGCCCTGCACTGGGCCGTCGCCCTTCAGC |
| seq. | ACGCACACGGCCTTCGTCGCCAcAACTCGCTAGGCCACGCCGAGGTCCTG |
| | GTTCCGAGGACTGCAACGGAAACCCCAGACGCTGCAGGAGACTACGACG |
| | C (SEQ ID NO: 129) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05633: pcDNA3.1_mU1_SOD1_AUG_Edit + ADAR RD_20 nt 3, 2, 1
no mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | GTCGCCATAACTCGCTAGGCaaaCGCCGAGGTCCTGGTaaaGCAACGGAAAC |
| seq | CCC AaaaGCAGGAGACTACGAC (SEQ ID NO: 130) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05634: pcDNA3.1_mU1_SOD1_AUG_Edit + ADAR RD_20 nt 3, 2, 1 (+) mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | GTCGCCAcAACTCGCTAGGCaaaCGCCGAGGTCCTGGTaaaGCAACGGAAAC |
| seq | CCC AaaaGCAGGAGACTACGAC (SEQ ID NO: 130) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05635: pcDNA3.1_mU1_SOD1_AUG Edit_+ADAR RD_5, 4, 20 nt, 2 no mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | GCCCTGCACTGGGCCaaaAGCACGCACACGGCCaaaGTCGCCATAACTCGCT |
| seq | AGGCaaaGCAACGGAAACCCCA (SEQ ID NO: 131) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05636: pcDNA3.1_mU1_SOD1_AUG Edit_+ADAR RD_5, 4, 20 nt, 2 (+)mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | GCCCTGCACTGGGCCaaaAGCACGCACACGGCCaaaGTCGCCAcAACTCGCT |
| seq | AGGCaaaGCAACGGAAACCCCA (SEQ ID NO: 132) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05637: pcDNA3.1_mU1 SOD1_AUG Edit_+ADAR RD_70 nt no mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | GTCGCCCTTCAGCACGCACACGGCCTTCGTCGCCATAACTCGCTAGGCCAC |
| seq | GCCGAGGTCCTGGTTCCGAG (SEQ ID NO: 133) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05638: pcDNA3.1_mU1 SOD1_AUG_Edit_+ADAR RD_70 nt (+)mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | GTCGCCCTTCAGCACGCACACGGCCTTCGTCGCCAcAACTCGCTAGGCCAC |
| seq | GCCGAGGTCCTGGTTCCGAG (SEQ ID NO: 134) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05639: pcDNA3.1_aRmU7_ISD_nt_105 bp

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Non- | tgaacagctcctcgcccttgctcactggcagagccctcCagcatcgcga |
| targeting- | gcaggcgctgcctcctccgcc (SEQ ID NO: 151) |
| seq. | |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05631: pcDNA3.1_mU1 SOD1_AUG_Edit_+ADAR RD_111 nt (+)mismatch

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | ATGATGCCCTGCACTGGGCCGTCGCCCTTCAGCACGCACACGGCCTTCGTC |
| seq. | GCCAcAACTCGCTAGGCCACGCCGAGGTCCTGGTTCCGAGGACTGCAACG |
| | GAAACCCCAG (SEQ ID NO: 127) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05632: pcDNA3.1_mU1 SOD1_AUG_Edit_+ADAR RD_151 nt (+)mismatch.3

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | TCCTTCTGCTCGAAATTGATGATGCCCTGCACTGGGCCGTCGCCCTTCAGC |
| seq. | ACGCACACGGCCTTCGTCGCCAcAACTCGCTAGGCCACGCCGAGGTCCTG |
| | GTTCCGAGGACTGCAACGGAAACCCCAGACGCTGCAGGAGACTACGACG |
| | C (SEQ ID NO: 129) |
| smOPT | aATTTTTGGAGca (SEQ ID NO: 49) |
| ms-short-eSL | Ggttttctgacctccgtoggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05933: pcDNA3.1_mU1_SOD1_AUG_Edit_+ADAR RD_40 nt 5, 4, sd, 2 (+) mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | GCCCTGCACTGGGCCaaaAGCACGCACACGGCCaaaGTCGCCAcAACTCGCT |
| seq. | AGGCCACGCCGAGGTCCTGGTTCCaaaGCAACGGAAACCCCA (SEQ ID NO: |
| | 135) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05934: pcDNA3.1_mU1_SOD1_AUG_Edit_+ADAR RD_46 nt (+) mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | CGTCGCCACAACTCGCTAGGCCACGCCGAGGTCCTGGTTCCGAGGAC (SEQ |
| seq. | ID NO: 136) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05935: pcDNA3.1_mU1_SOD1_AUG_Edit_+ADAR RD_70 nt + 10 nt (+) mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | GCACTGGGCCGTCGCCCTTCAGCACGCACACGGCCTTCGTCGCCAcAACTC |
| seq. | GCTAGGCCACGCCGAGGTCC (SEQ ID NO: 137) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05936: pcDNA3.1_mU1_SOD1_AUG_Edit_+ADAR RD_70 nt + 20 nt (+) mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | ATGATGCCCTGCACTGGGCCGTCGCCCTTCAGCACGCACACGGCCTTCGTC |
| seq. | GCCAcAACTCGCTAGGCCAC (SEQ ID NO: 138) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05937: pcDNA3.1_mU1 SOD1_AUG_Edit_+ADAR RD_70 nt + 30 (+)mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | CTCGAAATTGATGATGCCCTGCACTGGGCCGTCGCCCTTCAGCACGCACA |
| seq. | CGGCCTTCGTCGCCAcAACTC (SEQ ID NO: 139) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05938: pcDNA3.1_mU1 SOD1_AUG_Edit_+ADAR RD_111 nt + 10 nt (+)mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | CTCGAAATTGATGATGCCCTGCACTGGGCCGTCGCCCTTCAGCACGCACA |
| seq. | CGGCCTTCGTCGCCAcAACTCGCTAGGCCACGCCGAGGTCCTGGTTCCGAG |
| | GACTGCAACG (SEQ ID NO: 139) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05939: pcDNA3.1_mU1 SOD1_AUG_Edit_+ADAR RD_111 nt + 20 (+)mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | TTTCCTTCTGCTCGAAATTGATGATGCCCTGCACTGGGCCGTCGCCCTTCA |
| seq. | GCACGCACACGGCCTTCGTCGCCAcAACTCGCTAGGCCACGCCGAGGTCCT |
| | GGTTCCGAG (SEQ ID NO: 140) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05940: pcDNA3.1_mU1 SOD1_AUG_Edit_+ADAR RD_111 nt + 30 (+)mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | GGTCCATTACTTTCCTTCTGCTCGAAATTGATGATGCCCTGCACTGGGCCG |
| seq. | TCGCCCTTCAGCACGCACACGGCCTTCGTCGCCAcAACTCGCTAGGCCACG |
| | CCGAGGTCC (SEQ ID NO: 141) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05941: pcDNA3.1_mU1 SOD1_AUG_Edit_+ADAR RD_111 nt + 40 (+)mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | CACCTTCACTGGTCCATTACTTTCCTTCTGCTCGAAATTGATGATGCCCTGC |
| seq. | ACTGGGCCGTCGCCCTTCAGCACGCACACGGCCTTCGTCGCCAcAACTCGC |
| | TAGGCCAC (SEQ ID NO: 142) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05942: pcDNA3.1_mU1 SOD1_AUG_Edit_+ADAR RD_150 nt + 10 nt (+)mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | TCCATTACTTTCCTTCTGCTCGAAATTGATGATGCCCTGCACTGGGCCGTC |
| seq. | GCCCTTCAGCACGCACACGGCCTTCGTCGCCAcAACTCGCTAGGCCACGCC |
| | GAGGTCCTGGTTCCGAGGACTGCAACGGAAACCCCAGACGCTGCAGGAG |
| | (SEQ ID NO: 143) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05943: pcDNA3.1_mU1 SOD1_AUG_Edit_+ADAR RD_150 nt + 20 (+)mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | CCTTCACTGGTCCATTACTTTCCTTCTGCTCGAAATTGATGATGCCCTGCAC |
| seq. | TGGGCCGTCGCCCTTCAGCACGCACACGGCCTTCGTCGCCAcAACTCGCTA |
| | GGCCACGCCGAGGTCCTGGTTCCGAGGACTGCAACGGAAACCCCAGAC |
| | (SEQ ID NO: 144) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05944: pcDNA3.1_mU1_SOD1_AUG_Edit_+ADAR RD_150 nt + 30 (+)mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | CTTCCCCACACCTTCACTGGTCCATTACTTTCCTTCTGCTCGAAATTGATGA |
| seq. | TGCCCTGCACTGGGCCGTCGCCCTTCAGCACGCACACGGCCTTCGTCGCCA |
| | cAACTCGCTAGGCCACGCCGAGGTCCTGGTTCCGAGGACTGCAACGGA |
| | (SEQ ID NO: 145) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05945: pcDNA3.1_mU1_SOD1_AUG_Edit_+ADAR RD_150 nt + 40 (+)mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | TCCTCGGAACCAGGACCTCGGCGTGGCCTAGCGAGTTgTGGCGACGAAGG |
| seq. | CCGTGTGCGTGCTGAAGGGCGACGGCCCAGTGCAGGGCATCATCAATTTC |
| | GAGCAGAAGGAAAGTAATGGACCAGTGAAGGTGTGGGGAAGCATTAAAG |
| | GA (SEQ ID NO: 146) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05946: pcDNA3.1_mU1 SOD1_AUG_Edit_+ADAR RD_150 nt + 50 (+)mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | CTTCAGTCAGTCCTTTAATGCTTCCCCACACCTTCACTGGTCCATTACTTTC |
| seq. | CTTCTGCTCGAAATTGATGATGCCCTGCACTGGGCCGTCGCCCTTCAGCAC |
| | GCACACGGCCTTCGTCGCCAcAACTCGCTAGGCCACGCCGAGGTCCTG |
| | (SEQ ID NO: 147) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05947: pcDNA3.1_mU1_SOD1_AUG_Edit_+ADAR RD_20 nt 5, 4, SD,
6 enhanced size (+) mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | GCCCTGCACTGGGCCGaaaCAGCACGCACACGGCCaaaGTCGCCAcAACTCG |
| seq. | CTAGGCaaaGCAACGGAAACCCCAGACGC (SEQ ID NO: 148) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05948: pcDNA3.1_mU1_SOD1_AUG_Edit_+ADAR RD_40 nt 5, 4, SD,
6 enhanced size (+) mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

A05949: pcDNA3.1_mU1_SOD1_AUG_Edit_+ADAR RD_45 nt 5, 4, SD, 6
enhanced size (+) mismatch.

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | GCCCTGCACTGGGCCGaaaCAGCACGCACACGGCCaaaGTCGCCAcAACTCG |
| seq. | CTAGGCCACGCCGAGGTCCTGGTTCCGAGGACaaaGCAACGGAAACCCCAG |
| | ACGC (SEQ ID NO: 150) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

IDUA (alpha-L-iduronidase (IDUA): Most MPS (Mucopolysaccharidosis) I-H patients have a premature stop codon mutation in one or both alleles and are unable to synthesize a full-length polypeptide causing a loss of enzyme activity. The two alpha-L-iduronidase (IDUA) gene premature stop codons, Q70X and W402X, are the most common (70%) mutations in MPS I patients, a rare, recessively inherited lysosomal storage disorder, a progressive, multi-systemic disease caused by a reduced or absent IDUA enzyme activity secondary to biallelic loss-of-function variants in the IDUA. snRNA-mediated targeting of IDUA W402X/Exon 9 provides improved stability and less immunogenicity/off-targets compared to prime editors or traditional gene therapy enzyme replacement.

Exemplary IDUA editing constructs are as follows:

A05318: Stuffer-scAAV-2x aR mU7_ISD_IDUA(W402X) mature-mRNA 105 bp

| ITR | Ctgcgcgctcgctcgctcactgaggccgcccgggcaaagcccgg |
|---|---|
| | gcgtcgggcgacctttggtcgcccggcctcagtgagcgagcgag |
| | cgcgcagagagggagtggggtt (SEQ ID NO: 89) |
| U7 promoter | taacaacataggagctgtgattggctgttttcagccaatcagca |
| | ctgActcatttgcatagcctttacaagcggtcacaaactcaaga |
| | aacgagcggttttaatagtcttttagaatattgtttatcgaacc |
| | gaataaggaactgtgctttgtgattcacatatcagtggaggggt |
| | gtggaaatggcaccttgatctcaccctcatcgaaagtggagttg |
| | atgtcctTccctggctcgctacagacgcacttccgc |
| | (SEQ ID NO: 91) |
| 5′ISD | aaggagt |
| Targeting | GCCCACCGTGTGGTTGCTGTCCAGGACGGTCCCGGCCTGCGACACTTCGG |
| Sequence | CCCAGAGCTGCTCCTCATCCAGCAGCGCCAGCAGCCCCATGGCCGTGAGC |
| | ACCGG (SEQ ID NO: 153) |
| smOPT | aATTTTTGGAGca (SEQ ID NO: 49) |
| ms-eSL | Ggttttctgacctccgtcggaaaacccct (SEQ ID NO: 14) |
| U7 | Cccaatttcactggtctacaatgaaagcaaaacagttctcttccccgctc |
| termination | cccggtgtgtgagaggggctttgatccttctctg |
| | gtttcctaggaaacgcgtatgtg (SEQ ID NO: 81) |
| U1 promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTC (SEQ ID NO: 66) |
| 5′ISD | ggagt |
| Targeting | GCCCACCGTGTGGTTGCTGTCCAGGACGGTCCCGGCCTGCGACACTTCGG |
| Sequence | CCCAGAGCTGCTCCTCATCCAGCAGCGCCAGCAGCCCCATGGCCGTGAGC |
| | ACCGG (SEQ ID NO: 153) |
| smOPT | aATTTTTGGAGca (SEQ ID NO: 49) |
| ms-eSL | Ggttttctgacctccgtcggaaaacccct (SEQ ID NO: 14) |
| U1 | GTTTACTTGGTTTTAAAAATAGCTTGCACTAGCGATACGGAATATGGTTAT |
| termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTC |
| | TTGTTAAGTCTTCTGTAAGGAGTTGCGGGTTTCAAACTGTCAGTCTGAGAG |
| | CA (SEQ ID NO: 80) |
| ITR | Aggaacccctagtgatggagttggccactccctctctgegcgctcgctcgct |
| | cactgaggccgggcgaccaaaggtcgcccgacgcccgggctttgcccgggcg |
| | gcctcagtgagcgagcgagcgcgcagctgcctgcagg (SEQ ID NO: |
| | 90) |

In one embodiment the A05318 vector comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 177.

A05319: Stuffer-scAAV-2x_aR mU7_ISD_IDUA(W402X) mature-mRNA 105 bp + ADAR

| | |
|---|---|
| ITR | Ctgcgcgctcgctcgctcactgaggccgcccgggcaaagcccgggcgtcggg |
| | cgacctttggtcgcccggcctcagtgagcgagcgagcgcgcagagagggagt |
| | ggggtt (SEQ ID NO: 89) |
| U7 promoter | taacaacataggagctgtgattggctgttttcagccaatcagcactgActca |
| | tttgcatagcctttacaagcggtcacaaactcaagaaacgagcggttttaata |
| | gtcttttagaatattgtttatcgaaccgaataaggaactgtgctttgtgattc |
| | acatatcagtggaggggtgtggaaatggcaccttgatctcaccctcatcgaaa |
| | gtggagttgatgtcctTccctggctcgctacagacgcacttccgc |
| | (SEQ ID NO: 91) |
| 5′ISD | aaggagt |
| ADAR | GGTGTCGAGAAGAGGAGAACAATATGCTAAATGTTGTTCTCGTCTCCTCG |
| recruiting | ACACC (SEQ ID NO: 88) |
| sequence | |
| Targeting | GCCCACCGTGTGGTTGCTGTCCAGGACGGTCCCGGCCTGCGACACTTCGG |
| Sequence | CCCAGAGCTGCTCCTCATCCAGCAGCGCCAGCAGCCCCATGGCCGTGAGC |
| | ACCGG (SEQ ID NO: 153) |
| smOPT | aATTTTTGGAGca (SEQ ID NO: 49) |
| ms-eSL | Ggttttctgacctccgtcggaaaacccct (SEQ ID NO: 14) |
| U7 | Cccaatttcactggtctacaatgaaagcaaaacagttctcttccccgctccc |
| termination | cggtgtgtgagaggggctttgatccttctctggtttcctaggaaacgcgtat |
| | gtg (SEQ ID NO: 81) |
| U1 promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTC (SEQ ID NO: 66) |
| 5′ISD | ggagt |
| ADAR | GGTGTCGAGAAGAGGAGAACAATATGCTAAATGTTGTTCTCGTCTCCTCG |
| recruiting | ACACC (SEQ ID NO: 88) |
| sequence | |
| Targeting | GCCCACCGTGTGGTTGCTGTCCAGGACGGTCCCGGCCTGCGACACTTCGG |
| Sequence | CCCAGAGCTGCTCCTCATCCAGCAGCGCCAGCAGCCCCATGGCCGTGAGC |
| | ACCGG (SEQ ID NO: 153) |
| smOPT | aATTTTTGGAGca (SEQ ID NO: 49) |
| ms-eSL | Ggttttctgacctccgtcggaaaacccct (SEQ ID NO: 14) |
| U1 | GTTTACTTGGTTTTAAAAATAGCTTGCACTAGCGATACGGAATATGGTTAT |
| termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |
| ITR | Aggaacccctagtgatggagttggccactccctctctgcgcgctcgctcgctcac |
| | tgaggccgggcgaccaaaggtcgcccgacgcccgggctttgcccgggcggcctca |
| | gtgagcgagcgagcgcgcagctgcctgcagg (SEQ ID NO: 90) |

In one embodiment the A05319 vector comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 178.

A05320: Stuffer-scAAV-2x_aR mU7_ISD_IDUA(W402X) mature-mRNA
ADAR_20 bp_3xRS

| | |
|---|---|
| ITR | Ctgcgcgctcgctcgctcactgaggccgcccgggcaaagcccgggcgtcgggcgac |
| | ctttggtcgcccggcctcagtgagcgagcgagcgcgcagagagggagtggggtt |
| | (SEQ ID NO: 89) |
| U7 promoter | taacaacataggagctgtgattggctgttttcagccaatcagcactgActcatttgcat |
| | agcctttacaagcggtcacaaactcaagaaacgagcggttttaatagtcttttagaata |
| | ttgtttatcgaaccgaataaggaactgtgctttgtgattcacatatcagtggaggggtg |
| | tggaaatggcaccttgatctcaccctcatcgaaagtggagttgatgtcctTccctggct |
| | cgctacagacgcacttccgc |
| | (SEQ ID NO: 91) |
| 5′ISD | aaggagt |
| ADAR | GGTGTCGAGAAGAGGAGAACAATATGCTAAATGTTGTTCTCGTCTCCTCG |
| recruiting | ACACC (SEQ ID NO: 88) |
| sequence | |
| Targeting | TTCGGCCCAGAGCTGCTCCTAAAGGCCGTGAGCACCGGAAACGTGCGGCG |
| Sequence | GGCGGGAAAGCGCGAAGGGGTGCGGGTGG (SEQ ID NO: 154) |
| smOPT | aATTTTTGGAGca (SEQ ID NO: 49) |
| ms-eSL | Ggttttctgacctccgtcggaaaacccct (SEQ ID NO: 14) |
| U7 | Cccaatttcactggtctacaatgaaagcaaaacagttctcttccccgctccc |
| termination | cggtgtgtgagaggggctttgatccttctctggtttcctaggaaacgcgtat |
| | gtg (SEQ ID NO: 81) |
| U1 promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTC (SEQ ID NO: 66) |
| 5′ISD | ggagt |
| ADAR | GGTGTCGAGAAGAGGAGAACAATATGCTAAATGTTGTTCTCGTCTCCTCG |
| recruiting | ACACC (SEQ ID NO: 88) |
| sequence | |
| Targeting | TTCGGCCcAGAGCTGCTCCTAAAGGCCGTGAGCACCGGAAACGTGCGGCG |
| Sequence | GGCGGGAAAGCGCGAAGGGGTGCGGGTGG (SEQ ID NO: 154) |
| smOPT | aATTTTTGGAGca (SEQ ID NO: 49) |
| ms-eSL | Ggttttctgacctccgtcggaaaacccct (SEQ ID NO: 14) |
| U1 | GTTTACTTGGTTTTAAAAATAGCTTGCACTAGCGATACGGAATATGGTTAT |
| termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |
| ITR | Aggaacccctagtgatggagttggccactccctctctgcgcgctcgctcgct |
| | cactgaggccgggcgaccaaaggtcgcccgacgcccgggctttgcccggggg |
| | cctcagtgagcgagcgagcgcgcagctgcctgcagg |
| | (SEQ ID NO: 90) |

In one embodiment the A05320 vector comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 179.

A05322: Stuffer-scAAV-2x aR mU7_ISD_IDUA(W402X) Non-Targeting + ADAR

| | |
|---|---|
| ITR | Ctgcgcgctcgctcgctcactgaggccgcccgggcaaagcccgggcgtcgggcgacctt |
| | tggtcgcccggcctcagtgagcgagcgagcgcgcagagagggagtggggtt |
| | (SEQ ID NO: 89) |
| U7 promoter | taacaacataggagctgtgattggctgttttcagccaatcagcactgActcatttgcat |
| | agcctttacaagcggtcacaaactcaagaaacgagcggttttaatagtcttttagaata |
| | ttgtttatcgaaccgaataaggaactgtgctttgtgattcacatatcagtggaggggtg |
| | tggaaatggcaccttgatctcaccctcatcgaaagtggagttgatgtcctTccctggct |
| | cgctacagacgcacttccgc |
| | (SEQ ID NO: 91) |
| 5′ISD | aaggagt |
| ADAR | GGTGTCGAGAAGAGGAGAACAATATGCTAAATGTTGTTCTCGTCTCCTCG |
| recruiting | ACACC (SEQ ID NO: 88) |
| sequence | |
| Non- | Gctacgtcaataggcactaacttattggcgctggtgaacggacttcctctcgagtac |
| Targeting | cagaagatgactacaaaactcctttccattgcgagtatcggagtctgg |
| Sequence | (SEQ ID NO: 155) |
| smOPT | aATTTTTGGAGca (SEQ ID NO: 49) |
| ms-eSL | Ggttttctgacctccgtcggaaaacccct (SEQ ID NO: 14) |
| U7 | Cccaatttcactggtctacaatgaaagcaaaacagttctcttccccgctccccggtgtgtg |
| termination | agaggggctttgatccttctctggtttcctaggaaacgcgtatgtg (SEQ ID NO: 81) |
| U1 promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTC (SEQ ID NO: 66) |
| 5′ISD | ggagt |
| ADAR | GGTGTCGAGAAGAGGAGAACAATATGCTAAATGTTGTTCTCGTCTCCTCG |
| recruiting | ACACC (SEQ ID NO: 88) |
| sequence | |
| Non- | Gctacgtcaataggcactaacttattggcgctggtgaacggacttcctctcgagta |
| Targeting | ccagaagatgactacaaaactcctttccattgcgagtatcggagtctgg |
| Sequence | (SEQ ID NO: 155) |
| smOPT | aATTTTTGGAGca (SEQ ID NO: 49) |
| ms-eSLv | Ggttttctgacctccgtcggaaaacccct (SEQ ID NO: 14) |
| U1 | GTTTACTTGGTTTTAAAAATAGCTTGCACTAGCGATACGGAATATGGTTAT |
| termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |
| ITR | Aggaacccctagtgatggagttggccactccctctctgcgcgctcgctcgctc |
| | actgaggccgggcgaccaaaggtcgcccgacgcccgggctttgcccgggggcc |
| | tcagtgagcgagcgagcgcgcagctgcctgcagg (SEQ ID NO: 90) |

In one embodiment the A05322 vector comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 180.

A05488: Stuffer-scAAV-1x_aR mU7_ISD_IDUA(W402X) mature-mRNA 105 bp

| ITR | Ctgcgcgctcgctcgctcactgaggccgcccgggcaaagcccgggcgtcggg |
|---|---|
| | cgacctttggtcgcccggcctcagtgagcgagcgagcgcgcagagagggagt |
| | ggggtt (SEQ ID NO: 89) |
| U1 promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTC (SEQ ID NO: 66) |
| 5′ISD | ggagt |
| Targeting | GCCCACCGTGTGGTTGCTGTCCAGGACGGTCCCGGCCTGCGACACTTCGG |
| Sequence | CCCAGAGCTGCTCCTCATCCAGCAGCGCCAGCAGCCCCATGGCCGTGAGC |
| | ACCGG (SEQ ID NO: 153) |
| smOPT | aATTTTTGGAGca (SEQ ID NO: 49) |
| ms-eSL | Ggttttctgacctccgtcggaaaacccct (SEQ ID NO: 14) |
| U1 | GTTTACTTGGTTTTAAAAATAGCTTGCACTAGCGATACGGAATATGGTTAT |
| termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |
| ITR | Aggaacccctagtgatggagttggccactccctctctgcgcgctcgctcgct |
| | cactgaggccgggcgaccaaaggtcgcccgacgcccgggctttgcccgggcg |
| | gcctcagtgagcgagcgagcgcgcagctgcctgcagg (SEQ ID NO: |
| | 90) |

In one embodiment the A05488 vector comprises, consists essentially of, or consists of a nucleic acid sequence at least 65%, 70%, 75%, 80%, 85%, 90%, 95%, 96%, 97%, 98%, 99% or 10000 (or any percentage in between) identical to SEQ ID NO: 181.

A05489: Stuffer-scAAV-1x_aR mU7_ISD_IDUA
(W402X) mature-mRNA 105 bp + ADAR

| | |
|---|---|
| ITR | Ctgcgcgctcgctcgctcactgaggccgcc |
| | cgggcaaagcccgggcgtcgggcgaccttt |
| | ggtcgcccggcctcagtgagcgagcgagcg |
| | cgcagagagggagtggggtt |
| | (SEQ ID NO: 89) |
| U1 promoter | TTGTTCCTCTTAGTGTTAATTCACACTAAA |
| | GACTGTGCATCCGACTCCTACATTTATGAA |
| | AGTAAATGCCTGTTGTTAGAACAAAAAAGG |
| | CTACAGAACAAAAAACAAAGCGAAATACCA |
| | TCTGCTTTAGGTTCAGTGTGGTATTTTCCC |
| | GCTGACAGGGAGGCGGGTTTTTGGGTACAG |
| | GAAACGAGTCACTATGGAGGCGGTACTATG |
| | TAGATGAGAATTCAGGTGCAAACTGGGAAA |
| | AGCAACTGCTTCCAAATATTTGTGATTTTT |
| | ACAGTGTAGTTTTGGAAAAACTCTTAGCCT |
| | ACCAATTCTTCTAAGTGTTTTAAAATGTGG |
| | GAGCCAGTACACATGAAGTTATAGAGTGTT |
| | TTAATGAGGCTTAAATATTTACCGTAACTA |
| | TGAAATGCTACGCATATCATGCTGTTCAGG |
| | CTCCGTGGCCACGCAACTC |
| | (SEQ ID NO: 66) |
| 5′ISD | ggagt |
| ADAR | GGTGTCGAGAAGAGGAGAACAATATGCTAA |
| recruiting | ATGTTGTTCTCGTCTCCTCGACACC (SEQ |
| sequence | ID NO: 88) |
| Targeting | GCCCACCGTGTGGTTGCTGTCCAGGACGGT |
| Sequence | CCCGGCCTGCGACACTTCGGCCCAGAGCTG |
| | CTCCTCATCCAGCAGCGCCAGCAGCCCCAT |
| | GGCCGTGAGCACCGG |
| | (SEQ ID NO: 153) |
| smOPT | aATTTTTGGAGca (SEQ ID NO: 49) |
| ms-eSL | Ggttttctgacctccgtcggaaaacccct |
| | (SEQ ID NO: 14) |
| U1 | GTTTACTTGGTTTTAAAAATAGCTTGCACT |
| termination | AGCGATACGGAATATGGTTATTAGGTTTGT |
| | TAGGCATCATGTCGTGTCTTACTATAGAAA |
| | AATAACGTAGTGTTCATTTTAGCCTGCCTG |
| | TATGTGTTAATTTGTCCTTATTGCGCATTG |
| | TTCTTGTTAAGTCTTCTGTAAGGAGTTGCG |
| | GGTTTCAAACTGTCAGTCTGAGAGCA |
| | (SEQ ID NO: 80) |
| ITR | Aggaacccctagtgatggagttggccactc |
| | cctctctgcgcgctcgctcgctcactgagg |
| | ccgggcgaccaaaggtcgcccgacgcccgg |
| | gctttgcccgggggcctcagtgagcgagcg |
| | agcgcgcagctgcctgcagg |
| | (SEQ ID NO: 90) |

In one embodiment the A05489 vector comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 182.

A05490: Stuffer-scAAV-1x aR mU7_ISD_IDUA(W402X)
mature-mRNA_ADAR_20 bp_3xRS

| | |
|---|---|
| ITR | Ctgcgcgctcgctcgctcactgaggccgcc |
| | cgggcaaagcccgggcgtcgggcgaccttt |
| | ggtcgcccggcctcagtgagcgagcgagcg |
| | cgcagagagggagtggggtt |
| | (SEQ ID NO: 89) |
| U1 promoter | TTGTTCCTCTTAGTGTTAATTCACACTAAA |
| | GACTGTGCATCCGACTCCTACATTTATGAA |
| | AGTAAATGCCTGTTGTTAGAACAAAAAAGG |
| | CTACAGAACAAAAAACAAAGCGAAATACCA |
| | TCTGCTTTAGGTTCAGTGTGGTATTTTCCC |
| | GCTGACAGGGAGGCGGGTTTTTGGGTACAG |
| | GAAACGAGTCACTATGGAGGCGGTACTATG |
| | TAGATGAGAATTCAGGTGCAAACTGGGAAA |
| | AGCAACTGCTTCCAAATATTTGTGATTTTT |
| | ACAGTGTAGTTTTGGAAAAACTCTTAGCCT |
| | ACCAATTCTTCTAAGTGTTTTAAAATGTGG |
| | GAGCCAGTACACATGAAGTTATAGAGTGTT |
| | TTAATGAGGCTTAAATATTTACCGTAACTA |
| | TGAAATGCTACGCATATCATGCTGTTCAGG |
| | CTCCGTGGCCACGCAACTC |
| | (SEQ ID NO: 66) |
| 5′ISD | ggagt |
| ADAR | GGTGTCGAGAAGAGGAGAACAATATGCTAA |
| recruiting | ATGTTGTTCTCGTCTCCTCGACACC |
| sequence | (SEQ ID NO: 88) |
| Targeting | TTCGGCCcAGAGCTGCTCCTAAAGGCCGTG |
| Sequence | AGCACCGGAAACGTGCGGCGGGCGGGAAAG |
| | CGCGAAGGGGTGCGGGTGG |
| | (SEQ ID NO: 154) |
| smOPT | aATTTTTGGAGca |
| | (SEQ ID NO: 49) |
| ms-eSL | Ggttttctgacctccgtcggaaaacccct |
| | (SEQ ID NO: 14) |
| U1 | GTTTACTTGGTTTTAAAAATAGCTTGCACT |
| termination | AGCGATACGGAATATGGTTATTAGGTTTGT |
| | TAGGCATCATGTCGTGTCTTACTATAGAAA |
| | AATAACGTAGTGTTCATTTTAGCCTGCCTG |
| | TATGTGTTAATTTGTCCTTATTGCGCATTG |
| | TTCTTGTTAAGTCTTCTGTAAGGAGTTGCG |
| | GGTTTCAAACTGTCAGTCTGAGAGCA |
| | (SEQ ID NO: 80) |
| ITR | Aggaacccctagtgatggagttggccactc |
| | cctctctgcgcgctcgctcgctcactgagg |
| | ccgggcgaccaaaggtcgcccgacgcccgg |
| | gctttgcccgggggcctcagtgagcgagcg |
| | agcgcgcagctgcctgcagg |
| | (SEQ ID NO: 90) |

In one embodiment the A05490 vector comprises, consists essentially of, or consists of a nucleic acid sequence at least 65%, 70%, 75%, 80%, 85%, 90%, 95%, 96%, 97%, 98%, 99% or 10000 (or any percentage in between) identical to SEQ ID NO: 183.

A05492: Stuffer-scAAV-1x_aR mU7_ISD_IDUA
(W402X) Non-Targeting

| | |
|---|---|
| ITR | Ctgcgcgctcgctcgctcactgaggccgcc |
| | cgggcaaagcccgggcgtcgggcgaccttt |
| | ggtcgcccggcctcagtgagcgagcgagcg |
| | cgcagagagggagtggggtt |
| | (SEQ ID NO: 89) |
| U1 promoter | TTGTTCCTCTTAGTGTTAATTCACACTAAA |
| | GACTGTGCATCCGACTCCTACATTTATGAA |
| | AGTAAATGCCTGTTGTTAGAACAAAAAAGG |
| | CTACAGAACAAAAAACAAAGCGAAATACCA |
| | TCTGCTTTAGGTTCAGTGTGGTATTTTCCC |
| | GCTGACAGGGAGGCGGGTTTTTGGGTACAG |
| | GAAACGAGTCACTATGGAGGCGGTACTATG |
| | TAGATGAGAATTCAGGTGCAAACTGGGAAA |
| | AGCAACTGCTTCCAAATATTTGTGATTTTT |
| | ACAGTGTAGTTTTGGAAAAACTCTTAGCCT |
| | ACCAATTCTTCTAAGTGTTTTAAAATGTGG |
| | GAGCCAGTACACATGAAGTTATAGAGTGTT |
| | TTAATGAGGCTTAAATATTTACCGTAACTA |
| | TGAAATGCTACGCATATCATGCTGTTCAGG |
| | CTCCGTGGCCACGCAACTC |
| | (SEQ ID NO: 66) |
| 5′ISD | ggagt |
| ADAR | GGTGTCGAGAAGAGGAGAACAATATGCTAA |
| recruiting | ATGTTGTTCTCGTCTCCTCGACACC |
| sequence | (SEQ ID NO: 88) |
| Non- | Gctacgtcaataggcactaacttattggcg |
| | ctggtgaacggacttcctctcgagtaccag |
| Targeting | aagatgactacaaaactcctttccattgcg |
| Sequence | agtatcggagtctgg |
| | (SEQ ID NO: 155) |
| smOPT | aATTTTTGGAGca (SEQ ID NO: 49) |
| ms-eSL | Ggttttctgacctccgtcggaaaacccct |
| | (SEQ ID NO: 14) |
| U1 | GTTTACTTGGTTTTAAAAATAGCTTGCACT |
| termination | AGCGATACGGAATATGGTTATTAGGTTTGT |
| | TAGGCATCATGTCGTGTCTTACTATAGAAA |
| | AATAACGTAGTGTTCATTTTAGCCTGCCTG |
| | TATGTGTTAATTTGTCCTTATTGCGCATTG |
| | TTCTTGTTAAGTCTTCTGTAAGGAGTTGCG |
| | GGTTTCAAACTGTCAGTCTGAGAGCA |
| | (SEQ ID NO: 80) |
| ITR | Aggaacccctagtgatggagttggccactc |
| | cctctctgcgcgctcgctcgctcactgagg |
| | ccgggcgaccaaaggtcgcccgacgcccgg |
| | gctttgcccgggggcctcagtgagcgagcg |
| | agcgcgcagctgcctgcagg |
| | (SEQ ID NO: 90) |

In one embodiment the A05492 vector comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 184.

MBNL1 (Muscleblind-like protein 7) is an RNA-binding protein sequestered to pathologic CUG repeats, resulting in the decrease of functional MBNL1 in Myotonic Dystrophy (DM1). Targeting and editing MBNL1 binding sites, results in an increase of MBNL1 expression thereby treating DM1.

Exemplary MBNL1 editing constructs are as follows:

P05802: MBNL1-mir30a-5p U7-snRNA; 71 nt + RD; A1G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | tggcaattttaaatacaaggtgcacctttgttatttgCaaaccttgaaaga |
| seq. | aataaacttatttttaaaaa (SEQ ID NO: |
| | 156) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05803: MBNL1-mir30a-5p U7-snRNA; 71 nt + RD; A2G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | tggcaattttaaatacaaggtgcacctttgttattCgtaaaccttgaaa |
| seq. | gaaataaacttatttttaaaaa (SEQ ID NO: |
| | 157) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05804: MBNL1-mir30a-5p U7-snRNA; 71 nt + RD; A1G + A2G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | tggcaattttaaatacaaggtgcacctttgttattCgCaaaccttgaaa |
| seq. | gaaataaacttatttttaaaaa (SEQ ID NO: |
| | 158) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05805: MBNL1-mir30a-5p U7-snRNA; 151 nt + RD; A1G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | ttaaacaaaaatgactgtgcatgcacgctctcatctataatggcaatttt |
| seq. | aaatacaaggtgcacctttgttatttgCaaaccttgaaagaaataaactt |
| | atttttaaaaaattatatcttggtctacagacgtaccaatacataataga |
| | a (SEQ ID NO: 159) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05806: MBNL1-mir30a-5p U7-snRNA; 151 nt + RD; A2G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | ttaaacaaaaatgactgtgcatgcacgctctcatctataatggcaatttta |
| seq. | aatacaaggtgcacctttgttattCgtaaaccttgaaagaaataaacttat |
| | ttttaaaaaattatatcttggtctacagacgtaccaatacataatagaa |
| | (SEQ ID NO: 160) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05807: MBNL1-mir30a-5p U7-snRNA; 151 nt + RD; A1G + A2G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | ttaaacaaaaatgactgtgcatgcacgctctcatctataatggcaatttta |
| seq. | aatacaaggtgcacctttgttattCgCaaaccttgaaagaaataaacttat |
| | ttttaaaaaattatatcttggtctacagacgtaccaatacataatagaa |
| | (SEQ ID NO: 161) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05808: MBNL1-mir30a-3p U7-snRNA; 71 nt; A1G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | tggcaattttaaatacaaggtgcacctttgttatttgCaaaccttgaaaga |
| seq. | aataaacttatttttaaaaa (SEQ ID NO: |
| | 156) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05809: MBNL1-mir30a-3p U7-snRNA; 71 nt; A2G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | tggcaattttaaatacaaggtgcacctttgttattCgtaaaccttgaaaga |
| seq. | aataaacttatttttaaaaa (SEQ ID NO: |
| | 157) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A(SEQ ID NO: 80) |

P05810: MBNL1-mir30a-3p U7-snRNA; 71 nt; A1G + a2G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | tggcaattttaaatacaaggtgcacctttgttattCgCaaaccttgaaaga |
| seq. | aataaacttatttttaaaaa (SEQ ID NO: |
| | 158) |
| smOPT | aATTTTTGGAGca (SEQ ID NO: 49) |
| ms-short-eSL | Ggttttctgacctccgtoggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05811: MBNL1-mir30a-3p U7-snRNA; 151 nt; A1G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | ttaaacaaaaatgactgtgcatgcacgctctcatctataatggcaattttaa |
| seq. | atacaaggtgcacctttgttatttgCaaaccttgaaagaaataaacttattt |
| | ttaaaaaattatatcttggtctacagacgtaccaatacataatagaa |
| | (SEQ ID NO: 159) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05812: MBNL1-mir30a-3p U7-snRNA; 151 nt; A2G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | ttaaacaaaaatgactgtgcatgcacgctctcatctataatggcaatttta |
| seq. | aatacaaggtgcacctttgttattCgtaaaccttgaaagaaataaacttat |
| | ttttaaaaaattatatcttggtctacagacgtaccaatacataatagaa |
| | (SEQ ID NO: 160) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05813: MBNL1-mir30a-3p U7-snRNA; 151 nt; A1G + A2G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | ttaaacaaaaatgactgtgcatgcacgctctcatctataatggcaatttta |
| seq. | aatacaaggtgcacctttgttattCgCaaaccttgaaagaaataaacttat |
| | ttttaaaaaattatatcttggtctacagacgtaccaatacataatagaa |
| | (SEQ ID NO: 161) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05993: MBNL1-mir23a-3p U7-snRNA + RD; 71 nt; A1G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | tggatacactaattcttttaaacaagtgcccattaCcacattttgcatttt |
| seq. | tgagtgcttctgagttgaaa (SEQ ID NO: 162) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05994: MBNL1-mir23a-3p U7-snRNA; 71 nt + RD; A2G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | tggatacactaattcttttaaacaagtgcccattatcacaCtttgcattt |
| seq. | ttgagtgcttctgagttgaaa (SEQ ID NO: 163) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05995: MBNL1-mir23a-3p U7-snRNA; 71 nt + RD; A3G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | tggatacactaattcttttaaacaagtgcccattatcacatCttgcatt |
| seq. | tttgagtgcttctgagttgaaa (SEQ ID NO: |
| | 164) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05996: MBNL1-mir23a-3p U7-snRNA; 71 nt + RD; A1G + A2G + A3G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | tggatacactaattcttttaaacaagtgcccattaCcacaCCttgcattt |
| seq. | ttgagtgcttctgagttgaaa (SEQ ID NO: |
| | 165) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05997: MBNL1-mir23a-3p U7-snRNA; 111 nt + RD; A1G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | taaccagctggagtgaaggctggatacactaattcttttaaacaagtgccc |
| seq. | attaCcacattttgcatttttgagtgcttctgagt |
| | tgaaaattaaaaccatgaaatttga (SEQ ID NO: 166) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05998: MBNL1-mir23a-3p U7-snRNA; 111 nt + RD; A2G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | taaccagctggagtgaaggctggatacactaattcttttaaacaagtgcc |
| seq. | cattatcacaCtttgcatttttgagtgcttctgagt |
| | tgaaaattaaaaccatgaaatttga (SEQ ID NO: 167) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P05999: MBNL1-mir23a-3p U7-snRNA; 111 nt + RD; A3G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | taaccagctggagtgaaggctggatacactaattcttttaaacaagtgc |
| seq. | ccattatcacatCttgcatttttgagtgcttctgagt |
| | tgaaaattaaaaccatgaaatttga (SEQ ID NO: 168) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06000: MBNL1-mir23a-3p U7-snRNA; 111 nt + RD; A1G + A2G + A3G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | taaccagctggagtgaaggctggatacactaattcttttaaacaagtgcc |
| seq. | cattaCcacaCCttgcatttttgagtgcttctga |
| | gttgaaaattaaaaccatgaaatttga (SEQ ID NO: 169) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06001: MBNL1-mir23a-3p U7-snRNA; 151 nt + RD; A1G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | gctgataagtgcaacatttttaaccagctggagtgaaggctggatacacta |
| seq. | attcttttaaacaagtgcccattaCcacattttgcatttttgagtgcttct |
| | gagttgaaaattaaaaccatgaaatttgaactgcgttttatttttcacc |
| | (SEQ ID NO: 170) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06002: MBNL1-mir23a-3p U7-snRNA; 151 nt + RD; A2G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | gctgataagtgcaacatttttaaccagctggagtgaaggctggatacact |
| seq. | aattcttttaaacaagtgcccattatcacaCtttgcatttttgagtgctt |
| | ctgagttgaaaattaaaaccatgaaatttgaactgcgttttatttttcacc |
| | (SEQ ID NO: 171) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06003: MBNL1-mir23a-3p U7-snRNA; 151 nt + RD; A3G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | gctgataagtgcaacatttttaaccagctggagtgaaggctggatacacta |
| seq. | attcttttaaacaagtgcccattatcacatCttgcatttttgagtgcttct |
| | gagttgaaaattaaaaccatgaaatttgaactgcgttttatttttcacc |
| | (SEQ ID NO: 172) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06004: MBNL1-mir23a-3p U7-snRNA; 151 nt + RD; A1G + A2G + A3G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| ADAR recr. | GGTGTCGAGAAGAGGAGAACAATATGCTAAATGTTGTTCTCGTCTCCTCG |
| | ACACC (SEQ ID NO: 88) |
| Targeting- | gctgataagtgcaacatttttaaccagctggagtgaaggctggatacact |
| seq. | aattcttttaaacaagtgcccattaCcacaCCttgcatttttgagtgctt |
| | ctgagttgaaaattaaaaccatgaaatttgaactgcgttttatttttcac |
| | c (SEQ ID NO: 173) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06121: MBNL1-mir23a-3p U7-snRNA; 71 nt; A1G

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | tggatacactaattcttttaaacaagtgcccattaCcacattttgcattttt |
| seq. | gagtgcttctgagttgaaa (SEQ ID NO: 162) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06122: MBNL1-mir23a-3p U7-snRNA; 71 nt; A2G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | tggatacactaattcttttaaacaagtgcccattatcacaCtttgcatttt |
| seq. | tgagtgcttctgagttgaaa (SEQ ID NO: 163) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06123: MBNL1-mir23a-3p U7-snRNA; 71 nt; A3G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | tggatacactaattcttttaaacaagtgcccattatcacatCttgcatttt |
| seq. | tgagtgcttctgagttgaaa (SEQ ID NO: 164) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06124: MBNL1-mir23a-3p U7-snRNA; 71 nt; A1G + A2G + A3G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | tggatacactaattcttttaaacaagtgcccattaCcacaCCttgcattt |
| seq. | ttgagtgcttctgagttgaaa (SEQ ID NO: |
| | 165) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06125: MBNL1-mir23a-3p U7-snRNA; 111 nt; A1G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | taaccagctggagtgaaggctggatacactaattcttttaaacaagtgccc |
| seq. | attaCcacattttgcatttttgagtgcttctgagt |
| | tgaaaattaaaaccatgaaatttga (SEQ ID NO: 166) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06126: MBNL1-mir23a-3p U7-snRNA; 111 nt; A2G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | taaccagctggagtgaaggctggatacactaattcttttaaacaagtgcc |
| seq. | cattatcacaCtttgcatttttgagtgcttctgagt |
| | tgaaaattaaaaccatgaaatttga (SEQ ID NO: 167) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06127: MBNL1-mir23a-3p U7-snRNA; 111 nt; A3G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | taaccagctggagtgaaggctggatacactaattcttttaaacaagtgcc |
| seq. | cattatcacatCttgcatttttgagtgcttctgagt |
| | tgaaaattaaaaccatgaaatttga (SEQ ID NO: 168) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06128: MBNL1-mir23a-3p U7-snRNA; 111 nt; A1G + A2G + A3G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | taaccagctggagtgaaggctggatacactaattcttttaaacaagtgcc |
| seq. | cattaCcacaCCttgcatttttgagtgcttctga |
| | gttgaaaattaaaaccatgaaatttga (SEQ ID NO: 169) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06129: MBNL1-mir23a-3p U7-snRNA; 151 nt; A1G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | gctgataagtgcaacatttttaaccagctggagtgaaggctggatacacta |
| seq. | attcttttaaacaagtgcccattaCcacattttgcatttttgagtgcttct |
| | gagttgaaaattaaaaccatgaaatttgaactgcgttttatttttcacc |
| | (SEQ ID NO: 170) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06130: MBNL1-mir23a-3p U7-snRNA; 151 nt + A2G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | gctgataagtgcaacatttttaaccagctggagtgaaggctggatacact |
| seq. | aattcttttaaacaagtgcccattatcacaCtttgcatttttgagtgctt |
| | ctgagttgaaaattaaaaccatgaaatttgaactgcgttttatttttcac |
| | c (SEQ ID NO: 171) |
| smOPT | aATTTTTGGAGca (SEQ ID NO: 49) |
| ms-short-eSL | Ggttttctgacctccgtoggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06131: MBNL1-mir23a-3p U7-snRNA; 151 nt; A3G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | gctgataagtgcaacatttttaaccagctggagtgaaggctggatacact |
| seq. | aattcttttaaacaagtgcccattatcacatCttgcatttttgagtgctt |
| | ctgagttgaaaattaaaaccatgaaatttgaactgcgttttatttttca |
| | cc (SEQ ID NO: 172) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

P06132: MBNL1-mir23a-3p U7-snRNA; 151 nt; A1G + A2G + A3G; P04861 backbone

| | |
|---|---|
| U1 Promoter | TTGTTCCTCTTAGTGTTAATTCACACTAAAGACTGTGCATCCGACTCCTAC |
| | ATTTATGAAAGTAAATGCCTGTTGTTAGAACAAAAAAGGCTACAGAACAA |
| | AAAACAAAGCGAAATACCATCTGCTTTAGGTTCAGTGTGGTATTTTCCCGC |
| | TGACAGGGAGGCGGGTTTTTGGGTACAGGAAACGAGTCACTATGGAGGCG |
| | GTACTATGTAGATGAGAATTCAGGTGCAAACTGGGAAAAGCAACTGCTTC |
| | CAAATATTTGTGATTTTTACAGTGTAGTTTTGGAAAAACTCTTAGCCTACC |
| | AATTCTTCTAAGTGTTTTAAAATGTGGGAGCCAGTACACATGAAGTTATAG |
| | AGTGTTTTAATGAGGCTTAAATATTTACCGTAACTATGAAATGCTACGCAT |
| | ATCATGCTGTTCAGGCTCCGTGGCCACGCAACTCaa (SEQ ID NO: 66) |
| Targeting- | gctgataagtgcaacatttttaaccagctggagtgaaggctggatacact |
| seq. | aattcttttaaacaagtgcccattaCcacaCCttgcatttttgagtgcttc |
| | tgagttgaaaattaaaaccatgaaatttgaactgcgttttatttttcac |
| | c (SEQ ID NO: 173) |
| smOPT | aATTTTTGGAGca (SEQ ID NO: 49) |
| ms-short-eSL | Ggttttctgacctccgtcggaaaacc (SEQ ID NO: 16) |
| U1 | GTTTACTTGGTTTTAAAAATAGCTTGCACTAGCGATACGGAATATGGTTAT |
| Termination | TAGGTTTGTTAGGCATCATGTCGTGTCTTACTATAGAAAAATAACGTAGTG |
| | TTCATTTTAGCCTGCCTGTATGTGTTAATTTGTCCTTATTGCGCATTGTTCT |
| | TGTTAAGTCTTCTGTAAGGAGTTGCGGGTTTCAAACTGTCAGTCTGAGAGC |
| | A (SEQ ID NO: 80) |

Additional Exemplary RNA-targeting constructs of the disclosure are as follows:

P03441 pcDNA3.1_U7_U7-ADAR-ISD-recruiting-mCherry-Stop 70 (SEQ ID NO: 102)

| | |
|---|---|
| 5′ISD | Ggagt |
| ADAR | GGTGTCGAGAAGAGGAGAACAATATGCTAAATGTTGTTCTCGTCTCC |
| recr. | TCGACACC (SEQ ID NO: 88) |
| targeting | tgaacagctcctcgcccttgctcactggcagagccctcCagcatcgcga |
| seq | gcaggcgctgcctcctccgcc |
| | (SEQ ID NO: 151) |
| SmOPT | AATTTTTGGAGTA (SEQ ID NO: 92) |
| (SmBD) | |
| eSL | Ggctttctggctccttaccggaaagcc (SEQ ID NO: 1) |

P03442 pcDNA3.1_U7_U7-ADAR-ISD recruiting-mCherry-Stop 151 (SEQ ID NO: 103)

| | |
|---|---|
| 5′ISD | ggagt |
| ADAR recr. | GGTGTCGAGAAGAGGAGAACAATATGCTAAATGTTGTTCTCGTC |
| | TCCTCGACACC (SEQ ID NO: 88) |
| targeting seq | tcgccgtccagctcgaccaggatgggcaccaccccggtgaacagctc |
| | ctcgcccttgctcactggcagagccctcCagcatcgcgagcaggcgc |
| | tgcctcctccgccgctgcctcctccgccgctgcctcctccgccc |
| | tgcagcttgtaca (SEQ ID NO: 152) |
| SmOPT | AATTTTTGGAGTA (SEQ ID NO: 92) |
| eSL | Ggctttctggctccttaccggaaagcc (SEQ ID NO: 1) |

P03501 pcDNA3.1_U7_U7-ADAR-ISD recruiting-nt 70 (SEQ ID NO: 104)

| | |
|---|---|
| 5′ISD | ggagt |
| ADAR | GGTGTCGAGAAGAGGAGAACAATATGCTAAATGTTGTTCTCGTCTCC |
| recr. | TCGACACC (SEQ ID NO: 88) |
| Non- | Aaaccgagggatcataggggactgaatccaccattcttctcccaatc |
| targeting | cctgcaactccttcttcccctgc (SEQ |
| seq | ID NO: 174) |
| SmOPT | AATTTTTGGAGTA (SEQ ID NO: 92) |
| eSL | Ggctttctggctccttaccggaaagcc (SEQ ID NO: 1) |

P03502 pcDNA3.1_U7_U7-ADAR-ISD recruiting-nt 151 (SEQ ID NO: 105)

| | |
|---|---|
| 5′ISD | ggagt |
| ADAR recr. | GGTGTCGAGAAGAGGAGAACAATATGCTAAATGTTGTTCTCGTC |
| | TCCTCGACACC (SEQ ID NO: 88) |
| Non-targeting | Actacagttgctccgatatttaggctacgtcaataggcact |
| sequence | aacttattggcgctggtgaacggacttcctctcgagtacca |
| | gaagatgactacaaaactcctttccattgcgagtatcggag |
| | tctggctcagtttggccaggga |
| | ggcact (SEQ ID NO: 155) |
| SmOPT | AATTTTTGGAGTA (SEQ ID NO: 92) |
| eSL | Ggctttctggctccttaccggaaagcc (SEQ ID NO: 1) |

P03503 pcDNA3.1_U7_U7-ADAR-ISD recruiting-mCherry-Stop 70 (SEQ ID NO: 106)

| | |
|---|---|
| ADAR | GGTGTCGAGAAGAGGAGAACAATATGCTAAATGTTGTTCTCGTCTCC |
| recr. | TCGACACC (SEQ ID NO: 88) |
| 5′ targeting | tgaacagctcctcgcccttgctcactggcagagccctcCagcatcgcgagca |
| seq | ggcgctgcctcctccgcc(SEQID NO: 151) |
| SmOPT | AATTTTTGGAGTA (SEQ ID NO: 92) |
| eSL | Ggctttctggctccttaccggaaagcc (SEQ ID NO: 1) |

P03504 pcDNA3.1 U7_U7-ADAR-recruiting-mCherry-Stop 151 (SEQ ID NO: 107)

| | |
|---|---|
| ADAR recr. | GGTGTCGAGAAGAGGAGAACAATATGCTAAATGTTGTTCTCGTC |
| | TCCTCGACACC (SEQ ID NO: 88) |
| targeting | tcgccgtccagctcgaccaggatgggcaccaccccggtgaacag |
| seq | ctcctcgcccttgctcactggcagagccctcCagcatcgcgagc |
| | aggcgctgcctcctccgccgctgcctcctccgccgctgcctcct |
| | ccgccctgcagcttgtaca (SEQ ID NO: 152) |
| SmOPT | AATTTTTGGAGTA (SEQ ID NO: 92) |
| eSL | Ggctttctggctccttaccggaaagcc (SEQ ID NO: 1) |

P03633 pcDNA3.1_U7_U7-ADAR recruiting-nt 70 (SEQ ID NO: 108)

| | |
|---|---|
| ADAR | GGTGTCGAGAAGAGGAGAACAATATGCTAAATGTTGTTCTCGTCTCC |
| recr. | TCGACACC (SEQ ID NO: 88) |
| Non- | Aaaccgagggatcataggggactgaatccaccattcttctcccaat |
| targeting | ccctgcaactccttcttcccctgc (SEQ |
| seq | ID NO: 174) |
| SmOPT | AATTTTTGGAGTA (SEQ ID NO: 92) |
| eSL | Ggctttctggctccttaccggaaagcc (SEQ ID NO: 1) |

P03434 pcDNA3.1_U7_U7-ADAR recruiting-nt 151 (SEQ ID NO: 109)

| | |
|---|---|
| ADAR recr. | GGTGTCGAGAAGAGGAGAACAATATGCTAAATGTTGTTCTCGTC |
| | TCCTCGACACC (SEQ ID NO: 88) |
| Non-targeting | Actacagttgctccgatatttaggctacgtcaataggcactaa |
| sequence | cttattggcgctggtgaacggacttcctctcgagtaccagaaga |
| | tgactacaaaactcctttccattgcgagtatcggagtctggctc |
| | agtttggccagggaggcact (SEQ ID NO: 155) |
| SmOPT | AATTTTTGGAGTA (SEQ ID NO: 92) |
| eSL | Ggctttctggctccttaccggaaagcc (SEQ ID NO: 1) |

P03437 pcDNA3.1_U7_U7-5′ISD-ADAR-mCherry-Stop 70 (SEQ ID NO: 110)

| | |
|---|---|
| 5′ISD | ggagt |
| targeting seq | tgaacagctcctcgcccttgctcactggcagagccctcCagcatcg |
| | cgagcaggcgctgcctcctccgcc |
| | (SEQ ID NO: 151) |
| SmOPT | AATTTTTGGAGTA (SEQ ID NO: 92) |
| eSL | Ggctttctggctccttaccggaaagcc (SEQ ID NO: 1) |

P03438 pcDNA3.1_U7_U7-5′ISD-ADAR-mCherry-Stop 151 (SEQ ID NO: 111)

| | |
|---|---|
| 5′ISD | ggagt |
| targeting seq | tcgccgtccagctcgaccaggatgggcaccaccccggtgaac |
| | agctcctcgcccttgctcactggcagagccctcCagcatcgc |
| | gagcaggcgctgcctcctccgccgctgcctcctccgccgctg |
| | cctcctccgccctgcagcttgtaca |
| | (SEQ ID NO: 152) |
| SmOPT | AATTTTTGGAGTA (SEQ ID NO: 92) |
| eSL | Ggctttctggctccttaccggaaagcc (SEQ ID NO: 1) |

P03439 pcDNA3.1_U7_U7-5′ISD-ADAR-nt 70 (SEQ ID NO: 112)

| | |
|---|---|
| 5′ISD | ggagt |
| Non-targeting | Aaaccgagggatcataggggactgaatccaccattcttctc |
| seq | ccaatccctgcaactccttcttcccctgc |
| | (SEQ ID NO: 174) |
| SmOPT | AATTTTTGGAGTA (SEQ ID NO: 92) |
| eSL | Ggctttctggctccttaccggaaagcc (SEQ ID NO: 1) |

P03440 pcDNA3.1_U7_U7-5′ISD-ADAR-nt 151 (SEQ ID NO: 113)

| | |
|---|---|
| 5′ISD | ggagt |
| Non-targeting | Actacagttgctccgatatttaggctacgtcaataggcacta |
| sequence | acttattggcgctggtgaacggacttcctctcgagtaccagaa |
| | gatgactacaaaactcctttccattgcgagtatcggagtctgg |
| | ctcagtttggccagggaggcact (SEQ ID NO: 155) |
| SmOPT | AATTTTTGGAGTA (SEQ ID NO: 92) |
| eSL | Ggctttctggctccttaccggaaagcc (SEQ ID NO: 1) |

P03778 pcDNA3.1_U7_U7-ADAR-ISD-recruiting-mCherry-Stop 70 No mismatch
(SEQ ID NO: 114)

| | |
|---|---|
| 5′ISD | ggagt |
| ADAR | GGTGTCGAGAAGAGGAGAACAATATGCTAAATGTTGTTCTCGTCTC |
| recr. | CTCGACACC (SEQ ID NO: 88) |
| 5′ | tgaacagctcctcgcccttgctcactggcagagccctcTag |
| targeting | catcgcgagcaggcgctgcctcctccgcc |
| seq | (SEQ ID NO: 42) |
| SmOPT | AATTTTTGGAGTA (SEQ ID NO: 92) |
| eSL | Ggctttctggctccttaccggaaagcc (SEQ ID NO: 1) |

P03779 pcDNA3.1_U7_U7-ADAR-ISD recruiting-mCherry-Stop 151 No mismatch
(SEQ ID NO: 115)

| | |
|---|---|
| 5′ISD | ggagt |
| ADAR | GGTGTCGAGAAGAGGAGAACAATATGCTAAATGTTGTTCTCGTCTC |
| recr. | CTCGACACC (SEQ ID NO: 88) |
| 5′ | tcgccgtccagctcgaccaggatgggcaccaccccggtgaaca |
| targeting | gctcctcgcccttgctcactggcagagccctcTagcatcgcga |
| seq | gcaggcgctgcctcctccgccgctgcctcctccgccgctgcct |
| | cctccgccctgcagc |
| | ttgtaca (SEQ ID NO: 175) |
| SmOPT | AATTTTTGGAGTA (SEQ ID NO: 92) |
| eSL | Ggctttctggctccttaccggaaagcc (SEQ ID NO: 1) |

P03780 pcDNA3.1_U7_U7-ADAR-recruiting-mCherry-Stop 70 No mismatch
(SEQ ID NO: 116)

| | |
|---|---|
| ADAR | GGTGTCGAGAAGAGGAGAACAATATGCTAAATGTTGTTCTCGTCTC |
| recr. | CTCGACACC (SEQ ID NO: 88) |
| 5′ | tgaacagctcctcgcccttgctcactggcagagccctcTagcat |
| targeting | cgcgagcaggcgctgcctcctccgcc |
| seq | (SEQ ID NO: 42) |
| SmOPT | AATTTTTGGAGTA (SEQ ID NO: 92) |
| eSL | Ggctttctggctccttaccggaaagcc (SEQ ID NO: 1) |

P03781 pcDNA3.1_U7_U7-ADAR-recruiting-mCherry-Stop 151 No mismatch
(SEQ ID NO: 117)

| | |
|---|---|
| ADAR | GGTGTCGAGAAGAGGAGAACAATATGCTAAATGTTGTTCTCGTCTC |
| recr. | CTCGACACC (SEQ ID NO: 88) |
| 5′ | tcgccgtccagctcgaccaggatgggcaccaccccggtgaacagctcctcgcc |
| targeting | cttgctcactggcagagccctcTagcatcgcgagcaggcgctgcctcctccgc |
| seq | cgctgcctcctccgccgctgcctcctccgccctgcagcttg |
| | taca (SEQ ID NO: 175) |
| SmOPT | AATTTTTGGAGTA (SEQ ID NO: 92) |
| eSL | Ggctttctggctccttaccggaaagcc (SEQ ID NO: 1) |

P03782 pcDNA3.1_U7_U7-5′ISD-ADAR-mCherry-Stop 70 No mismatch
(SEQ ID NO: 118)

| | |
|---|---|
| 5′ISD | ggagt |
| 5′ targeting | tgaacagctcctcgcccttgctcactggcagagccctcTagcatcg |
| seq | cgagcaggcgctgcctcctccgcc |
| | (SEQ ID NO: 42) |
| SmOPT | AATTTTTGGAGTA (SEQ ID NO: 92) |
| eSL | Ggctttctggctccttaccggaaagcc (SEQ ID NO: 1) |

P03783 pcDNA3.1_U7_U7-5′ISD-ADAR-mCherry-Stop 151 No mismatch
(SEQ ID NO: 119)

| | |
|---|---|
| 5′ISD | ggagt |
| 5′ | tcgccgtccagctcgaccaggatgggcaccaccccggtgaacag |
| targeting | ctcctcgcccttgctcactggcagagccctcTagcatcgcgagc |
| seq | aggcgctgcctcctccgccgctgcctcctccgccgctgcctcct |
| | ccgccctgcagcttgtaca |
| | (SEQ ID NO: 175) |
| SmOPT | AATTTTTGGAGTA (SEQ ID NO: 92) |
| eSL | Ggctttctggctccttaccggaaagcc (SEQ ID NO: 1) |

### Vectors

Also provided herein are vectors (e.g., recombinant expression vectors) comprising snRNA of the disclosure. In one embodiment, the snRNA is delivered in a vector.

In some embodiments of the compositions and methods of the disclosure, a vector comprises the snRNA. In some embodiments, the therapeutic snRNA is in a single or unitary vector.

In some embodiments of the compositions and methods of the disclosure, the RNA-binding snRNA systems capable of targeting G-to-A mutations, or other specific adenine in a target RNA sequence, are in a single vector.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector. Vectors are capable of autonomous replication in a host cell into which they are introduced such as e.g., bacterial vectors having a bacterial origin of replication and episomal mammalian vectors and other vectors such as, e.g., non-episomal mammalian vectors, are integrated into the genome of a host cell upon introduction into the host cell, and thereby are replicated along with the host genome.

In some embodiments, vectors such as e.g., expression vectors, are capable of directing the expression of genes to which they are operatively-linked. Common expression vectors are often in the form of plasmids. In some embodiments, recombinant expression vectors comprise a nucleic acid provided herein such as e.g., an snRNA in a form suitable for expression of a protein in a host cell. Recombinant expression vectors include one or more regulatory elements, which may be selected on the basis of the host cells to be used for expression, that is operatively-linked to the nucleic acid sequence to be expressed. Within a recombinant expression vector. “operably linked” is intended to mean that the nucleotide sequence of interest is linked to the regulatory element(s) in a manner that allows for expression of the nucleotide sequence such as e.g., in an in vitro transcription/translation system or in a host cell when the vector is introduced into the host cell.

Certain embodiments of a vector depend on factors such as the choice of the host cell to be transformed, and the level of expression desired. A vector can be introduced into host cells to thereby produce transcripts, proteins, or peptides, including fusion proteins or peptides, encoded by nucleic acids as described herein such as, e.g., snRNAs, CRISPR transcripts, proteins, enzymes, mutant forms thereof, fusion proteins thereof, etc.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. An “enhancer” is a region of DNA that can be bound by activating proteins to increase the likelihood or frequency of transcription.

In some embodiments of the compositions and methods of the disclosure, an expression vector, viral vector or non-viral vector provided herein, includes without limitation, vector elements such as a buffer sequence derived human genomic sequences downstream from an snRNA and as such will have the capability to encoding multiple snRNAs from a single construct.

In some embodiments, the snRNA constructs disclosed herein comprise bidirectional snRNA promoters to express snRNAs.

In another embodiment, the vector configurations can comprise linker(s), signal sequence(s), and/or tag(s).

### Viral Vectors

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

### Adeno-Associated Virus Vectors

In some embodiments, a vector described herein is an AAV viral vector. The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover. AAV infects many mammalian cells allowing the possibility of targeting many different tissues in vivo. Moreover. AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Recombinant AAV (rAAV) genomes of the invention may comprise, consist essentially of, or consist of a nucleic acid molecule encoding at least one esnRNA and one or more AAV ITRs flanking the nucleic acid molecule. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

An AAV vector described herein may comprise, consist essentially of, or consist of one or more nucleic acid molecules and one or more AAV ITRs. In some embodiments, the nucleic acid molecule encodes an esnRNA of the disclosure. Such AAV vectors can be replicated and packaged into infectious viral particles when present in a host cell that provides the functionality of rep and cap gene products, for example, by transfection of the host cell. In some embodiments, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing AAV vectors may also contain elements for manufacturing purposes, e.g., antibiotic resistance genes, origin of replication sequences etc., but these are not encapsidated and thus do not form part of the AAV particle.

In some embodiments, an AAV vector can comprise at least one nucleic acid encoding an snRNA of the disclosure. In some embodiments, an AAV vector can comprise at least one regulatory sequence. In some embodiments, an AAV vector can comprise at least one AAV inverted terminal (ITR) sequence. In some embodiments, an AAV vector can comprise a first ITR sequence and a second ITR sequence. In some embodiments, an AAV vector can comprise at least one promoter sequence. In some embodiments, an AAV vector can comprise at least one enhancer sequence. In some embodiments, an AAV vector can comprise at least one terminator sequence. In some embodiments, an AAV vector can comprise at least one polyA sequence. In some embodiments, an AAV vector can comprise at least one linker sequence. In some embodiments, an AAV vector can comprise at least one buffer sequence. In some embodiments, an AAV vector of the disclosure can comprise at least one nuclear localization signal, or nuclear export signal and/or both.

In some embodiments, an AAV vector can comprise a first AAV ITR sequence, a promoter sequence, an snRNA sequence, a terminator sequence and a second AAV ITR sequence. In some embodiments, an AAV vector can comprise, in the 5′ to 3′ direction, a first AAV ITR sequence, a promoter sequence, an snRNA sequence, a terminator sequence, and a second AAV ITR sequence.

In some embodiments, an AAV vector can comprise a first AAV ITR sequence, a first promoter sequence, a first snRNA sequence, a termination sequence, a second promoter sequence, second snRNA sequence, a second termination sequence and a second AAV ITR sequence. In some embodiments, an AAV vector can comprise a first AAV ITR sequence, a first promoter sequence, a first snRNA sequence, a termination sequence, a second promoter sequence, a second snRNA sequence, a second termination sequence, a third promoter sequence, a third snRNA sequence, a third termination sequence, and a second AAV ITR sequence. In some embodiments, an AAV vector can comprise a first AAV ITR sequence, a first promoter sequence, a first snRNA sequence, a termination, a second promoter sequence, second snRNA sequence, a second termination sequence and a second AAV ITR sequence. In some embodiments, an AAV vector can comprise a first AAV ITR sequence, a first promoter sequence, a first snRNA sequence, a termination, a second promoter sequence, a second snRNA sequence, a second termination sequence, a third promoter sequence, a third snRNA sequence, a third termination sequence, a fourth promoter sequence, a fourth snRNA sequence, a fourth termination sequence, and a second AAV ITR sequence.

In some embodiments of the compositions and methods of the disclosure, the viral vector comprises a sequence isolated or derived from an adeno-associated virus (AAV). In some embodiments, the viral vector comprises an ITR sequence or a capsid sequence that is isolated or derived from an AAV of serotype AAV1, AAV2, AAV3, AAV4, AAV5, AAV6, AAV7, AAV8, AAV9, AAV10, AAVrh10, AAV11 or AAV12. In some embodiments, the AAV serotype is AAVrh.74. In one embodiment, the AAV vector comprises a modified capsid. In one embodiment the AAV vector is an AAV2-Tyr mutant vector. In one embodiment the AAV vector comprises a capsid with a non-tyrosine amino acid at a position that corresponds to a surface-exposed tyrosine residue in position Tyr252, Tyr272, Tyr275, Tyr281, Tyr508, Tyr612, Tyr704, Tyr720, Tyr730 or Tyr673 of wild-type AAV2. See also WO 2008/124724 incorporated herein in its entirety. In some embodiments, the AAV vector comprises an engineered capsid. AAV vectors comprising engineered capsids include without limitation, AAV2.7m8, AAV9.7m8, AAV2 2tYF, and AAV8 Y733F). In some embodiments, the capsid is a ubiquitination resistant capsid. In another embodiment, the ubiquitination capsid is an AAV2 capsid comprising tyrosine (Y) and serine (S) mutations. In another embodiment, the AAV2 capsid comprises Y, S and threonine (T) mutations. In another embodiment, the AAV2 capsid includes, without limitation, AAV2 capsid mutants such as T455V, T491V, T550V, T659V, Y444+500+730F, and Y444+500+730F+T491V. In some embodiments, the viral vector is replication incompetent. In some embodiments, the viral vector is isolated or recombinant (rAAV). In some embodiments, the viral vector is self-complementary (scAAV). In some embodiments, the viral vector is single-stranded (ssAAV).

In some embodiments, the snRNAs provided herein are comprised within a single-stranded AAV (ssAAV). In some embodiments, the snRNAs provided herein are comprised within a self-complementary AAV (scAAV). The single-stranded nature of the parvoviral genome requires the use of cellular mechanisms to provide a complementary-strand for gene expression. This cellular recruitment activity is considered a rate-limiting factor in the efficiency of transduction and gene expression in parvoviruses and parvoviral particles. The use of an scAAV versus an ssAAV remedies this well known issue by packaging both strands as a single duplex DNA molecule (or inverted repeat genome) that can fold into dsDNA as a result of a self-complementary viral genome sequence. In this regard, the requirement for DNA synthesis or base-pairing between multiple viral genomes is eliminated.

### AAV ITR Sequences

In some embodiments of the compositions and methods of the disclosure, an AAV inverted terminal repeat sequence can comprise any AAV ITR sequence known in the art. In some embodiments, an AAV ITR sequence can comprise or consist of an AAV1 ITR sequence, an AAV2 ITR sequence, an AAV3 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAVrh10 ITR sequence, an AAV11 ITR sequence, an AAV12 ITR sequence, an AAV13 ITR sequence, or an AAVrh74 ITR sequence.

In some embodiments the ITR sequence can comprise a modified AAV ITR sequence.

In some embodiments, an AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 89 or SEQ ID NO: 90.

In some embodiments, an AAV vector provided herein comprises a first and a second AAV ITR sequence. In some embodiments, a first AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 89 or SEQ ID NO: 90 and a second AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 89 or SEQ ID NO: 90. In some embodiments the first AAV ITR sequence is positioned at the 5′ of an AAV vector. In some embodiments the second AAV ITR sequence is positioned at the 3′ of an AAV vector.

In some embodiments, a first AAV ITR sequence comprises the sequence set forth in SEQ ID NO: 89 or SEQ ID NO: 90. In some embodiments, a second AAV ITR sequence comprises the sequence set forth in SEQ ID NO: 89 or SEQ ID NO: 90. In some embodiments, an AAV vector provided herein comprises a first AAV ITR sequence comprising the sequence set forth in SEQ ID NO: 89 and a second AAV ITR sequence comprising the sequence set forth in SEQ ID NO: 90. In some embodiments the first AAV ITR sequence is positioned at the 5′ of an AAV vector. In some embodiments the second AAV ITR sequence is positioned at the 3′ of an AAV vector.

In some embodiments of the compositions and methods of the disclosure, the viral vector comprises a sequence isolated or derived from an adeno-associated virus (AAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

### Non-Viral Vectors

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

### Promoter Sequences

Gene therapy and RNA-targeting snRNA gene therapy compositions of the disclosure comprise promoter sequences derived from an snRNA.

A “promoter” is a control sequence that is a region of a polynucleotide sequence at which initiation and rate of transcription are controlled. It may contain genetic elements at which regulatory proteins and molecules may bind such as RNA polymerase and other transcription factors.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. An “enhancer” is a region of DNA that can be bound by activating proteins to increase the likelihood or frequency of transcription.

In some embodiments of the compositions and methods of the disclosure, an expression vector, viral vector or non-viral vector provided herein, includes without limitation, vector elements such as a buffer sequence derived human genomic sequences downstream from an snRNA and as such will have the capability to encoding multiple snRNAs from a single construct.

In some embodiments, the snRNA constructs disclosed herein comprise bidirectional promoters to express snRNAs.

In another embodiment, the vector configurations can comprise linker(s), signal sequence(s), and/or tag(s).

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV2-Tyr mutant vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAVrh8 vector, an AAV9 vector, an AAV.rh10 vector, a modified AAV.rh10 vector, an AAVrh.74, an AAV.rh32/33 vector, a modified AAV.rh32/33 vector, an AAV.rh43 vector, a modified AAV.rh43 vector, an AAV.rh64R1 vector, and a modified AAV.rh64R1 vector, an AAV-Tyr mutant vector, AAV-Tyr-Ser mutant vector, AAV-Tyr-Ser-Thr mutant vector and any combinations or equivalents thereof

### Lentiviral Vectors

In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### Nucleic Acids

An NOI (nucleotide sequence of interest) includes, without limitation, any nucleotide sequence or transgene capable of being delivered by a vector. NOIs can be synthetic, derived from naturally occurring DNA or RNA, codon optimized, recombinant RNA/DNA, cDNA, partial genomic DNA, and/or combinations thereof. The NOI can be a coding region or partial coding region, but need not be a coding region. An NOI can be RNA/DNA in a sense or anti-sense orientation. An NOI can be an snRNA. NOIs are also referred herein, without limitation, as transgenes, heterologous sequences, genes, therapeutic genes. An NOI may also encode an RNA (ribonucleoprotein complex) a POI (protein of interest), a partial POI, a mutated version or variant of a POI. A POI may be analogous to or correspond to a wild-type protein. A POI may also be a fusion protein or ribonucleoprotein complex such as an snRNP. In some embodiments RNA sequences disclosed herein may be represented as DNA sequences and it is within the ability of the skilled artisan to derive the sequence of an RNA sequence from a DNA sequence. For example, spacer sequences of the disclosure can represent uracil bases as either a U or T. The skilled artisan would readily understand that an RNA sequence can interchangeably use a T or U to indicate a uracil.

### Codon Optimization

In some embodiments, NOIs or transgenes such as nucleic acid sequences of the disclosure are codon optimized nucleic acid sequences.

In some embodiments. NOIs or transgenes or GOIs such as nucleic acid sequences encoding RNA-targeting snRNAs of the disclosure are codon optimized nucleic acid sequences. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased transcription or translation in a human subject relative to a wild-type or non-codon optimized nucleic acid sequence.

In some embodiments, a codon optimized nucleic acid sequence exhibits increased stability. In some embodiments, a codon optimized nucleic acid sequence exhibits increased stability through increased resistance to hydrolysis. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased stability relative to a wild-type or non-codon optimized nucleic acid sequence. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased resistance to hydrolysis in a human subject relative to a wild-type or non-codon optimized nucleic acid sequence.

In some embodiments, a codon optimized nucleic acid sequence can comprise no donor splice sites. In some embodiments, a codon optimized nucleic acid sequence can comprise no more than about one, or about two, or about three, or about four, or about five, or about six, or about seven, or about eight, or about nine, or about ten donor splice sites. In some embodiments, a codon optimized nucleic acid sequence comprises at least one, or at least two, or at least three, or at least four, or at least five, or at least six, or at least seven, or at least eight, or at least nine, or at least ten fewer donor splice sites as compared to a non-codon optimized nucleic acid sequence.

Without wishing to be bound by theory, the removal of donor splice sites in the codon optimized nucleic acid sequence can unexpectedly and unpredictably increase expression of protein of interest in vivo, as cryptic splicing is prevented. Moreover, cryptic splicing may vary between different subjects, meaning that the expression level of a protein comprising donor splice sites may unpredictably vary between different subjects. Such unpredictability is unacceptable in the context of human therapy. Accordingly, the codon optimized nucleic acid sequences which lacks donor splice sites, unexpectedly and surprisingly allows for increased expression of the protein in human subjects and regularizes expression of the protein across different human subjects.

In some embodiments, a codon optimized nucleic acid sequence can have a GC content that differs from the GC content of the non-codon optimized nucleic acid sequence encoding the RNA-targeting snRNA. In some embodiments, the GC content of a codon optimized nucleic acid sequence is more evenly distributed across the entire nucleic acid sequence, as compared to the non-codon optimized nucleic acid sequence.

Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some embodiments, a codon optimized nucleic acid sequence can have fewer repressive microRNA target binding sites as compared to the non-codon optimized nucleic acid sequence. In some embodiments, a codon optimized nucleic acid sequence can have at least one, or at least two, or at least three, or at least four, or at least five, or at least six, or at least seven, or at least eight, or at least nine, or at least ten, or at least ten fewer repressive microRNA target binding sites as compared to the non-codon optimized nucleic acid sequence.

Without wishing to be bound by theory, by having fewer repressive microRNA target binding sites, the codon optimized nucleic acid sequence unexpectedly exhibits increased expression in a human subject.

Provided herein are the nucleic acid sequences encoding the gene therapy compositions or RNA-targeting snRNA systems for use in gene transfer and expression techniques described herein. It should be understood, although not always explicitly stated that the sequences provided herein can be used to provide the expression product as well as substantially identical sequences that encode an RNA or express and produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

The NOIs or nucleic acid sequences (e.g., polynucleotide sequences) disclosed herein may be codon-optimized which is a technique well known in the art. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences coding for, e.g., an snRNA, can be generated. In some embodiments, such a sequence is optimized for expression in a host or target cell, such as a host cell used to express the snRNA or a cell in which the disclosed methods are practiced (such as in a mammalian cell, e.g., a human cell). Codon preferences and codon usage tables for a particular species can be used to engineer isolated nucleic acid molecules encoding an snRNA that takes advantage of the codon usage preferences of that particular species. For example, the snRNA disclosed herein can be designed to have codons that are preferentially used by a particular organism of interest. In one example, an snRNA nucleic acid sequence is optimized for expression in human cells, such as one having at least 70%, at least 80%, at least 85%, at least 90%, at least 92%, at least 95%, at least 98%, or at least 99% sequence identity to its corresponding wild-type or originating nucleic acid sequence. In some embodiments, an isolated nucleic acid molecule encoding at least one snR-NA (which can be part of a vector) includes at least one snRNA coding sequence that is codon optimized for expression in a eukaryotic cell, or at least one snRNA coding sequence codon optimized for expression in a human cell. In one embodiment, such a codon optimized snRNA coding sequence has at least 80%, at least 85%, at least 90%, at least 92%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes snRNA having at least 85%, at least 90%, at least 92%, at least 95%, at least 96%, at least 97%, at least 98%, at least 99%, or 100% sequence identity to its corresponding wild-type or originating sequence. In another embodiment, a variety of clones containing functionally equivalent nucleic acids may be routinely generated, such as nucleic acids which differ in sequence but which encode the same snRNA sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG. TTA, or TTG; serine can be encoded by TCT, TCC, TCA, TCG, AGT, or AGC: asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W. H. 5 Freeman and Co., NY).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10× SSC; formamide concentrations of about 0% to about 25%; and wash solutions from about 4×SSC to about 8×SSC. Examples of moderate hybridization conditions include: incubation temperatures of about 40° C. to about 50° C.: buffer concentrations of about 9×SSC to about 2×SSC: formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC: formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Cells

In some embodiments of the compositions and methods of the disclosure, a cell of the disclosure is a prokaryotic cell.

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell.

In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, a somatic cell of the disclosure is a neuronal cell. In one embodiment, a cell or cells of a patient treated with compositions disclosed herein include, without limitation, central nervous system (neurons), peripheral nervous system (neurons), peripheral motor neurons, and/or sensory neurons. In one embodiment, a neuronal cell is a glial cell.

In some embodiments of the compositions and methods of the disclosure, a somatic cell of the disclosure is a fibroblast or an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the disclosure, a somatic cell is an ocular cell. An ocular cell includes, without limitation, corneal epithelial cells, keratyocytes, retinal pigment epithelial (RPE) cells, lens epithelial cells, iris pigment epithelial cells, conjunctival fibroblasts, non-pigmented ciliary epithelial cells, trabecular meshwork cells, ocular choroid fibroblasts, conjunctival epithelial cells, In some embodiments, an ocular cell is a retinal cell or a corneal cell. In one embodiment, a retinal cell is a photoreceptor cell or a retinal pigment epithelial cell. In another embodiment, a retinal cell is a ganglion cell, an amacrine cell, a bipolar cell, a horizontal cell, a Müller glial cell, a rod cell, or a cone cell. In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### Methods of Use

The disclosure provides a method of encoding an RNA or expressing an NOI in a cell using the snRNA systems disclosed herein. In one embodiment, the disclosure provides a method of modifying an RNA or the activity of a protein encoded by an RNA molecule comprising contacting the composition of the disclosure and the target RNA molecule under conditions suitable for binding to the target RNA molecule.

The disclosure provides a method of modifying the level of expression of a target RNA molecule of the disclosure or a protein encoded by the target RNA molecule comprising contacting the composition of the disclosure and a cell comprising the target RNA molecule under conditions suitable for binding to the target RNA molecule. In some embodiments, the cell is in vivo, in vitro, ex vivo or in situ. In some embodiments, the composition of the disclosure comprises a vector comprising at least one snRNA sequence. In some embodiments, the vector is an AAV.

The disclosure provides a method of modifying the level of expression of an RNA molecule of the disclosure or a protein encoded by the RNA molecule comprising contacting the composition of the disclosure and the RNA molecule under conditions suitable for knocking down, blocking, splicing, multi-targeting, or editing the target RNA. In some embodiments, the vector is an AAV.

The disclosure provides a method of editing adenosine (A) to inosine (I) in an RNA molecule of interest comprising contacting the RNA molecule of interest with an snRNA molecule of the disclosure. In some embodiments, guanine to adenosine point mutations can be corrected using the snRNA molecules of the disclosure. In some embodiments, an A to I editing event can result in a single amino acid mutation in an expressed protein of interest. An A to I editing event can remove a stop codon in an mRNA sequence. In some embodiments, the stop codon is a premature stop codon. Thus, removal of said premature stop codon can lead to rescued translation of a protein of interest.

The disclosure provides a method of modifying a target RNA or an activity of a protein encoded by an RNA molecule comprising contacting the composition and a cell comprising the RNA molecule under conditions suitable knocking down, blocking, splicing, multi-targeting, or editing the target RNA. In some embodiments, the cell is in vivo, in vitro, ex vivo or in situ. In some embodiments, the composition comprises a vector comprising the snRNA sequences disclosed herein. In some embodiments, the vector is an AAV.

The disclosure provides a method of treating a disease or disorder comprising administering to a subject a therapeutically effective amount of an snRNA composition of the disclosure.

### MBNL1 Disorders

The pathogenic sequestration of MBNL1 (Muscleblind-like protein 1), caused by the CUG-repeat expansion of the DMPK 3′UTR, functionally reduces availability of MBNL1 protein and contributes to the phenotypic features seen in Myotonic Dystrophy Type 1. A proposed solution to alleviate the reduced activity of MBNL1 is through the enhancement of MBNL1 protein. One proposed strategy to boost expression of MBNL1 is through the disruption of post-transcriptional regulation of miRNA targeting whereby snRNA-mediated A to I editing of the critical miRNA seed region prevents the recruitment of MBNL1-targeting miRNAs. MBNL1-targeting mir30 and mir23 bind to adenosine nucleotides within their miRNA seed region and are thus good candidates for seed-base pairing disruption by A to I editing.

Accordingly, the disclosure provides a method of treating Myotonic Dystrophy Type 1 (DM1) in a patient in need of such treatment comprising administering to the patient a therapeutically effective amount of an snRNA composition of the disclosure. In some embodiments, the snRNA composition of the disclosure targets MBNL1.

### SOD1 Disorders

Amyotrophic lateral sclerosis (ALS) is a neurodegenerative disease characterized by muscle atrophy caused by the selective loss of motor neurons. A common molecular contributor to the development of ALS is the toxic gain of function activity of superoxide dismutase (SOD1) which acts in a dominant manner. Because of this, SOD1 has emerged as a leading target for patients with ALS, and a promising candidate for snRNA-mediated regulation. By targeting the adenosine within the start codon of SOD1, snRNA mediated A to I editing prevents the ribosome machinery from recognizing the initiation site, thereby reducing the expression of mutant SOD1 protein.

Accordingly, the disclosure provides a method of treating Amyotrophic lateral sclerosis (ALS) in a patient in need of such treatment comprising administering to the patient a therapeutically effective amount of an snRNA composition of the disclosure. In some embodiments, the snRNA composition of the disclosure targets SOD1.

Most MPS (Mucopolysaccharidosis) I-H patients have a premature stop codon mutation in one or both alleles and are unable to synthesize a full-length polypeptide causing a loss of enzyme activity. The two alpha-L-iduronidase (IDUA) gene premature stop codons, Q70X and W402X, are the most common (70%) mutations in MPS I patients (also referred to as Hurler syndrome), a rare, recessively inherited lysosomal storage disorder, a progressive, multi-systemic disease caused by a reduced or absent IDUA enzyme activity secondary to biallelic loss-of-function variants in the IDUA. snRNA-mediated targeting of IDUA W402X/Exon 9 provides improved stability and less immunogenicity/off-targets compared to prime editors or traditional gene therapy enzyme replacement.

Accordingly, the disclosure provides a method of treating Hurler syndrome in a patient in need of such treatment comprising administering to the patient a therapeutically effective amount of an snRNA composition of the disclosure. In some embodiments, the snRNA composition of the disclosure targets IDUA.

Huntington's disease (HD) is a neurodegenerative disorder caused by the expansion of the CAG trinucleotide-repeats found in the gene encoding huntingtin (HTT), encoding toxic polyglutamine expansions within exon 1. Targeting of the HTT mRNA to lower its expression has been proposed as a therapeutic strategy, one that can be achieved by disrupting the start codon of HTT by snRNA-mediated editing.

Accordingly, the disclosure provides a method of treating Huntington's Disease in a patient in need of such treatment comprising administering to the patient a therapeutically effective amount of an snRNA composition of the disclosure. In some embodiments, the snRNA composition of the disclosure targets HTT.

snRNA compositions of the disclosure can also be implemented in targeting amyloid-beta precursor protein (APP), which contributes to the formation of aggregate in the brain in Alzheimer's diseases. Editing of the APP gene can lead to a reduction in protein expression.

Accordingly, the disclosure provides a method of treating Alzheimer's Disease in a patient in need of such treatment comprising administering to the patient a therapeutically effective amount of an snRNA composition of the disclosure. In some embodiments, the snRNA composition of the disclosure targets APP.

Nonsense mutations observed in methyl CpG binding protein 2 (MeCP2) have been demonstrated as a major cause of Rett syndrome, a neurodevelopmental disorder seen primarily in females. U7 snRNA targeting MeCP2 mutations such as R168X, R255X, and 270X are good candidates for RNA editing.

Accordingly, the disclosure provides a method of treating Rett Syndrome in a patient in need of such treatment comprising administering to the patient a therapeutically effective amount of an snRNA composition of the disclosure. In some embodiments, the snRNA composition of the disclosure targets MeCP2.

The aberrantly expressed double homeobox 4 (DUX4) gene is linked to the development of facioscapulohumeral muscular dystrophy (FSHD). Although it is normally silenced in healthy individuals, patients with FSHD experience sporadic de-repression of DUX4. Targeting the start codon DUX4 transcript with snRNAs to repress its expression in FSHD patients could offer a therapeutic benefit to patients. In addition, this RNA editing strategy to repression expression can be extended towards editing the polyadenylation sequence of DUX4.

Accordingly, the disclosure provides a method of treating facioscapulohumeral muscular dystrophy (FSHD) in a patient in need of such treatment comprising administering to the patient a therapeutically effective amount of an snRNA composition of the disclosure. In some embodiments, the snRNA composition of the disclosure targets DUX4.

Individuals with mutations to SERPINA1 gene which encodes Alpha-1 antitrypsin, develop alpha-1 antitrypsin deficiency, a disorder that predisposes patients to lung and liver disease. Mutations such as SERPINA1 E342K can be targeted for snRNA-mediated editing.

Accordingly, the disclosure provides a method of treating alpha-1 antitrypsin deficiency in a patient in need of such treatment comprising administering to the patient a therapeutically effective amount of an snRNA composition of the disclosure. In some embodiments, the snRNA composition of the disclosure targets SERPINA1.

The disclosure provides a method of treating a disease in a patient in need of such treatment comprising administering to the patient a therapeutically effective amount of an snRNA composition of the disclosure, wherein the composition comprises a vector comprising snRNA sequences disclosed herein, wherein the composition modifies, reduces, destroys, knocks down or ablates a level of expression of a target RNA (compared to the level of expression of a target RNA treated with a non-targeting (NT) control or compared to no treatment). In another embodiment, the level of reduction is 1-fold or greater. In another embodiment, the level of reduction is 2-fold, 3-fold, 4-fold, 5-fold, 6-fold, 7-fold, 8-fold, 9-fold or 10-fold. In another embodiment, the level of reduction is 10-fold or greater. In another embodiment, the level of reduction is between 10-fold and 20-fold. In another embodiment, the level of reduction is 11-fold, 12-fold, 13-fold, 14-fold, 15-fold, 16-fold, 17-fold, 18-fold, 19-fold, or 20-fold. In another embodiment, the gene therapy compositions disclosed herein when administered to a patient lead to 20%-100% destruction of the target RNA. In one embodiment, the % elimination of the toxic RNA is any of 20-99%, 25%-99%, 50%-99%, 80%-99%, 90%-99%. 95%-99%. In one embodiment, the % elimination is 90%, 91%, 92%, 93%, 94%, 95%, 96%, 97%, 98%, or 99%. In another embodiment, % elimination is complete elimination or 100% elimination of the target RNA.

The disclosure provides a method of treating a disease in a patient in need of such treatment comprising administering to the patient a therapeutically effective amount of an snRNA composition of the disclosure, wherein the composition comprises a vector comprising snRNA sequences disclosed herein, wherein the composition enhances expression of a protein encoded by a target RNA of interest (compared to the level of expression of a protein encoded by a target RNA treated with a non-targeting (NT) control or compared to no treatment). In another embodiment, the level of enhancement is 1-fold or greater. In another embodiment, the level of enhancement is 2-fold, 3-fold, 4-fold, 5-fold, 6-fold, 7-fold, 8-fold, 9-fold or 10-fold. In another embodiment, the level of enhancement is 10-fold or greater. In another embodiment, the level of enhancement is between 10-fold and 20-fold. In another embodiment, the level of enhancement is 11-fold, 12-fold, 13-fold, 14-fold, 15-fold, 16-fold, 17-fold, 18-fold, 19-fold, or 20-fold. In another embodiment, the gene therapy compositions disclosed herein when administered to a patient lead to 0.0001%-100% increase of the protein encoded by a target RNA. In one embodiment, the % increase of the protein encoded by a target RNA is any of 20-99%, 25%-99%, 50%-99%, 80%-99%, 90%-99%, 95%-99%. In one embodiment, the % increase is 90%, 91%, 92%, 93%, 94%, 95%, 96%, 97%, 98%, or 99%.

In some embodiments of the methods of the disclosure, a subject of the disclosure has been diagnosed with a disease to be treated. In some embodiments, the subject of the disclosure presents at least one sign or symptom of a disorder or disease to be treated. In some embodiments, the subject of the disclosure presents at least one sign or symptom of a disease.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure, a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure, a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure, a subject of the disclosure is a mammal. In some embodiments, a subject of the disclosure is a non-human mammal.

In some embodiments of the methods of the disclosure, a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure, a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject via intracerebral administration. In some embodiments, the composition of the disclosure is administered to the subject by an intrastriatal route. In some embodiments, the composition of the disclosure is administered to the subject by a stereotaxic injection or an infusion. In some embodiments, the composition is administered to the brain. In some embodiments of the methods of the disclosure, a composition of the disclosure is administered to the subject locally.

In some embodiments, the compositions disclosed herein are formulated as pharmaceutical compositions. Briefly, pharmaceutical compositions for use as disclosed herein may comprise a protein(s) or a polynucleotide encoding the protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like: carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for routes of administration, such as e.g., oral, enteral, topical, transdermal, intranasal, and/or inhalation; and for routes of administration via injection or infusion such as, e.g., intravenous, intramuscular, subpial, intrathecal, intraparenchymal, intrathecal, intrastriatal, subcutaneous, intradermal, intraperitoneal, intratumoral, intravenous, intraocular, and/or parenteral administration. In certain embodiments, the compositions of the present disclosure are formulated for intracerebral or intrastriatal administration.

### Examples

### Example 1: SnRNA Targeting Stop Codons in mCherry/GFP Reporter Assay

### Materials and Methods

Day 1: seed HeLa cells at 100 k/ml in 96 well plate (clear F-bottom, black edges) with 0.1 ml per well
Day 2: transfect with lipofectamine
Dilute all DNA to 100 ng/uL.
Make master mix A (1 μL P3000+25 uL OMEM+0.25 uL P03685 per sample=(22×) 550 uL OMEM+22 uL P3000+5.5 uL P03685 reporter plasmid)
Gentle vortex.
Add 25 μL per sample of master mix A to 5 uL diluted DNA samples.
Gentle vortex.
Make master mix of 25 μL per sample OMEM+1.25 μl lipofectamine per sample (22×=550 uL OMEM+27.5 uL lipofectamine).
Gentle vortex, and pipette 25 μL per sample to diluted DNA with P3000.
Gently mix, incubate for 10 mins at RT, and add 10 uL drop-wise to cells in quadruplicate.
Day 4: read fluorescence and extract RNA
Remove media and add 100 uL PBS to each well.
Read fluorescence for GFP and mCherry on BioTek reader.
Take images on microscope.
Extract RNA using 96-well Qiagen RNeasy Plus kit. Amplify using forward primer 5′-CGCCTACAACGTCAACATCA-3′(SEQ ID NO: 93) and reverse primer 5′-TGGTGCAGATGAACTTCAGG-3′(SEQ ID NO: 94) using standard Promega GoTaq protocol with 59C annealing, 40 s extension, 32× cycles. Send PCR products for Sanger sequencing with PCR primers.

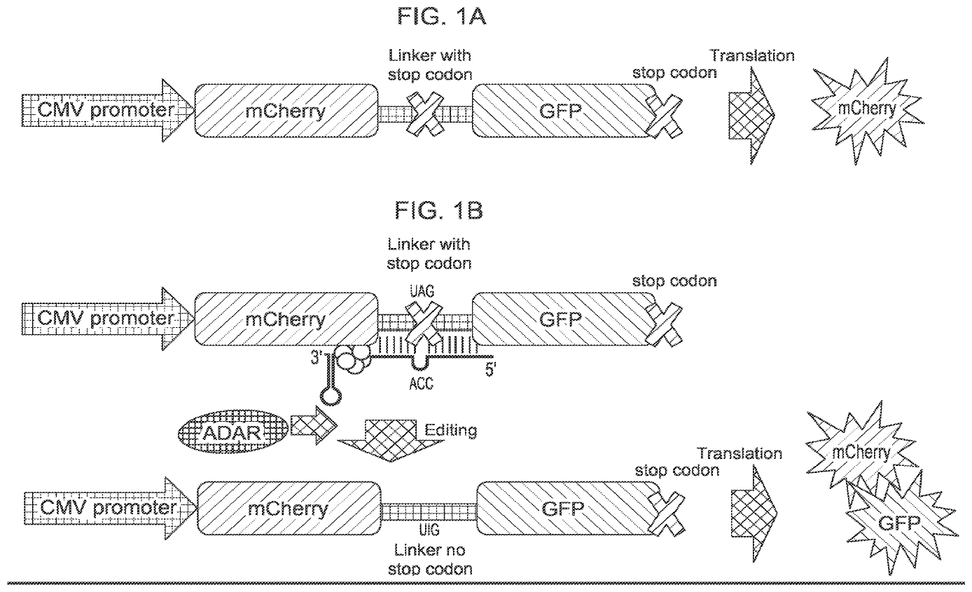

Purpose. To determine whether snRNPs comprising a target binding sequence with a single basepair mismatch targeting the stop codon in the reporter vector depicted in FIG. 1A are capable of inducing an A to I editing event to restore expression of GFP.

### Rationale:

U7 snRNA can be programmed to target mRNAs by replacing its histone mRNA annealing sequence with a sequence complementary to a target of interest, in this case a stop codon, to create an snRNA (snRNA). The stop codon-targeting constructs were tested to determine whether they can induce an A to I editing event mediated by ADAR proteins. Such an editing event removes the stop codon allowing dual expression of both mCherry and GFP (FIG. 1B).

Stop-codon targeting snRNA constructs and controls, including a non-targeting snRNA, were transiently transfected into HeLa cells expressing the mCherry/GFP reporter construct. The mCherry/GFP reporter construct is depicted in Table 1.

TABLE 1
mCherry/GFP reporter construct - P03414 pcDNA3.1_CMV-mCherry-
linker_stop_GFP for editing

| CMV | GACATTGATTATTGACTAGTTATTAATAGTAATCAATTACGGGGTCATTAGTTCATAGCC |
|---|---|
| enhancer/prom | CATATATGGAGTTCCGCGTTACATAACTTACGGTAAATGGCCCGCCTGGCTGACCGCCCA |
| oter | ACGACCCCCGCCCATTGACGTCAATAATGACGTATGTTCCCATAGTAACGCCAATAGGGA |
| | CTTTCCATTGACGTCAATGGGTGGAGTATTTACGGTAAACTGCCCACTTGGCAGTACATC |
| | AAGTGTATCATATGCCAAGTACGCCCCCTATTGACGTCAATGACGGTAAATGGCCCGCCT |
| | GGCATTATGCCCAGTACATGACCTTATGGGACTTTCCTACTTGGCAGTACATCTACGTAT |
| | TAGTCATCGCTATTACCATGGTGATGCGGTTTTGGCAGTACATCAATGGGCGTGGATAGC |
| | GGTTTGACTCACGGGGATTTCCAAGTCTCCACCCCATTGACGTCAATGGGAGTTTGTTTT |
| | GGCACCAAAATCAACGGGACTTTCCAAAATGTCGTAACAACTCCGCCCCATTGACGCAAA |
| | TGGGCGGTAGGCGTGTACGGTGGGAGGTCTATATAAGCAGAGCT (SEQ ID NO: 95) |
| 5′ UTR | CTCTGGCTAACTAGAGAACCCACTGCTTACTGGCTTATCGAAATTAATACGACTCACTAT |
| | AGGGAGACCCAAGCTGGCTAGCGTTTAAACGGGCCCT (SEQ ID NO: 96) |
| mCherry | ATGGTGAGCAAGGGCGAGGAGGATAACATGGCCATCATCAAGGAGTTCATGCGCTTCAAG |
| | GTGCACATGGAGGGCTCCGTGAACGGCCACGAGTTCGAGATCGAGGGCGAGGGCGAGGGC |
| | CGCCCCTACGAGGGCACCCAGACCGCCAAGCTGAAGGTGACCAAGGGTGGCCCCCTGCCC |
| | TTCGCCTGGGACATCCTGTCCCCTCAGTTCATGTACGGCTCCAAGGCCTACGTGAAGCAC |
| | CCCGCCGACATCCCCGACTACTTGAAGCTGTCCTTCCCCGAGGGCTTCAAGTGGGAGCGC |
| | GTGATGAACTTCGAGGACGGCGGCGTGGTGACCGTGACCCAGGACTCCTCCCTGCAGGAC |
| | GGCGAGTTCATCTACAAGGTGAAGCTGCGCGGCACCAACTTCCCCTCCGACGGCCCCGTA |
| | ATGCAGAAGAAGACCATGGGCTGGGAGGCCTCCTCCGAGCGGATGTACCCCGAGGACGGC |
| | GCCCTGAAGGGCGAGATCAAGCAGAGGCTGAAGCTGAAGGACGGCGGCCACTACGACGCT |
| | GAGGTCAAGACCACCTACAAGGCCAAGAAGCCCGTGCAGCTGCCCGGCGCCTACAACGTC |
| | AACATCAAGTTGGACATCACCTCCCACAACGAGGACTACACCATCGTGGAACAGTACGAA |
| | CGCGCCGAGGGCCGCCACTCCACCGGCGGCATGGACGAGCTGTACAAG (SEQ ID NO: |
| | 97) |
| Linker and | CTGCAGGGCGGAGGAGGCAGCGGCGGAGGAGGCAGCGGCGGAGGAGGCAGCGCCTGCTCG |
| target stop | CGATGCTAGAGGGCTCTGCCA (SEQ ID NO: 98) |
| codon | |
| (bolded) | |
| GFP | GTGAGCAAGGGCGAGGAGCTGTTCACCGGGGTGGTGCCCATCCTGGTCGAGCTGGACGGC |
| | GACGTAAACGGCCACAAGTTCAGCGTGTCCGGCGAGGGCGAGGGCGATGCCACCTACGGC |
| | AAGCTGACCCTGAAGTTCATCTGCACCACCGGCAAGCTGCCCGTGCCCTGGCCCACCCTC |
| | GTGACCACCCTGACCTACGGCGTGCAGTGCTTCAGCCGCTACCCCGACCACATGAAGCAG |
| | CACGACTTCTTCAAGTCCGCCATGCCCGAAGGCTACGTCCAGGAGCGCACCATCTTCTTC |
| | AAGGACGACGGCAACTACAAGACCCGCGCCGAGGTGAAGTTCGAGGGCGACACCCTGGTG |
| | AACCGCATCGAGCTGAAGGGCATCGACTTCAAGGAGGACGGCAACATCCTGGGGCACAAG |
| | CTGGAGTACAACTACAACAGCCACAACGTCTATATCATGGCCGACAAGCAGAAGAACGGC |
| | ATCAAGGTGAACTTCAAGATCCGCCACAACATCGAGGACGGCAGCGTGCAGCTCGCCGAC |
| | CACTACCAGCAGAACACCCCCATCGGCGACGGCCCCGTGCTGCTGCCCGACAACCACTAC |
| | CTGAGCACCCAGTCCGCCCTGAGCAAAGACCCCAACGAGAAGCGCGATCACATGGTCCTG |
| | CTGGAGTTCGTGACCGCCGCCGGGATCACTCTCGGCATGGACGAGCTGTACAAGTAA |
| | (SEQ ID NO: 99) |
| 3′ UTR | AATTCCACCACACTGGACTAGTGGATCCGAGCTCGGTACCAAGCTTAAGTTTAAACCGCT |
| | GATCAGCCTCGA (SEQ ID NO: 100) |
| BGH polyA | CTGTGCCTTCTAGTTGCCAGCCATCTGTTGTTTGCCCCTCCCCCGTGCCTTCCTTGACCC |
| | TGGAAGGTGCCACTCCCACTGTCCTTTCCTAATAAAATGAGGAAATTGCATCGCATTGTC |
| | TGAGTAGGTGTCATTCTATTCTGGGGGGTGGGGTGGGGCAGGACAGCAAGGGGGAGGATT |
| | GGGAAGACAATAGCAGGCATGCTGGGGATGCGGTGGGCTCTATGG (SEQ ID NO: |
| | 101) |

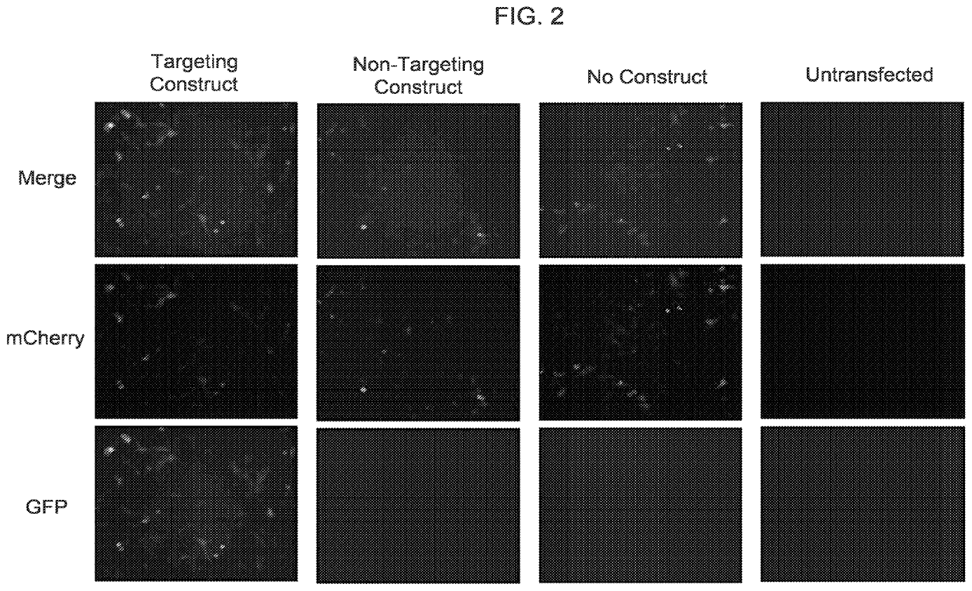

Cells were seeded and fixed onto chamber slides, and imaged using a fluorescent microscope to detect mCherry and GFP expression (FIG. 2). The stop-codon targeting snRNA induced GFP expression in addition to mCherry whereas the non-targeting snRNA, dummy (no construct) transfection, and untransfected cells displayed mCherry expression only (FIG. 2).

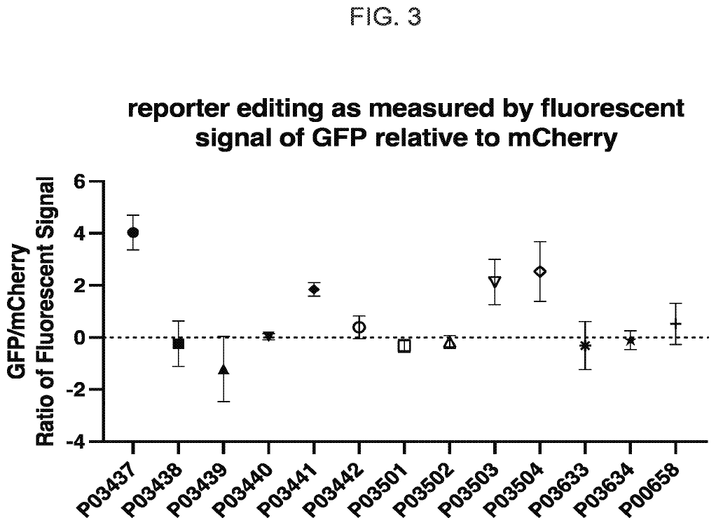

Next, further stop-codon targeting snRNA constructs were evaluated. Evaluated snRNA molecules include those detailed in Table 2. Constructs were developed that comprised varying combinations of additional features outside of the stop-codon targeting sequence including engineered stem loops, sm binding domains with or without an ADAR recruiting domain and/or a 5′ interaction stabilization domain (5′ISD). Non-targeting versions of each construct were also evaluated. The ratio of GFP to mCherry expression was determined for each construct and depicted in FIG. 3. Non-targeting snRNA did not induce GFP expression. Multiple stop-codon targeting snRNA induced GFP expression indicating an A to I editing event occurred enabling read through of the construct enabling mCherry and GFP expression. Importantly, it was observed that A to I editing was achieved by constructs comprising an ADAR recruiting domain and constructs lacking an ADAR recruiting domain.

TABLE 2
stop-codon targeting snRNA constructs

| ID | Name | Description |
|---|---|---|
| P03437 | pcDNA3.1_U7_U7-5′ISD- | 5′ISD; stop codon targeting sequence (70 nt); Sm |
| | mCherry-Stop 70 | binding domain (SmOPT); engineered stem loop |
| | | (eSL) |
| P03438 | pcDNA3.1_U7_U7-5′ISD- | 5′ISD; stop codon targeting sequence (151 nt); |
| | mCherry-Stop 151 | Sm binding domain (SmOPT); engineered stem |
| | | loop (eSL) |
| P03439 | pcDNA3.1_U7_U7-5′ISD- | 5′ISD; non-targeting sequence (70 nt); Sm |
| | nt 70 | binding domain (SmOPT); engineered stem loop |
| | | (eSL) |
| P03440 | pcDNA3.1_U7_U7-5′ISD- | 5′ISD; non-targeting sequence (151 nt); Sm |
| | nt 151 | binding domain (SmOPT); engineered stem loop |
| | | (eSL) |
| P03441 | pcDNA3.1_U7_U7-ISD- | 5′ISD; ADAR recruiting domain; stop codon |
| | ADAR-recruiting-mCherry- | targeting sequence (70 nt); Sm binding domain |
| | Stop 70 | (SmOPT); engineered stem loop (eSL) |
| P03442 | pcDNA3.1_U7_U7-ISD- | 5′ISD; ADAR recruiting domain; stop codon |
| | ADAR recruiting-mCherry- | targeting sequence (151 nt); Sm binding domain |
| | Stop 151 | (SmOPT); engineered stem loop (eSL) |
| P03501 | pcDNA3.1_U7_U7-ISD | 5′ISD; ADAR recruiting domain; non-targeting |
| | ADAR recruiting-nt 70 | sequence (70 nt); Sm binding domain (SmOPT); |
| | | engineered stem loop (eSL) |
| P03502 | pcDNA3.1_U7_U7-ISD | 5′ISD; ADAR recruiting domain; non-targeting |
| | ADAR recruiting-nt 151 | sequence (151 nt); Sm binding domain (SmOPT); |
| | | engineered stem loop (eSL) |
| P03503 | pcDNA3.1_U7_U7-ADAR- | ADAR recruiting domain; stop codon targeting |
| | recruiting-mCherry-Stop 70 | sequence (70 nt); Sm binding domain (SmOPT); |
| | | engineered stem loop (eSL) |
| P03504 | pcDNA3.1_U7_U7-ADAR- | ADAR recruiting domain; stop codon targeting |
| | recruiting-mCherry-Stop | sequence (151 nt); Sm binding domain (SmOPT); |
| | 151 | engineered stem loop (eSL) |
| P03633 | pcDNA3.1_U7_U7-ADAR | ADAR recruiting domain; non-targeting sequence |
| | recruiting-nt 70 | (70 nt); Sm binding domain (SmOPT); engineered |
| | | stem loop (eSL) |
| P03634 | pcDNA3.1_U7_U7-ADAR | ADAR recruiting domain; non-targeting sequence |
| | recruiting-nt 151 | (151 nt); Sm binding domain (SmOPT); |
| | | engineered stem loop (eSL) |
| P00658 | pcDNA3.1 | Mammalian expression vector with CMV |
| | | promoter |

### Example 2: SnRNA-Mediated SOD1 RNA Editing in 293T Cells

### Materials and Methods

Transfection of 293T cells: 293T cells were plated onto 24-well TC-treated plates at 5E4 cells per well and allowed to grow overnight. The following day, cells were transfected using 500 ng of plasmid DNA per reaction along with 1 uL P3000 and 1.5 uL Lipofectamine 3000 reagent. Cells were allowed to grow for 48-72 hours until they were harvested for downstream or RNA analysis.

RNA preparation and analysis by qRT-PCR: Total RNA was prepared with 500 uL Trizol (Invitrogen) and extracted using the Zymo direct-zol RNA microprep kit (Zymo Research). 50 ng of RNA was reverse transcribed using the qScript Ultra SuperMix (Qantabio) and followed by PCR. Samples were PCR purified (Qiagen) and sent for Sanger sequencing.

Protein extraction and analysis by Western blot: Samples used for protein extraction were lysed using 50 uL RIPA buffer supplemented with cOmplete™, mini protease inhibitor cocktail (Roche). Samples were incubated for 30 minutes on ice, vortexing every 5 minutes. Samples were then spun at 14,000×g for 10 minutes. The supernatant was then transferred to a new tube and protein concentrations were quantified using Pierce™ BCA Protein Assay. 1.5 ug of sample was loaded on the Jess (Protein Simple).

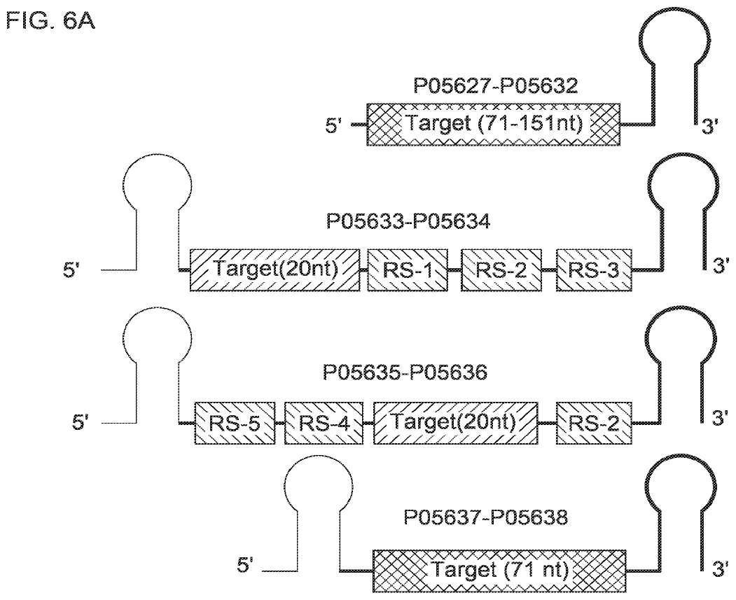

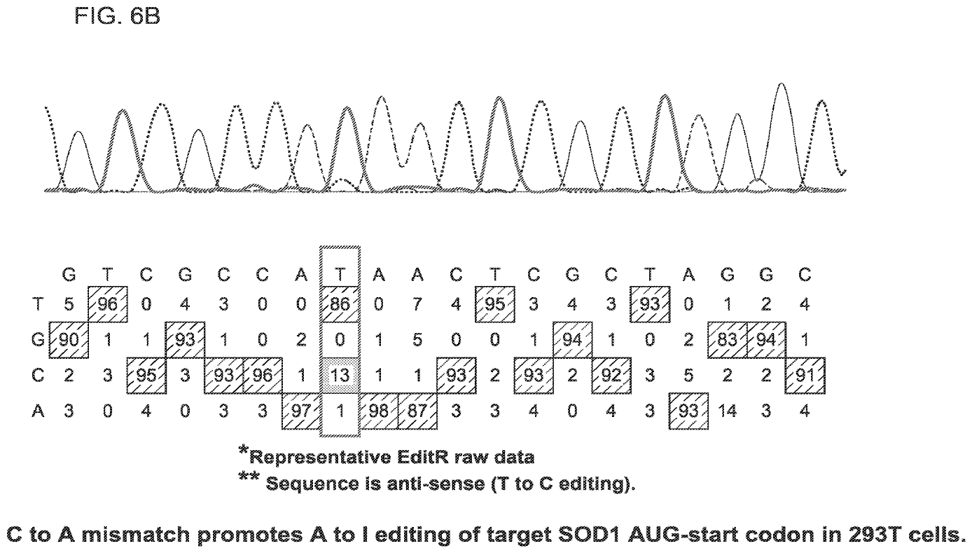

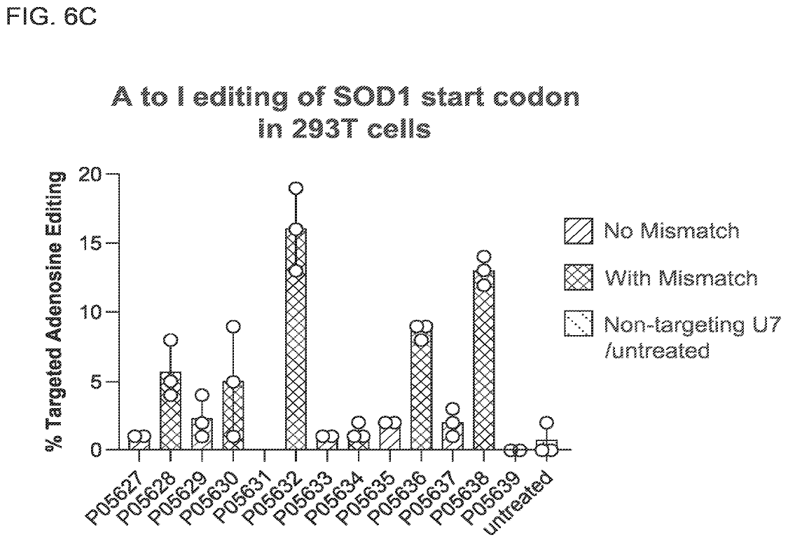

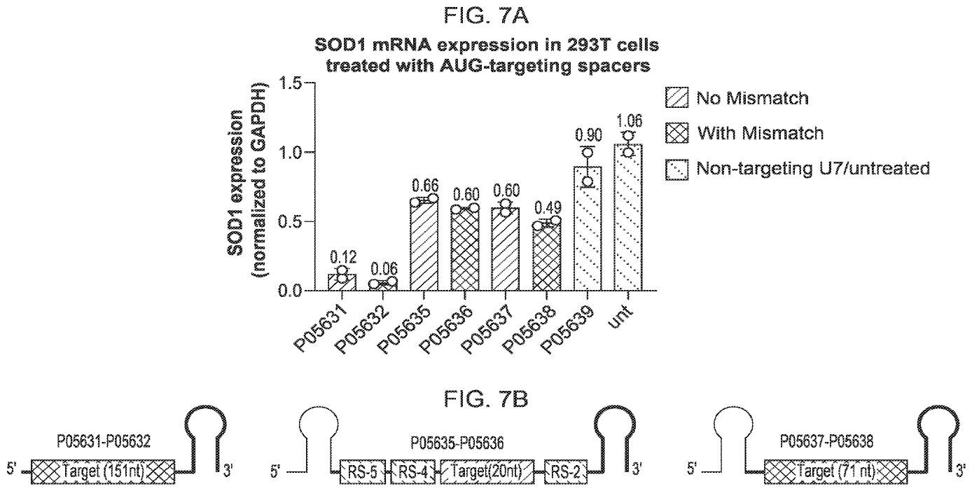

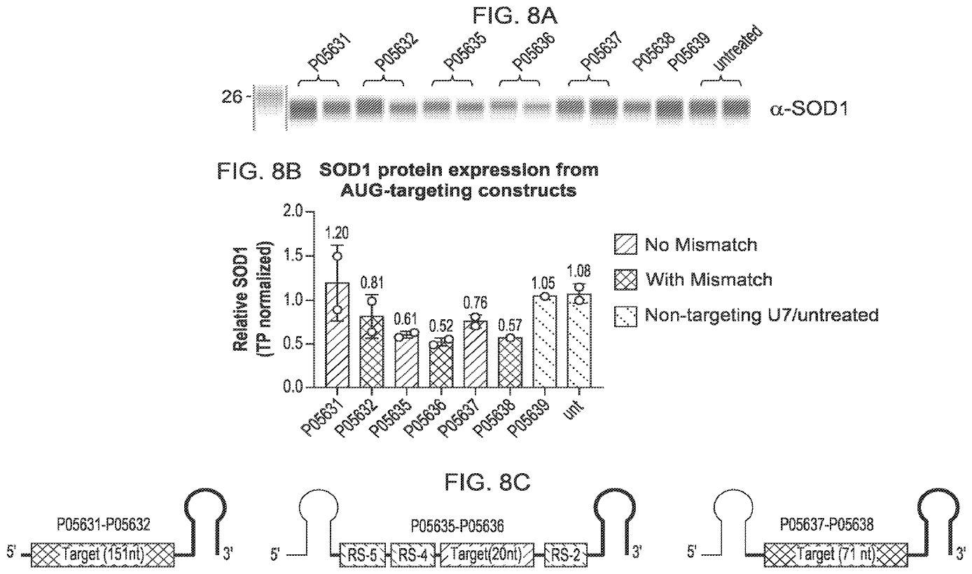

Conclusion: Transient transfections of 293T cells with constructs targeting the SOD1 codon promoted RNA editing. Editing was enhanced by the presence of a Cytosine to Adenosine mismatch. Targeting the SOD1 start codon led to a decrease in SOD1 expression. See FIGS. 6-8. This strategy may be utilized to target other therapeutically relevant transcripts such as the aberrant expression of the double homeobox 4 (DUX4) for treating skeletal muscle deterioration and weakness in Facioscapulohumeral muscular dystrophy (FSHD).

### Example 3: ScAAV snRNA-Mediated Recruitment of ADAR for mRNA Editing in Hurler Patient Derived Skeletal Muscle Cells

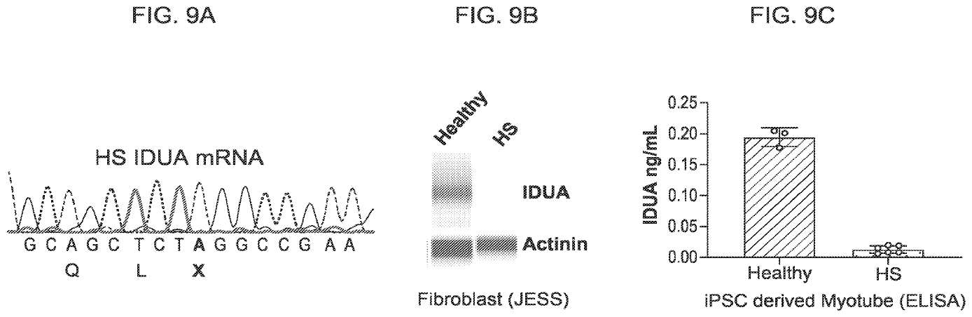

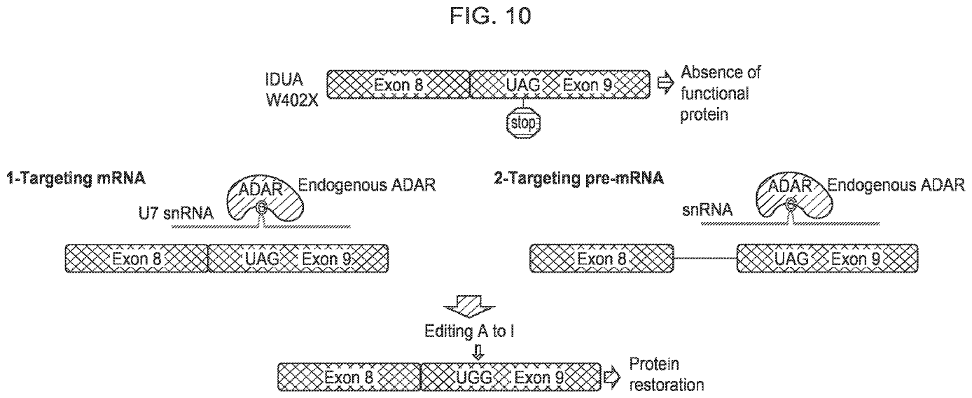

Most MPS I-H patients have a premature stop codon mutation in one or both alleles and are unable to synthesize a full-length polypeptide causing a loss of enzyme activity. The two alpha-L-iduronidase (IDUA) gene premature stop codons. Q70X and W402×, are the most common (70%) mutations in MPS I patients. snRNA-mediated targeting of IDUA W402×/Exon 9 provides improved stability and less immunogenicity/off-targets compared to prime editors or traditional gene therapy enzyme replacement. See FIGS. 9-10.

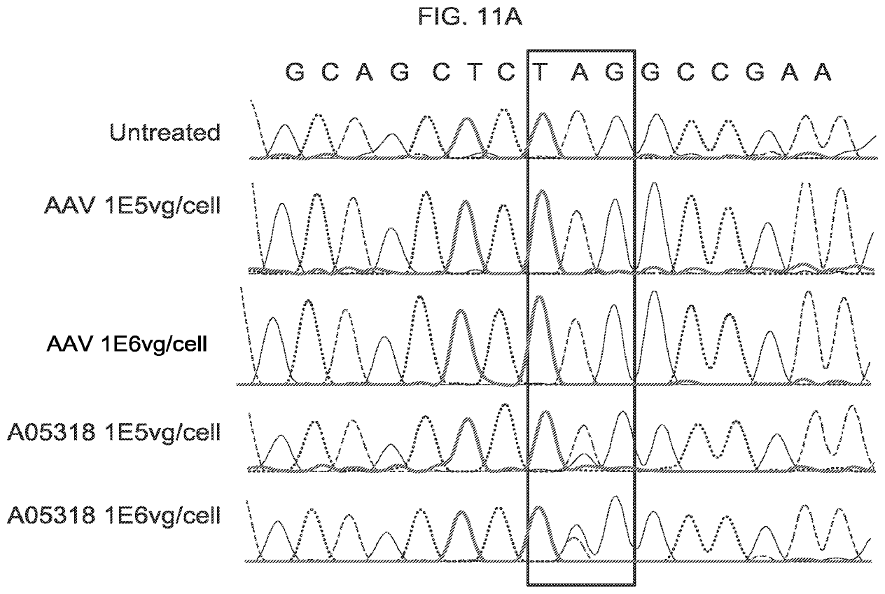

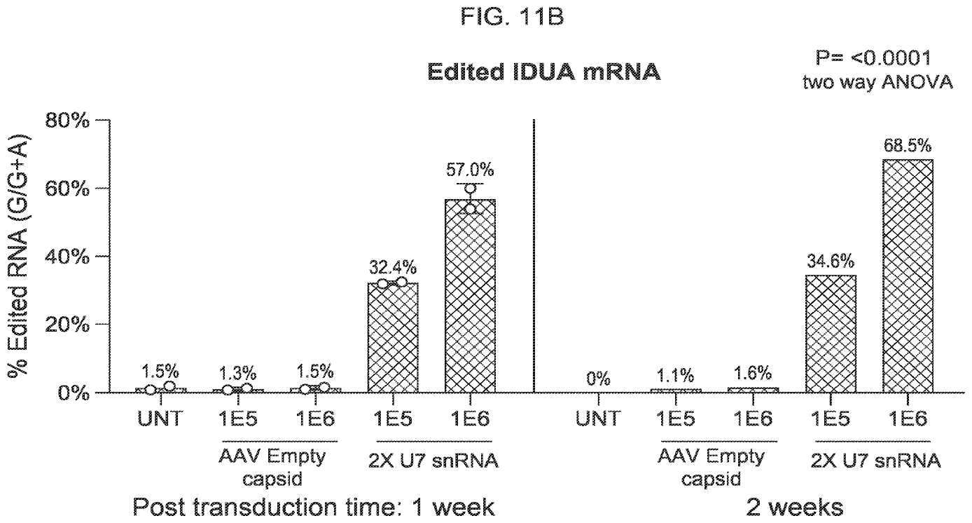

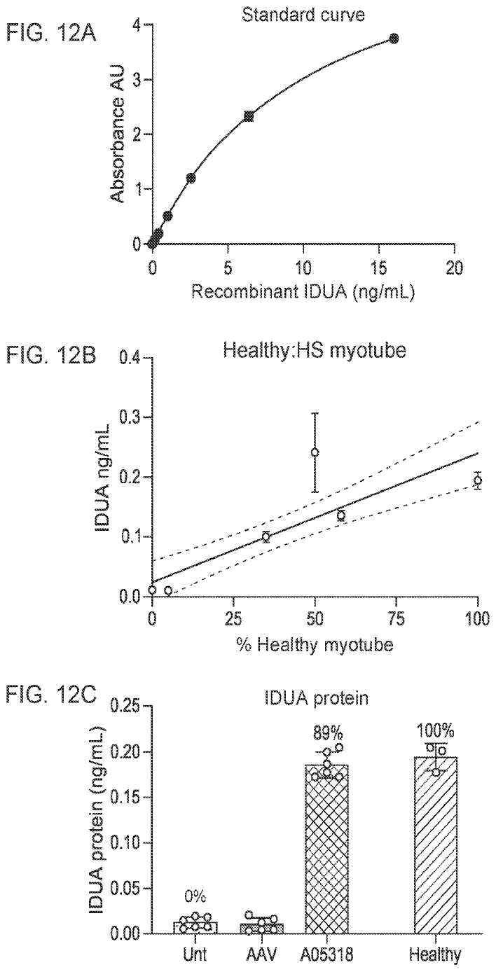

Hurler Syndrome myotubes were transduced with scAAV A05318 targeting W420X Exon 9. Sanger sequencing was carried out for IDUA exon 9 depicting A>I (A>G) editing in the A05318 treated myotube at 1E5 and 1E6 vg/cell after 7 days. RT-PCR of IDUA editing efficiencies was performed and chromatograms were analyzed by EditR. Untreated cells and AAV empty capsids at MOI 1E5 and 1E6 vg/cell serve as negative controls for the assay. Untreated and AAV empty capsid treated cells serve as negative controls to show lack of edited RNA in this cell line. This study shows dose-dependent snRNA-mediated editing of IDUA in the patient myotubes. See FIG. 11. ELISA (Thermo Fisher EH247RB) was performed and results indicate snRNA treatment restores IDUA enzyme in the Hurler myotubes, an increasing level of IDUA protein level in the supernatant of co-cultures of healthy and untreated Hurler Syndrome patient derived myotubes at 100:0, 35:65, 50:50, 58:42, and 0:100 ratios. IDUA protein levels in the supernatant of A05318 treated myotubes at 1E6 vg/cell after 10 days. Untreated cells and AAV empty capsids at 1E6 vg/cell serve as negative controls for the assay. Percent healthy was calculated keeping healthy myotubes as 100%. Untreated and AAV empty capsids treated cells serve as negative controls to show lack of edited RNA in this cell line. See FIG. 12.

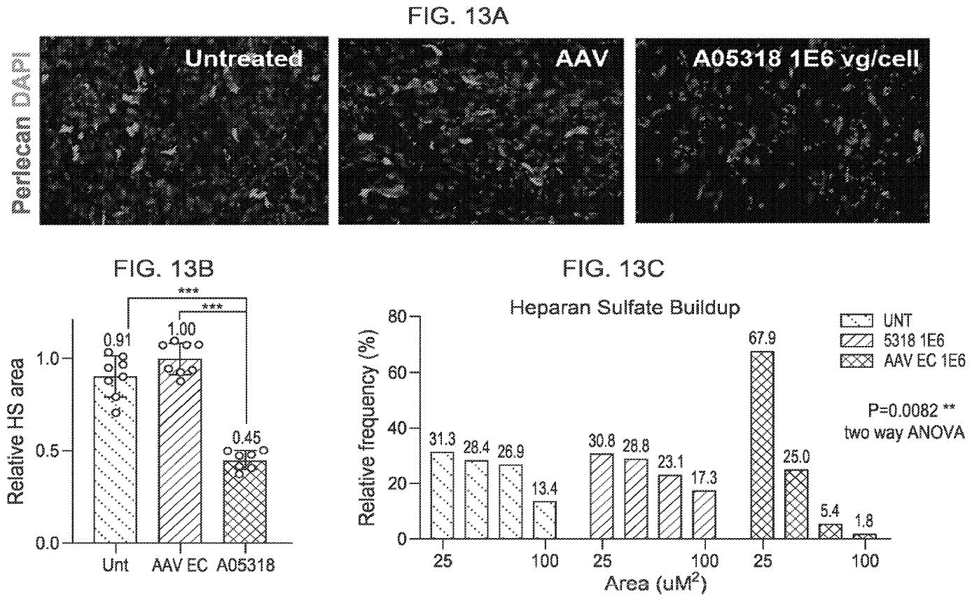

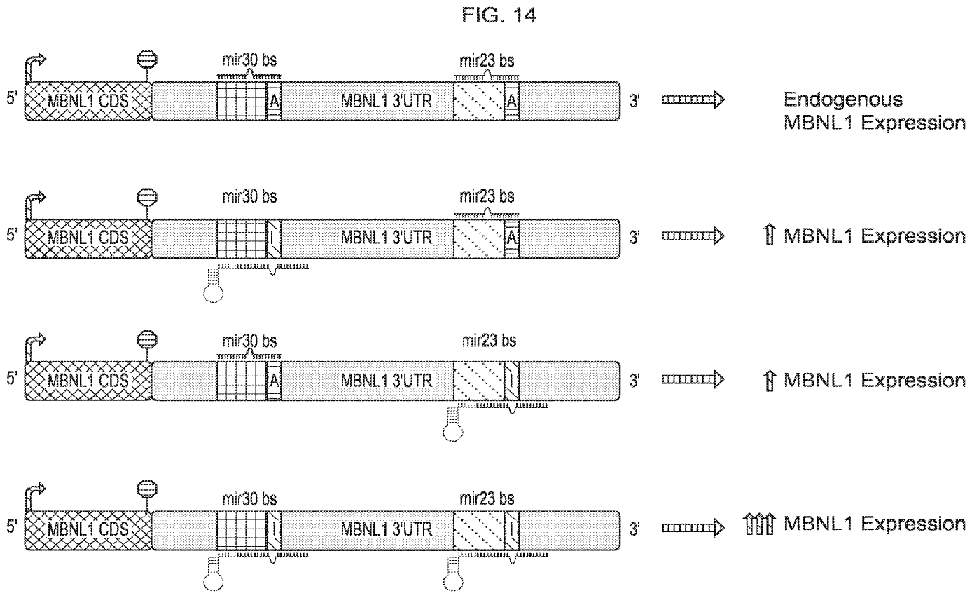

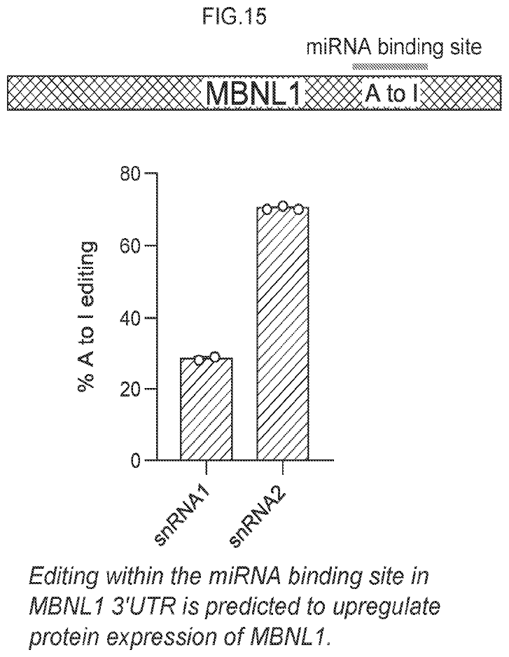

Myotubes were treated with A05318 and analyzed with immunofluorescence. Images showed reduced Perlecan staining 10 days after treatment of myotubes with A05318 at 1E6 vg/cell with Perlecan antibody (Abcam, red). Untreated cells and AAV empty capsids treated cells at 1E6 vg/cell serve as negative controls to show high baseline staining in this cell line. DAPI stain (blue) was used to detect nuclei. See FIG. 13.

Conclusions: This study shows efficient snRNA-mediated editing in Hurler patient cells. High,sustained levels of RNA editing at 7 and 14 days post transduction was observed. A05318 showed dose-dependent editing at the 2 doses 1E5 and 1E6 vg/cells tested. scAAV9 U7-snRNA A05318 treatment restores functional IDUA protein and reduced GAG (Perlecan) buildups at 10 days post transduction.

### Example 4: Targeting and Editing MBNL1 Binding Sites Increases MBNL Expression

The pathogenic sequestration of MBNL1 (Muscleblind-like protein 1), caused by the CUG-repeat expansion of the DMPK 3′UTR, functionally reduces availability of MBNL1 protein and contributes to the phenotypic features seen in Myotonic Dystrophy Type 1 (DM1). A proposed solution to alleviate the reduced activity of MBNL1 is through the enhancement of MBNL1 protein. One proposed strategy to boost expression of MBNL1 is through the disruption of post-transcriptional regulation of miRNA targeting whereby snRNA-mediated A to I editing of the critical miRNA seed region prevents the recruitment of MBNL1-targeting miRNAs. MBNL1-targeting mir30 and mir23 bind to adenosine nucleotides within their miRNA seed region and are thus good candidates for seed-base pairing disruption by A to I editing.

Adenosine to Inosine Editing of the MBNL 3′UTR at the miR30 Seed Binding Site

MNBL1 targeting snRNA, snRNA 1 and snRNA 2 containing antisense sequences to MBNL1 3′UTR at miR30 binding site and with a mismatch to the A of the seed sequence were transfected into HEK cells for 72 hours. RNA was extracted, reverse transcription was performed and cDNA was amplified with PCR. The resulting DNA amplicon was subjected to sanger sequencing. Chromatographs of amplicon were analyzed by EditR

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or embodied herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### Other Embodiments

While particular embodiments of the disclosure have been illustrated and described, various other changes and modifications can be made without departing from the spirit and scope of the disclosure. The scope of the appended claims includes all such changes and modifications that are within the scope of this disclosure.

What is claimed is:

1. An RNA-targeting nucleic acid molecule comprising a small nuclear RNA (snRNA) molecule, wherein the snRNA molecule comprises at least one targeting sequence, wherein the targeting sequence has at least one base-pairing mismatch, an Sm binding domain (SmBD), and an snRNA stem loop.

2. The RNA-targeting nucleic acid molecule of claim 1, wherein the snRNA further comprises an Adenosine Deaminase That Acts on RNA (ADAR) recruiting domain.

3. The RNA-targeting nucleic acid molecule of claim 1, wherein the snRNA further a 5′ interaction stability domain (5′ISD).

4. The RNA-targeting nucleic acid molecule of claim 1, wherein the snRNA stem loop is a native stem loop or an engineered stem loop (eSL).

5. The RNA-targeting nucleic acid molecule of claim 1, wherein the snRNA is a U1 snRNA, U2 snRNA, U3 snRNA, U4 snRNA, U5 snRNA, U6 snRNA, or U7 snRNA.

6. The RNA-targeting nucleic acid molecule of claim 5, wherein the snRNA is a U7 snRNA.

7. The RNA-targeting nucleic acid molecule of claim 1, wherein the targeting sequence binds an mRNA or pre-mRNA sequence.

8. The RNA-targeting nucleic acid molecule of claim 1, wherein the targeting sequence binds a start codon, a stop codon, or a splicing regulatory sequence.

9. The RNA-targeting nucleic acid molecule of claim 8, wherein the splicing regulatory sequence is a branch point adenosine (bpA) sequence, 3′ acceptor splice site (3′ ss) sequence, or exonic splicing enhancer (ESE) sequence.

10. The RNA-targeting nucleic acid molecule of claim 1, wherein the base-pairing mismatch is an adenosine (A)-cytosine (C) mismatch.

11. The RNA-targeting nucleic acid molecule of claim 10, wherein the adenosine is comprised within the mRNA or pre-mRNA sequence.

12. The RNA-targeting nucleic acid molecule of claim 10, wherein the cytosine is comprised within the snRNA targeting sequence.

13. The RNA-targeting nucleic acid molecule of claim 1, wherein the targeting sequence is at least 50 nucleotides in length.

14. The RNA-targeting nucleic acid molecule of claim 1, wherein the snRNA further comprises one or more additional targeting sequences.

15. The RNA-targeting nucleic acid molecule of claim 14, wherein the one or more additional targeting sequences have at least one base-pairing mismatch.

16. The RNA-targeting nucleic acid molecule of claim 1, wherein the Sm binding domain (SmBD) is selected from the group consisting of a U1, U2, U4, and U5 SmBD.

17. The RNA-targeting nucleic acid molecule of claim 3, wherein the interaction stability domain (5′ISD) comprises ggagt, cctct, ggaggt, cctcct, agccag, ggaag, gaagaag, gttg, ccgaa, taaggag, gaag, or ggctt.

18. The RNA-targeting nucleic acid molecule of claim 2, wherein the ADAR recruiting domain comprises a nucleotide sequence set forth in SEQ ID NO: 88.

19. The RNA-targeting nucleic acid molecule of claim 4, wherein the snRNA stem loop comprises a nucleotide sequence set forth in any one of SEQ ID NOs: 1-41.

20. The RNA-targeting nucleic acid molecule of claim 1, wherein the targeting sequence binds a SOD1 RNA sequence.

21. The RNA-targeting nucleic acid molecule of claim 20, wherein the SOD1 RNA targeting sequence comprises a nucleotide sequence set forth in any one of SEQ ID NOs: 124-150.

22. The RNA-targeting nucleic acid molecule of claim 1, wherein the targeting sequence binds an IDUA RNA sequence.

23. The RNA-targeting nucleic acid molecule of claim 22, wherein the IDUA RNA targeting sequence comprises a nucleotide sequence set forth in any one of SEQ ID NOs: 153 or 154.

24. The RNA-targeting nucleic acid molecule of claim 1, wherein the targeting sequence binds an MBNL1 RNA sequence.

25. The RNA-targeting nucleic acid molecule of claim 24, wherein the MBNL1 RNA targeting sequence comprises a nucleotide sequence set forth in any one of SEQ ID NOs: 156-173.

26. The RNA-targeting nucleic acid molecule of claim 1, wherein the snRNA is operably linked to a U7 promoter or a U1 promoter.

27. The RNA-targeting nucleic acid molecule of claim 1, wherein the snRNA is operably linked to a U7 promoter and a U1 promoter.

28. The RNA-targeting nucleic acid molecule of claim 1, wherein the snRNA is operably linked to an snRNA downstream terminator (DT).

29. A vector comprising the snRNA of claim 1.

30. The vector of claim 29, wherein the vector is a viral vector or a non-viral vector.

31. The vector of claim 29, wherein the viral vector is an AAV vector.

32. The vector of claim 29, wherein the vector comprises multiple copies of the snRNA of claim 1.

33. The vector of claim 32, wherein the vector comprises 2, 3 or 4 copies of the snRNA.

34. The vector of claim 32, wherein each copy of the multiple copies of the snRNA is separated by a buffer sequence, wherein the buffer sequence is selected from the group consisting of SEQ ID NOS: 42-48.

35. A method of targeting one or more target RNAs of interest and blocking, knocking down, editing, or splicing the one or more target RNAs, comprising contacting the snRNA of claim 1 with a cell comprising the one or more target RNAs.

36. A method of editing adenosine (A) to inosine (I) in an RNA molecule of interest comprising contacting the RNA molecule of interest with an snRNA of claim 1.

37. The method of claim 36, wherein the A to I edit is performed by an ADAR molecule recruited by the snRNA.

38. A method of treating a disease or disorder in a subject in need thereof comprising administering to the subject an RNA-targeting nucleic acid molecule of claim 1 or an AAV vector of claim 31.

39. The method of claim 38, wherein the disease or disorder is Myotonic Dystrophy Type 1, Amyotrophic lateral sclerosis (ALS), or Hurler syndrome.

40. An RNA-targeting nucleic acid molecule comprising an snRNA system (snRNA), wherein the snRNA system comprises at least one targeting sequence, and an Sm binding domain (SmBD), and an snRNA stem loop, wherein the binding sequence has extensive complementarity to a target RNA.
